(19) United States
(12) Patent Application Publication
(10) Pub. No.: US 2012/0015368 A1
(43) Pub. Date: Jan. 19, 2012

(54) BIOMARKERS FOR EARLY DIAGNOSIS OF SYSTEMIC TISSUE FIBROSIS

(75) Inventors: Francesco Del Galdo, Leeds (GB); Sergio A. Jimenez, Philadelphia, PA (US)

(73) Assignee: THOMAS JEFFERSON UNIVERSITY, Philadelphia, PA (US)

(21) Appl. No.: 13/184,697

(22) Filed: Jul. 18, 2011

Related U.S. Application Data
(60) Provisional application No. 61/364,852, filed on Jul. 16, 2010.

(51) Int. Cl. C12Q 1/68; C12M 1/34; G01N 30/96; G01N 33/566; G01N 33/573

(52) U.S. Cl. 435/6.12; 436/501; 435/7.4; 422/69; 435/287.2; 422/430

(57) ABSTRACT

Embodiments of the invention provides methods, devices and kits for determining the likelihood of an individual having an active fibrotic condition and/or early diagnosis, and subsequently prognosis evaluation of an individual having an active fibrotic condition such as systemic sclerosis (SSc) and/or nephrogenic systemic fibrosis (NSF) by measuring the levels of several biomarkers: α-enolase (ENO1), reticulocalbin 3 (RCN-3), alpha smooth muscle actin (α-SMA), reticulocalbin 1 (RCN-1) and pigment epithelium-derived factor (PEDF) in the individual.

### CROSS REFERENCE TO RELATED APPLICATION

This application claims benefit under 35 U.S.C. §119(e) of the U.S. Provisional Application No. 61/364,852 filed Jul. 16, 2010, the contents of which are incorporated herein by reference in its entirety.

### GOVERNMENT SUPPORT

This invention was made with Government support under grant No. R01AR19616 awarded by the National Institute of Health. The government has certain rights in the invention.

### BACKGROUND OF INVENTION

Fibrotic disorders, which include systemic sclerosis (SSc), idiopathic pulmonary fibrosis (IPF), cirrhosis of the liver, and the newly recognized nephrogenic systemic fibrosis (NSF), are characterized by abnormal and excessive deposition of collagen and other extracellular matrix (ECM) components in various tissues. Although their etiology is quite diverse, the presence of ECM-producing fibroblasts displaying an activated phenotype in the affected tissues is typical of all fibrotic diseases. Fibroblast activation is characterized by a marked increase in the transcriptional activity of the genes encoding type I and type III collagens and fibronectin, initiation of the expression of alpha-smooth muscle actin (α-SMA), and the reduction of ECM degradative activities. Activated fibroblasts display contractile properties resulting from the expression of stress fibers containing α-SMA, and their pro-fibrotic activation is part of a complex set of molecular and biochemical changes that are conserved for multiple sequential passages in vitro.

The most frequent systemic fibrotic disorder is SSc which is a rare chronic disease of unknown cause. It is a clinically heterogeneous, systemic disorder which affects the connective tissue of the skin, internal organs and the walls of blood vessels. It is characterized by alterations of the microvasculature, disturbances of the immune system and by massive deposition of collagen and other matrix substances in the connective tissue. The most apparent symptoms are diffuse fibrosis, and vascular abnormalities in the skin, joints, and internal organs (especially the esophagus, lower GI tract, lung, heart, and kidney). Other common symptoms include Raynaud's phenomenon (RP), polyarthralgia, dysphagia, heartburn, and swelling and eventually skin tightening and joint contractures of the fingers, hands and wrists. Basic functions of various cell types (endothelial cells, T-lymphocytes, monocytes, fibroblasts, mast cells) as well as the production and effects of cytokines, growth factors, and adhesion molecules are known to be involved in the development of this disease. The term scleroderma was introduced for SSc in 1874 because the skin was the most obvious organ involved. The extensive involvement of internal organs has only been realized in the second half of the 20th century.

When the skin is affected, the skin develops more compact collagen fibers in the reticular dermis, epidermal thinning, loss of rete pegs, and atrophy of dermal appendages. T lymphocytes may accumulate, and extensive fibrosis in the dermal and subcutaneous layers develops. In the nail folds, capillary loops dilate and some microvascular loops are lost. In the extremities, chronic inflammation and fibrosis of the synovial membrane and surfaces and peri-articular soft tissues occur.

When the internal organs and blood vessels are extensively involved, excess deposition of extracellular matrix can lead to hypomotility, reflux peptic esophagitis, Barrett's metaplasia and fibrotic structures in the esophagus; peristaltic abnormalities in the stomach and small and large intestine leading to pseudo-obstruction or malabsorption due to bacterial overgrowth; diverticular ulcerations, stenosis, chronic obstipation, megacolon and rectal prolapse in the large intestine; and pulmonary hypertension as a result of lung fibrosis and alveolitis; and kidney failure.

In general, the survival rate lies between 34 and 73%. It is, however, shorter with a poorer prognosis in men and older patients than in women and younger patients. SSc is about 4 times more common among women than men. It is most common in the 3rd to 5th decades of life and is rare in children. SSc can develop as part of mixed connective tissue disease. The overall 5 and 10 year survival rates are 86% and 69% respectively. Most patients die of cardiopulmonary or renal diseases. There is also an increase in cancer mortality, particularly of the lung.

The spectrum of sclerodermatous diseases comprises a wide variety of clinical entities such as morphea (patchy, linear, and generalized), pseudo-scleroderma and the overlap-syndromes with similar cutaneous and histopathologic manifestations. In addition, the complex pathophysiology of SSc, involving genetic factors, environmental factors, vascular and immune system functions, as well as fibroblasts and matrix substances, and the complexity of the internal organ involvement, results in sclerodermatous diseases often being studied as autoimmune or connective tissue diseases. Therefore, SSc has been a challenge for clinicians with regards to diagnostic procedures and therapeutic regimens. Clinical diagnosis of SSc often involves attention from several disciplines (e.g. dermatologists, rheumatologists, pulmonologists, nephrologists, and gastroenterologists) and may include invasive procedure such as a biopsy of the fibrotic tissue and/or skin for confirmation.

Nephrogenic systemic fibrosis (NSF), also known as nephrogenic fibrosing dermopathy (NFD), is a condition that occurs only in people with kidney insufficiency. It is a systemic disorder characterized by widespread tissue fibrosis, of which the most prominent and visible effects are in the skin. The widespread tissue fibrosis in the skin of NSF patients is similar to that seen in SSc patients. Fibrosis is the accumulation of excessive connective tissue. Patients with NSF describe swelling and tightening of the skin, usually limited to the extremities, but sometimes involving the trunk. The patient can suffer from excessive tension and hardness of the skin with slightly raised plaques, papules, or confluent papules; with or without pigmentary alteration and/or with biopsies showing increased numbers of fibroblasts, alteration of the normal pattern of collagen bundles seen in the dermis, and often increased dermal deposits of mucin. Skin histology shows increased numbers of fibroblast-like cells and haphazardly arranged collagen bundles in the dermis, extending into the fascia along subcutaneous septa. The condition may develop over a period of days to several weeks. In many cases, the skin thickening inhibits the flexion and extension of joints, resulting in contractures. Severely affected patients may be unable to walk, or fully extend the joints of their arms, hands, legs, and feet. Complaints of muscle weakness are common. Approximately 5% of patients have a rapidly progressive (fulminant) course. In these cases, patients can have widespread fibrosis involving the diaphragm, psoas muscles, proximal esophagus and intimal areas of vessels of the kidney and lungs. Complications resulting from these widespread fibroses leads to mortality.

As NSF is a rare and relatively recent diagnosis whose first description was in 2000, the natural history of the disease is not well understood. Clinical diagnosis of NSF involves several disciplines (e.g. rheumatologists, pulmonologists, nephrologists, and gastroenterologists etc.) and can include invasive procedure such as a biopsy of the fibrotic tissue and/or skin histology for confirmation.

Fibrotic disorders are complex and they are difficult to diagnose. Since fibrosis is a key contributor, and a multi-disciplinary and symptom-based approach is necessary to arrive at a clinical diagnosis, there is a need for simple diagnostic methods for fibrotic disorders, in particular, for early diagnosis so that appropriate and timely treatment can be implemented to reduce disorder progression and mortality.

### SUMMARY OF THE INVENTION

The inventors have discovered that fibroblast cells obtained from scleroderma tissues and/or fibroic tissues associated with systemic sclerosis (SSc) and/or nephrogenic systemic fibrosis (NSF) secrete an abundance of certain proteins into the extracellular environment. These proteins are α-enolase (ENO1), reticulocalbin 3 (RCN-3), alpha smooth muscle actin (α-SMA), reticulocalbin 1 (RCN-1), tropomyosin 4 (TMP4), alpha actin 1 (ACTA1), calreticulin (CALR) and pigment epithelium-derived factor (PEDF) (see Table 4). Under normal healthy conditions, these proteins are not found in great abundance extracellularly. Therefore, an increase in the amount of select proteins in the extracellular space can be used as biomarkers for early diagnosis of fibrotic disorders such as SSc and NSF, and prognosis evaluation of fibrotic disorders during a treatment regime or for monitoring of recurrence of the fibrosis conditions.

Embodiments of the invention provides a method for determining the likelihood of an individual having an active fibrotic condition and/or early diagnosis of an individual having an active fibrotic condition comprising: (a) measuring an amount of at least one biomarker protein selected from the group consisting of ENO1, RCN-3, and PEDF, wherein the at least one biomarker protein is selected alone or in combination with biomarker proteins RCN-1 and/or α-SMA, wherein the biomarker proteins are from a biological sample obtained from the individual; and (b) comparing the amount of step (a) with a reference amount, wherein the amount of step (a) is greater than the reference amount indicates that the individual has an increased likelihood of having an active fibrotic condition. In this embodiment, when the levels of biomarker proteins determined are two or more standard deviations greater than that of the reference amount, it is indicative that the individual has an increased likelihood of having an active fibrotic condition and should therefore be further evaluated by a clinician. Such individual can be one at risk of developing a fibrotic condition, e.g. having family history or had prior fibrotic condition that is under remission.

Embodiments of the invention also provides a method of prognostic evaluation in an individual diagnosed with and is being treated for an active fibrotic condition such as SSc, NSF or other chronic fibrotic diseases, the method comprising: (a) at a first time point, measuring an amount of at least one of biomarker protein selected from the group consisting of ENO1, RCN-3, α-SMA, RCN-1 and PEDF, wherein the measured biomarker protein is not RCN-1 only and not α-SMA only, wherein the biomarker proteins are from a biological sample obtained from the individual; (b) at a second time point, measuring an amount of at least one selected from the group consisting of ENO1, RCN-3, α-SMA, RCN-1 and PEDF, wherein the measured biomarker protein is not RCN-1 only and not α-SMA only, wherein the biomarker proteins are from a biological sample obtained from the individual, wherein the second time point is after the first time point, and wherein a combination of biomarker proteins ENO1, RCN-3, α-SMA, PEDF, and RCN-1 measured is the same at both the first and second time points; (c) comparing the amount of step (a) with the amount of step (b) for each respective biomarker protein, wherein the amount of step (b) is less than the amount of step (a) indicates that the treatment is effective in the individual having the active fibrotic condition. In this embodiment, when the levels of biomarker proteins in the second time point is lower than the levels of biomarker proteins measured in the first time point by at least two or more standard deviations lower, it is indicative that the treatment is effective in the individual having the active fibrotic condition.

In one embodiment, the invention also provides a method of prognostic evaluation in an individual diagnosed with and is being treated for an active fibrotic condition such as SSc, NSF or other chronic fibrotic diseases, the method comprising: (a) at a first time point, measuring an amount of at least one of biomarker protein selected from the group consisting of ENO1, RCN-3, α-SMA, RCN-1 and PEDF, wherein the measured biomarker protein is not RCN-1 only and not α-SMA only, wherein the biomarker proteins are from a biological sample obtained from the individual; (b) at a second time point, measuring an amount of at least one selected from the group consisting of ENO1, RCN-3, α-SMA, RCN-1 and PEDF, wherein the measured biomarker protein is not RCN-1 only and not α-SMA only, wherein the biomarker proteins are from a biological sample obtained from the individual; wherein the second time point is after the first time point, and wherein a combination of biomarker proteins ENO1, RCN-3, α-SMA, PEDF, and RCN-1 measured is the same at both first and second time points; (c) comparing the amount of step (a) with the amount of step (b) for each respective biomarker protein, wherein the amount of step (b) is greater than the amount of step (a) indicates that the treatment is not effective in the individual having the active fibrotic condition. In this embodiment, when the levels of biomarker proteins in the second time point of step (b) is greater than the levels of biomarker proteins measured in the first time point of step (a) by at least two or more standard deviations greater, it is indicative that the treatment is not effective in the individual having the active fibrotic condition.

In some embodiment, the methods described herein can be used to monitor any recurrence of any active fibrotic condition. In one embodiment, the method of prognosis evaluation is performed after the completion or termination of treatment with a therapy for an active fibrotic condition described herein, e.g. SSc or NSF, when the patient had been successfully been treated for the condition, is symptom free and has normal levels of the biomarker proteins described herein. In this embodiment, when the levels of biomarker proteins in the second time point is greater than the levels of biomarker proteins determined in the first time point by at least two or more standard deviations greater, it is indicative that the patient has a likelihood of recurrence of an active fibrotic condition described herein. In this embodiment, the first time point is when the individual is in remission and is symptom free, and the second time point is after the first time point and the individual is not having treatment for any fibrotic condition.

In some embodiments, it is contemplated that the methods described further comprise selecting an individual with an active fibrotic condition such as SSc, NSF or other chronic fibrotic diseases, or selecting an individual that is suspected of or likelihood of having an active fibrotic condition such as SSc, NSF or other chronic fibrotic diseases based on the known symptoms of an active fibrotic condition such as SSc, NSF or other chronic fibrotic diseases.

As used herein, the term “active fibrotic condition” refers to any condition in which fibrotic tissue, scar tissue, connective tissue, and/or extracellular matrix (ECM) material accumulates in one or more organs within the body in response to tissue injury (e.g., infection, autoimmune reaction, mechanical injury etc.) or for unknown etiology and that accumulation of tissues and ECM in tissues is currently in progress. As used herein, the expression “fibrosis conditions” and the expression “fibrotic conditions” are intended to have the same meaning. Specific examples include but are not limited to scleroderma, nephrogenic systemic fibrosis, fasciitis, scleromyxedema, retroperitoneal fibrosis, pulmonary fibrosis, liver fibrosis, chronic graft versus host disease and chronic allograft rejection.

In some embodiments, the biomarker proteins selected for measurement are those disclosed in Table 4 and/or Table 5. In some embodiments, combinations of more than one biomarker are measured for the methods described herein.

In some embodiments, the amount of any of the biomarker proteins ENO1, RCN-3, α-SMA, PEDF or RCN-1, including the biomarkers disclosed in Tables 4 and 5, measured is at least two standard deviations greater than the average reference amount. In some embodiments, the amount is between 2-10 standard deviations greater than the average reference including all possible whole integer numbers and including fractions of an integer between 2 and 10.

In some embodiments, the amount of ENO1, RCN-3, α-SMA, PEDF, or RCN-1 obtained in the second time point is at least two standard deviations lower or greater than those of the first time point. In other embodiments, the amount of ENO1, RCN-3, α-SMA, PEDF, or RCN-1 obtained in the second time point is between 2-10 standard deviations lower or greater than those of the first time point, including all possible whole integer numbers and including fractions of an integer between 2 and 10.

In one embodiment, the combinations of ENO1, RCN-3, α-SMA, PEDF, and RCN-1 selected for measurement are selected from the group consisting of ENO1 only, RCN-3 only, PEDF only, and all other combinatorial permutations of the five biomarker proteins. In one embodiment, the biomarker protein RCN-1 only is not selected for determination.

In some embodiment, the combinations of biomarker proteins selected for measurement can be ENO1 only, RCN-3 only, PEDF only, ENO 1 and PEDF, ENO 1 and RCN-3, ENO1 and α-SMA, ENO1 and RCN-1, RCN-1 and ENO1, PEDF and α-SMA, PEDF and RCN-3, PEDF and RCN-1, RCN-1 and RCN-3, α-SMA and ENO1, RCN-1 and α-SMA, (ENO1, RCN-3, and α-SMA), (ENO1, RCN-3, and PEDF), (ENO1, RCN-3, and RCN-1), (RCN-3, α-SMA, and PEDF), (RCN-3, α-SMA, and RCN-1), (RCN-3, α-SMA, and ENO1), (α-SMA, PEDF, and RCN-1), (α-SMA, PEDF, and RCN-3), (α-SMA, PEDF, and ENO1), (PEDF, RCN-1 and ENO1), (PEDF, RCN-1 and RCN-3), (PEDF, RCN-1 and α-SMA), (RCN-1, ENO1 and α-SMA), (ENO1, RCN-3, α-SMA, and PEDF), (RCN-3, α-SMA, PEDF, and RCN-1), (ENO1, RCN-3, α-SMA, and RCN-1), (ENO1, RCN-3, PEDF, and RCN-1), (ENO1, α-SMA, PEDF, and RCN-1) and all five biomarkers: ENO1, RCN-3, α-SMA, PEDF, and RCN-1.

In some embodiment, the combinations of biomarker proteins selected for measurement can further include additional biomarkers of Tables 4 and 5.

In one embodiment, the biomarker proteins are from a biological sample obtained from an individual and the biological sample is from an extracellular source. The extracellular source is selected from the group consisting of whole blood, plasma, serum, urine, bronco alveolar lavage, cerebrospinal fluid and spent culture media of ex vivo culture of a tissue excised from the individual. In another embodiment, the biological sample is from an intracellular source, such as a tissue excised from the individual, e.g. skin and lung tissues.

In some embodiments, the amount of biomarkers is measured by mass spectrometry, an antibody-based analytical method (e.g. Western blot or ELISA) or reverse transcriptase quantitative polymerase chain reaction (RT-qPCR).

In one embodiment, the reference amount is obtained from healthy individuals who do not have any active fibrotic condition described herein or from the average of a population of healthy individuals who do not have any fibrotic disorder. In another embodiment, the reference amount is obtained from healthy individuals who have not been diagnosed with any active fibrotic condition. The standard deviation described herein for prognosis evaluation is derived from the statistics of the amounts of the respective biomarker protein of a population of healthy individuals who do not have any fibrotic disorder.

In one embodiment, the individual is a mammal, e.g. a dog, cat, horse, cow etc; preferably the individual is a primate mammal such as a human.

Provided herein are devices and kits for measuring the biomarker protein amounts from a biological sample obtained from an individual.

### BRIEF DESCRIPTION OF THE DRAWINGS

FIG. 1 shows the common features between systemic sclerosis (SSc) and nephrogenic systemic fibrosis (NSF).

FIG. 2 shows the experimental approach to obtaining the secreted biomarker proteins from control, systemic sclerosis (SSc) and nephrogenic systemic fibrosis (NSF) fibroblasts.

FIG. 3 shows the two dimensional-difference gel electrophoresis (2D-DIGE). The supernatants from three SSc, three NSF and three normal fibroblast cultures were analyzed. The secretome from normal fibroblasts was conjugated with Cy2 fluorochrome, the secretome from SSc fibroblasts with Cy3 and the NSF secretome with Cy5.

FIG. 4A shows the differential analysis of SSc vs. NSF fibroblast secretomes showing 1403 spots (85%) with similar expression, 65 spots increased in the SSc sample and 226 spots increased in the NSF sample.

FIG. 4B shows the volume ratio analysis comparison of the secretome of normal fibroblasts with the secretome of SSc fibroblasts showing 450 spots increased (>two fold) in the SSc fibroblast secretome, 440 decreased (<50%) and 804 with similar expression.

FIG. 4C shows the differential analysis of NSF vs. normal fibroblast secretome showing 391 spots increased in NSF, 590 decreased and 713 with similar expression.

FIG. 5A shows exemplary data of a dimensional view displaying the intensity of fluorescence of a representative spot in different channels and their fluorescence intensity calculated by DIGE software. Three dimensional views displaying the intensity of fluorescence as volume of biomarkers selected by mass spectrometry analysis of the spots. The dark grey circle lines in the bottom panels indicate the spot boundaries obtained using the pixel data from images. The spot boundaries are utilized for spot volume calculations employing the DeCyder software

FIG. 5B shows a mass spectrometry exemplary data obtained for the two dimensional-2D-DIGE spot.

FIG. 6 shows the three dimensional view displaying the intensity of fluorescence as volume of biomarkers selected by mass spectrometry analysis of the spots that have the most upregulated in both SSc and NSF samples.

FIG. 7 shows the dot plots of fluorescence intensity ratio of the SSc (diamonds) and NSF (squares) secretomes both calculated against fluorescence values of normal secretome protein spots. The black dotted area indicates the spots (secreted biomarker proteins) with the highest increase in both SSc and NSF secretomes compared to normal secretome.

FIG. 8A shows tissue of expression of RCN-1 in skin biopsies from one SSc patient, one skin biopsy from an affected area of skin of a patient with NSF, and one normal skin biopsy analyzed by immunofluorescence followed by confocal microscopy showing substantially increased expression of RCN-1 in the dermis of the SSc and the NSF skin samples analyzed. The samples shown are representative of similar analyses of three SSc, three NSF and three normal skin samples.

FIG. 8B shows the quantitative analysis of fluorescence in the three SSc, three NSF and three normal skin biopsies showing the overall expression of RCN-1 expression in SSc skin biopsies to be 2.5 fold higher and in NSF skin biopsies to be 3.1 fold higher than that in normal skin. *=p<0.05 in two-tailed, unpaired T test.

FIG. 8C shows the Western blot showing that a band of protein recognized by the specific anti-RCN-1 antibody was clearly detectable in sera from the ten SSc patients examined and also of two control healthy individuals.

FIG. 9A (top view) and 9B (side view) show the schematic diagrams of an exemplary lateral flow immunoassay (LFIA) dipstick test strip for measuring that the level of a biomarker protein in a fluid sample is greater than (or is increased as compared to) a predetermined reference level.

FIG. 10A-D are schematic diagrams of the top views of exemplary LFIA dipstick test strips shown in FIG. 9, showing the different results that can obtained using such a simple test strip.

FIG. 11 shows a schematic diagram of how the levels of three biomarker proteins can be measured simultaneously using three independent LFIA test strips, one test strip for a different biomarker protein. A diagnostic kit can comprise several LFIA test strips, one strip for a different biomarker protein.

FIG. 12 shows a schematic diagram of how the levels of three biomarker proteins are measured simultaneously on the same LFIA test strip. A diagnostic kit can comprise a single composite or multiplex LFIA test strip for measuring the levels of several biomarker proteins simultaneously. The single composite test trip has three distinct protein binding agent specific respectively for three biomarker proteins.

FIG. 13A-D are schematic diagrams of an alternative embodiment of an exemplary LFIA dipstick test strip shown in FIG. 9 for measuring whether the level of a biomarker protein in a biological sample is above or below a reference/control value for that biomarker and the interpretation of the results obtained. Two different anti-biomarker antibodies or anti-biomarker protein-binding agent are used on the test strip.

FIG. 14A (top view) and 14B (side view) shows a schematic diagram of an alternative embodiment of a LFIA test strip for measuring the level of a biomarker protein in a biological sample and comparing the measured level with a reference value. S, T, C definition are as in FIG. 9.

FIG. 15A-F are schematic diagrams showing the different results that can obtained using the LFIA test strip shown in FIG. 14.

FIG. 16 shows a schematic diagram of an alternative version on how the levels of four biomarker proteins can be measured simultaneously using four separate LFIA test strips, one test strip for a different biomarker protein. A diagnostic kit can comprise multiple LFIA test strips, one strip for a different biomarker protein.

FIG. 17 shows a schematic diagram of an alternative version how the levels of three biomarker proteins are measured simultaneously on the same LFIA test strip. A diagnostic kit can comprise a single composite LFIA test strip for measuring the levels of several biomarker proteins.

FIG. 18 shows a schematic diagram of an ELISA plate assay comprising standard protein curves.

FIG. 19 shows a schematic diagram of a modified ELISA plate assay utilizing fixed amounts of standard proteins.

### DETAILED DESCRIPTION OF THE INVENTION

The inventors have discovered that fibroblast cells obtained from scleroderma tissues and/or fibroid associated with systemic sclerosis (SSc) and/or nephrogenic systemic fibrosis (NSF) secrete an abundance of certain proteins into the extracellular environment. These proteins are α-enolase (ENO1), reticulocalbin 3 (RCN-3), alpha smooth muscle actin (α-SMA), reticulocalbin 1 (RCN-1), tropomyosin 4 (TMP4), alpha actin 1 (ACTA1), calreticulin (CALR) and pigment epithelium-derived factor (PEDF) and others shown in Tables 4 and 5. Under normal healthy conditions, these most proteins are not found in great abundance extracellularly. Therefore, an increase in the amount of select proteins in the extracellular space can be used as biomarkers for early diagnosis of SSc, NSF or other chronic fibrotic diseases, and for the prognostic evaluation of SSc, NSF or other chronic fibrotic diseases during a treatment regime.

A hallmark of SSc, NSF or other chronic fibrotic conditions is the excess production of extracellular matrices (ECM), mainly in the form of collagen, by affected fibroblast cells. The present invention provides a method for determining the likelihood, the early diagnosis and the prognosis evaluation of an active fibrotic condition such as SSc, NSF or other chronic fibrotic conditions by measuring the amount of RCN-1 (reticulocalbin 1) (alias RCN; RCAL; PIG20; F1137041), RCN-3 (reticulocalbin 3) (alias RLP49), α-SMA (alpha smooth muscle actin), PEDF (pigment epithelium-derived factor) (alias SERPINF1, serpin peptidase inhibitor, clade F, proliferation factor 35), and ENO1 (α-enolase) (alias NNE; PPH; MPB1; MBP-1; ENO1L1) as the five proteins that are preferably used as biomarkers. It is also contemplated that other proteins disclosed in Tables 4 and 5 are used as biomarker proteins too, preferably in combination with ENO1, RCN-3, RCN-1, PEDF, and α-SMA.

As used herein, the term “biomarker” and “biomarker protein” are used interchangeably.

Accordingly, one embodiment of the invention provides a method for determining the likelihood of an individual having SSc, NSF or other chronic fibrotic diseases comprising: (a) measuring an amount of at least one selected from the group consisting of ENO1, RCN-3, and PEDF, wherein the at least one biomarker protein is selected alone or in combination with biomarker proteins RCN-1 and/or α-SMA; and (b) comparing the amount of step (a) with a reference amount, wherein the amount of step (a) is greater than the reference amount indicates that the individual has an increased likelihood of having an active fibrotic condition. This method is used for an early diagnosis of an individual on whether the individual is having an active fibrotic condition such as SSc, NSF or other chronic fibrotic disease. The biomarker proteins are found in a biological sample for the individual.

In one embodiment, provided herein is a method of determining the likelihood of an individual having SSc, NSF or other chronic fibrotic diseases comprising contacting a biological sample obtained from an individual with at least an agent that binds a biomarker selected from group consisting of ENO1, RCN-3, and PEDF, wherein the at least one biomarker protein is selected alone or in combination with biomarker proteins RCN-1 and/or α-SMA, wherein an increase of the biomarker over that of a reference amount indicates that the individual has an increased likelihood of having an active fibrotic condition. The individual is preferably a human. The agent is a protein-binding agent. The increase of the biomarker over that of a reference amount is at least two standard deviations. The contacting of the biological sample and the agent produces a complex formed between the biomarker in the biological sample and the agent.

In some embodiments, various combinations of the biomarker proteins can be selected for analysis. For examples, combinations can include ENO1 only, RCN-3 only, PEDF only, ENO 1 and PEDF, ENO 1 and RCN-3, ENO1 and α-SMA, ENO1 and RCN-1, RCN-1 and ENO1, PEDF and α-SMA, PEDF and RCN-3, PEDF and RCN-1, RCN-1 and RCN-3, α-SMA and ENO1, RCN-1 and α-SMA, (ENO1, RCN-3, and α-SMA), (ENO1, RCN-3, and PEDF), (ENO1, RCN-3, and RCN-1), (RCN-3, α-SMA, and PEDF), (RCN-3, α-SMA, and RCN-1), (RCN-3, α-SMA, and ENO1), (α-SMA, PEDF, and RCN-1), (α-SMA, PEDF, and RCN-3), (α-SMA, PEDF, and ENO1), (PEDF, RCN-1 and ENO1), (PEDF, RCN-1 and RCN-3), (PEDF, RCN-1 and α-SMA), (RCN-1, ENO1 and α-SMA), (ENO1, RCN-3, α-SMA, and PEDF), (RCN-3, α-SMA, PEDF, and RCN-1), (ENO1, RCN-3, α-SMA, and RCN-1), (ENO1, RCN-3, PEDF, and RCN-1), (ENO1, α-SMA, PEDF, and RCN-1) and all five biomarkers ENO1, RCN-3, α-SMA, PEDF, and RCN-1.

In one embodiment, the biomarker protein RCN-1 only is not selected for the methods described herein. In one embodiment, the biomarker protein α-SMA only is not selected for the methods described herein. In one embodiment, the biomarker proteins RCN-1 and/or α-SMA are selected in combination with another biomarker proteins listed in Tables 4 and 5 for the methods described herein, preferably ENO1, RCN-3 and PEDF.

In one embodiment, the active fibrotic condition is selected from the group consisting of scleroderma, nephrogenic systemic fibrosis, fasciitis, scleromyxedema, retroperitoneal fibrosis, pulmonary fibrosis, liver fibrosis, chronic graft versus host disease and chronic allograft rejection. In other embodiments, the active fibrotic condition includes but is not limited to rheumatoid arthritis, lupus, autoimmune diseases, Lyme disease, asthma, idiopathic pulmonary fibrosis, chronic pulmonary fibrosis, uterine fibrosis, and ovarian fibrosis.

As used here in, in one embodiment, the term “early diagnosis” is used with respect to an individual who is suspected of suffering SSc, the individual can have some clinical symptoms that are known to be associated with a fibrotic condition but have not been differentially and definitively been diagnosed with a fibrotic condition. In another embodiment, the term “early diagnosis” is used with respect to an individual who is asymptomatic, i.e. has no physically obvious clinical symptoms that are known to be associated with a fibrotic condition. In another embodiment, the term “early diagnosis” is used with respect to an individual who is asymptomatic, but is at risk of developing a fibrotic condition such as SSc, NSF or other chronic fibrotic diseases.

In one embodiment, the individual needing early diagnosis is suspected of suffering SSc, NSF or other chronic fibrotic disease and is therefore in need of a diagnosis according to the method described herein. The individual can be exhibiting clinical symptoms such as insidious swelling of the distal extremities with gradual thickening of the skin of the fingers. The swelling and thickness can spread to the hands and/or other parts of the body. The skin can becomes taut, shiny, and hypopigmented or hyperpigmented; the face can became masklike; and telangiectases can appear on the fingers, chest, face, lips, and tongue. Subcutaneous calcifications can develop, usually on the fingertips (pulps) and over bony eminences. The individual can have trophic ulcers, especially on the fingertips, overlying the finger joints, or over calcinotic nodules. Abnormal capillary and microvascular loops in the nails can also be present.

In another embodiment, the individual needing early diagnosis is asymptomatic, i.e. has no physically obvious clinical symptoms such as cutaneous symptoms described herein that are known to be associated with SSc, NSF or other chronic fibrotic diseases as described herein or cutaneous symptoms described in the modified Rodnan skin score (mRSS) which in the current method of assessing the SSc. The early diagnostic method described herein can be incorporated into a routine physical examination workup. In another embodiment, the individual is asymptomatic, but is at risk of developing SSc, NSF or other chronic fibrotic diseases. For example, the individual has kidney disease, has kidney insufficiency and had a recent MRI performed, suffers from an autoimmune disease such as mixed connective tissue disease, has idiopathic pulmonary hypertension or gastrointestinal abnormalities, or has family members suffering from or who was previously diagnosed with SSc, NSF or other chronic fibrotic diseases.

In one embodiment, the individual is a mammal, e.g. a dog, cat, horse, cow etc.; preferably the individual is a primate mammal such as a human.

In one embodiment, early diagnosis is performed before the individual has developed any obvious and potentially harmful and limiting skin sclerosis. Since SSc and NSF are multisystem, multistage diseases marked by variable clinical symptoms, absence of cutaneous involvement does not exclude the diagnosis of these diseases. Early diagnosis afforded by the method described herein can influence treatment options and thus greatly influence long term prognosis and mortality of individuals affected by these chronic diseases and mortality.

In one embodiment, an early diagnosis is performed when the individual has tested positive for anticentromere (ACA), anti-topoisomerase-1 and/or anti-Scl-70 antibodies. The individual does not have any obvious skin sclerosis or does not have limited cutaneous systemic sclerosis (lcSSc). The presence of ACA and anti-Scl-70 antibodies has been described in patients with SSc. ACAs are found in roughly 80-90% of patients with lcSSc and only rarely in patients with diffuse cutaneous systemic sclerosis (dcSSc). Antibodies to topoisomerase-1 are present in 40% of patients with dcSSc. The presence of either ACAs or anti-Scl-70 is highly specific (95% to 99%) for the diagnosis of lcSSc and dcSSc, respectively. The antibodies are present only rarely in healthy subjects and patients with other rheumatologic disease. The presence of ACA or anti-Scl-70 antibodies is also highly specific for underlying SSc in patients presenting initially with isolated Raynaud's phenomenon (RP) and therefore can be helpful in the evaluation of RP.

In some embodiments, as an exemplary situation, a physician can prescribe the early diagnosis method as described herein to a patient with or without cutaneous sclerosis, when the physician suspects that the patient might be afflicted and/or the physician wants to rule out the possibility that the patient might be afflicted. A biological sample is obtained from the patient; e.g. the patient's blood is drawn by the physician or a skilled technician. The plasma or the serum is then separated from the blood cells in the sample of blood, and the levels of the biomarker proteins ENO1, RCN-3 or PEDF alone or in combination with RCN-1 and/or α-SMA or biomarkers listed in Tables 4 and 5, are measured from the plasma or serum. The levels of the biomarker proteins can be measured by any methods known in the art, such as, antibody-based assays such as enzyme-linked immunoabsorbant assay (ELISA) and Western blot or mass spectroscopy.

The level of each biomarker protein from the patient's plasma is compared to the level of the respective biomarker protein from the plasma of a healthy individual or to the average level of the respective biomarker protein obtained the plasmas of a population of healthy individuals. When the levels of the biomarker proteins from the patient is higher than the control reference levels, patient has an increased likelihood of having an active fibrotic condition such as scleroderma, nephrogenic systemic fibrosis, fasciitis, scleromyxedema, retroperitoneal fibrosis, pulmonary fibrosis, liver fibrosis, chronic graft versus host disease and chronic allograft rejection. In one embodiment, when the level of each of the measured biomarker proteins from the patient is at least two standard deviations greater than the average reference amount obtained for healthy individuals, it indicates that the patient has a likelihood of having a fibrotic condition. In some embodiments, the level of each of the measured biomarker proteins from the patient is between two-ten standard deviations greater than the average reference amount obtained for healthy individuals, e.g. three, four, five, six, seven, eight, nine or ten standard deviations greater than the average reference amount obtained for healthy individuals.

In one embodiment, the amount of at least one of biomarker proteins ENO1, RCN-3, α-SMA or PEDF alone or in combination with RCN-1 is at least two standard deviations greater than the reference amount. For example, if the normal average and standard deviation of ENO1 levels in the plasma in the normal healthy human population is 3.1±0.3 pmol/ml and for PEDF level is 4.38±0.5 μg/ml, then any amount of plasma ENO1 of more than 3.7 pmol/ml and PEDF more than 5.38 μg/ml indicates the likelihood of that individual have an active fibrotic condition. Furthermore, an increase in the same patient of the levels of one or more biomarker proteins ENO1, RCN-3, α-SMA or PEDF alone or in combination with RCN-1 from a previous earlier time point measurement can be predictive of disease progression or relapse. The clinician can then decide to perform additional tests to confirm the early diagnosis and prescribe a treatment plan.

In one embodiment, the reference amount is an average amount from a population of healthy individuals who have not been diagnosed with an active fibrotic condition. The average levels of the respective biomarker proteins from a population of healthy individuals represent the reference or control levels. Also from data obtained for the population of healthy individuals, one skilled in the art would be able to calculate the average and standard deviation for each biomarker protein in a healthy individual. A population of healthy individuals comprises at least five or more individuals for the purpose of calculating the average and standard deviation for each biomarker protein. The levels of the biomarkers of these healthy individuals are averaged. In one embodiment, the average ENO1 level in the serum or plasma of healthy individuals is less than 1 μg/ml. In one embodiment, the average plasma PEDF level in 33 healthy individuals is 4.38±0.59 μg/ml. The plasma PEDF level increases with age to about 6.12 μg/ml.

In one embodiment, the population of healthy individuals are grouped by age and gender and the average amount of the respective biomarker protein are thus obtained for the age/gender groups, e.g. averages for populations of individuals in their teen, 20 s, 30 s, 40 s, 50 s, 60 s, 70 s, 80 s, 90 s and 100, and male and females. In using the methods described herein, the age/gender matched averages and respective standard deviations are used.

To Calculate Mean or Average of N number healthy individuals (N≧5):

$$Average = \frac{\begin{matrix}Sum of the levels of one biomarker \\ protein from N healthy individuals\end{matrix}}{N number of healthy individuals}$$

To Calculate Standard Deviation:

1. Collect biomarker protein levels from at least 5 healthy individuals

2. Get the Mean (average): sum of levels divided by number of individuals

3. Get the deviations: subtract the mean from each of the collected protein levels in step 1

4. Square the deviations

5. Add the squares

6. Divide by total number of individuals less one

7. Square root of result is standard deviation

In one embodiment, the biomarker proteins are from a biological sample obtained for an individual and the biological sample is from an extracellular source. In one embodiment, the extracellular source is selected from the group consisting of whole blood, plasma, serum, urine, saliva, bronco alveolar lavage, cerebrospinal fluid and spent culture media of ex vivo culture of a tissue excised from the individual. A skilled clinician, medical technician or surgeon in the art would be able to obtain whole blood from the peripheral circulation of an individual and/or obtained a tissue biopsy from the individual. From the whole blood, a skilled clinician or technician in the art would be able to obtain the plasma or serum. Methods of primary fibroblast cell culture are well known in the art, for example, as described in Paw and Zon (Methods Cell Biol. 1999, 59:39-43) in Tamm, M. et al., Transplantation, 2002, 71:337-339) and Kurtycz et al., (Ped. Path. & Lab Med, 1998, 18:35-39). The spent culture media of the primary fibroblast cell culture is collected, clarified by centrifugation at 10,000×G for 30 min and then processed for determination and quantification of the biomarker proteins ENO1, RCN-3, α-SMA, RCN-1 or PEDF by any methods known in the art, such as mass spectrometry (MS) ELISA or Western blot.

In one embodiment, the measurement of the amount of at least one of biomarker proteins ENO1, RCN-3, α-SMA, or PEDF alone or in combination with RCN-1 is performed by MS or an antibody-based analytical method such as ELISA or Western blot.

In one embodiment, the biomarker proteins are from a biological sample obtained for an individual. The biological sample is from an intracellular source. In one embodiment, the intracellular source is a tissue excised from the individual. A skilled clinician, medical technician or surgeon in the art would be able to obtain a tissue biopsy from the individual, for example, a skin biopsy. The tissue excised can be prepared for the measurement of levels of the biomarker proteins ENO1, RCN-3, or PEDF alone or in combination with RCN-1 and/or α-SMA, for example, for antibody-based Western blot analysis or for RT-qPCR.

In one embodiment, the biomarkers are from excised tissues is performed by mass spectrometry, an antibody-based analytical method or quantitative-RT-PCR.

In one embodiment, the invention also provides a method of prognosis evaluation in an individual diagnosed with and is being treated for an active fibrotic condition, such as, SSc, NSF or other chronic fibrotic diseases, the method comprising: (a) at a first time point, measuring the amount of at least one biomarker protein selected from the group consisting of ENO1, RCN-3, RCN-1 and PEDF, wherein the measurement is not RCN-1 only and not α-SMA only, wherein the biomarker is from a biological sample obtained form the individual; (b) at a second time point, measuring the amount of at least one biomarker protein selected from the group consisting of ENO1, RCN-3, RCN-1 and PEDF, wherein the measurement is not RCN-1 only and not α-SMA only, wherein the biomarker is from a biological sample obtained form the individual, wherein the second time point is after the first time point, and wherein a combination of biomarker proteins measured is the same at both first and second time points; and (c) comparing the amount of step (a) with the amount of step (b), wherein the amount of step (b) is less than the amount of step (a) indicates that the treatment is effective in the individual. This method provides an easy and fast way of monitoring the efficacy of an SSc, NSF or other chronic fibrotic diseases therapy in the afflicted individual having the active fibrotic condition. The measurement method is same for both the first and second time points. The biological samples are also the same for both the first and second time points.

In one embodiment, the method of prognosis evaluation is performed after commencing treatment with a therapy for an active fibrotic condition described herein. The levels of biomarker proteins can be monitored at regular periods during the course of therapy, for example, every two months. The levels of biomarker proteins measured at any given time are compared to the levels of biomarker proteins obtained in the last measurement or a measurement at an earlier time point. In one embodiment, the levels of biomarker proteins in the second time point (step b) is more than two standard deviations lower than the levels of biomarker proteins determined in the first time point (step a), this means that the levels of biomarker proteins measured in the second time point are statistically significantly less than the levels of biomarker proteins measured in the first time point. Such levels are indicative that the therapy is effective.

As used herein, the term “statistically significantly” based on a minimum of two standard deviations from the average refers to a statistical confidence level of 95% that the indication in accurate.

In another embodiment, during the course of therapy, when the levels of biomarker proteins in the second time point (step b) are statistically significantly greater or unchanged compared to the levels of biomarker proteins measured in the first time point (step c) or a measurement at an earlier time point, it is indicative that the therapy is ineffective or has become ineffective and the individual continues to have active fibrosis conditions described herein.

In yet another embodiment, the method of prognosis evaluation is performed after the completion or termination of treatment with a therapy for an active fibrotic condition described herein, e.g. SSc, NSF or other chronic fibrotic diseases. The patient had been successfully treated for the condition, is currently symptom free and had normal levels of the biomarkers described herein. The method can be used to monitor for recurrence of any active fibrotic condition. In this embodiment, when the levels of biomarker proteins in the second time point (step b) are more than two standard deviations higher than the levels of biomarker proteins measured in the first time point (step a), this means that the levels are statistically significantly greater than the levels of biomarker proteins measured in the first time point and/or an earlier time point, it is indicative that the patient has a likelihood of recurrence of an active fibrotic condition described herein. The first time point (step a) and/or an earlier time point is when the patient was symptom free.

In one embodiment, provided herein is a device comprising: (a) at least one protein-binding agent which specifically binds to at least one biomarker protein selected from the group of ENO1, RCN-3, and PEDF, and at least one protein-binding agent which specifically binds to RCN-1 and/or α-SMA; and (b) at least one solid support for the at least one protein-binding agent in step (a), wherein the protein-binding agent is deposited on the solid support.

In another embodiment, provided herein is a kit comprising: (a) a device comprising: at least one protein-binding agent which specifically binds to at least one biomarker protein selected from the group of ENO1, RCN-3, and PEDF, and at least one protein-binding agent which specifically binds to RCN-1 and/or α-SMA; and at least one solid support for the at least one protein-binding agent, wherein the protein-binding agent is deposited on the solid support; and (b) a first agent, wherein the first agent produces a detectable signal in the presence of a protein-binding agent which deposited on the device is specifically bound to a biomarker protein selected from the group of: ENO1, RCN-3, α-SMA, RCN-1 and PEDF.

In one embodiment, the kit further comprises a second agent, wherein the second agent produces a different detectable signal in the presence of a second protein-binding agent deposited on the device which is specifically bound to a second biomarker protein selected from the group of ENO1, RCN-3, α-SMA, RCN-1 and PEDF, wherein the first and second biomarker are not the same.

In some embodiments, the protein-binding agent deposited on the solid support specifically binds to the biomarker protein of ENO1 of SEQ ID NO: 3, the biomarker protein of RCN-3 of SEQ ID NO: 4, the biomarker protein of PEDF of SEQ ID NO: 2, the biomarker protein of RCN-1 of SEQ ID NO: 1 and the biomarker protein of α-SMA of SEQ ID NO: 5. The protein-binding agent deposited on the device specifically binds to the biomarker protein when the level of the biomarker protein is at least two standard deviations above a reference level for that biomarker protein.

In some embodiment, the protein-binding agent is an antibody, an antibody-binding moiety, antibody fragment, aptamer, small molecule or variant thereof.

In one embodiment, the solid support is in the format of a dipstick, a microfluidic chip or a cartridge.

### Systemic Sclerosis

Systemic sclerosis (SSc) is a rare disorder, with an annual incidence in the United States of 19.3 cases per million adults. Several studies have estimated the prevalence of SSc in the United States to be around 250 cases per million adults. International reports from Britain and Japan report a lower prevalence, of around 35 cases per million persons. Women are roughly four times more likely than men to develop SSc. In addition, blacks are more commonly affected than whites and are at greater risk for diffuse disease. Most patients with SSc present after the third or fourth decade of life.

Scleroderma is a disease that causes thickened skin and varying degrees of organ dysfunction resulting from small-vessel vasculopathy and immune-mediated fibrosis. The clinical manifestations of this disease are extremely heterogeneous and depend on the presence and degree of internal organ involvement. Patients can present with a spectrum of illness ranging from localized skin fibrosis only (localized scleroderma) to a systemic disorder (SSc) with both cutaneous and internal organ involvement.

Localized scleroderma includes various forms of cutaneous sclerosis without internal organ involvement (Table 1). These forms of scleroderma can be disfiguring but only rarely require systemic therapy to control disease activity.

Systemic sclerosis is further divided into two subsets of disease, depending on the degree of skin and organ involvement (Table 1). The presence of dcSSc denotes the presence of extensive cutaneous sclerosis over the proximal limbs, trunk, and face. Patients with lcSSc have fibrosis limited to the hands, forearms, feet, legs and face. Both dcSSc and lcSSc are associated with internal organ involvement; however, patients with dcSSc are at greater risk of clinically significant major organ dysfunction. Some patients with lcSSc may be further classified as having the CREST syndrome, with accompanying calcinosis, Raynaud's phenomenon (RP), esophageal dysmotility, sclerodactyl), and cutaneous telangiectasias. Scleroderma sine sclerosis is a rare disorder in which patients develop vascular and fibrotic damage to internal organs in the absence of cutaneous sclerosis.

Fibrosis is the accumulation of excessive connective tissue. The main cells of connective tissue are fibroblast. They manufacture and maintain the extracellular material. Fibroblasts migrate throughout the ECM wherever they are needed, such as in scar formation. Fibroblasts contribute to the formation of (1) the areolar (or loose) connective tissue that holds organs and epithelia in place, and has a variety of proteinaceous fibres, including collagen and elastin; and to the formation of (2) the fibrous connective tissue forms ligaments and tendons. Its densely packed collagen fibers have great tensile strength. Blood functions in the transport of dissolved nutrients, hormones, and carbon dioxide in the form of bicarbonate.

Pathophysiology of SSc involves vascular damage and activation of fibroblasts; and collagen and other extracellular proteins in various tissues are overproduced. Scleroderma is characterized by immune system activation, endothelial dysfunction, and enhanced fibroblast activity. The precise inciting events leading to the development of SSc are currently unknown. Most experts believe that the effector cell is the activated fibroblast. Several cytokines including interleukin-4 and transforming growth factor-beta (TGF-β) have been implicated in fibroblast activation in patients with scleroderma. These cytokines are released from activated immune cells, fibroblasts, and endothelial cells. Activated fibroblasts elaborate structurally normal collagen and other extracellular matrix proteins in the skin and various internal organs.

Vascular injury occurs even before clinically obvious fibrosis; indeed, RP is often the earliest clinical finding in patients who eventually develop SSc. Most damage occurs at the level of the cutaneous circulation and in the microvasculature of various internal organs. The vascular injury is believed to originate at the level of the endothelial cells. Small arteries and capillaries constrict, with eventual obliteration of the vessel lumen and ensuing ischemia.

Clinical manifestations of SSc are heterogeneous and vary as a result of type of disease (limited or diffuse) and organ involvement (Table 2). Patients with dcSSc are at risk for rapidly progressive skin fibrosis and widespread, severe, internal organ involvement. Patients with lcSSc have a disease course characterized by slowly progressive skin changes not extending beyond the elbows and knees into the proximal extremities or trunk, along with varying degrees of internal organ involvement.

RP is present in most patients with SSc and is often the earliest manifestation of disease. RP can be present for years before clinically significant skin changes or internal organ involvement develops. Although some patients with RP may not develop the entire spectrum of Raynaud skin changes, most will have digital pallor in response to cold or stress.

Cutaneous changes usually begin with an early phase of skin edema, manifested as swollen fingers and hands. In dcSSc, these changes are followed by the development of firm, thickened skin over the extremities, trunk, face, and hands. The patients in whom these changes develop more rapidly are at greater risk for serious internal organ involvement such as pulmonary fibrosis and renal failure. Skin changes typically peak before the first 5 years. As a result of skin thickening, flexion contractures can develop over joints. Skin thickening may then begin to regress, as manifestations of internal organ involvement become more clinically evident. In patients with lcSSc, early skin changes include RP and digital ulceration. Skin thickening and digital edema are confined to the distal extremities (beyond the elbows and the knees). As the disease progresses, there is an increase in cutaneous telangectasias, calcinosis, and digital ischemia.

Patients may complain of dyspnea or nonproductive cough as a manifestation of underlying pulmonary disease. Some patients may be asymptomatic but manifest changes on chest radiography (lower-lobe interstitial infiltrates) or physical examination (basilar rales). Lower-extremity edema and other signs or symptoms of right-sided heart failure may also lead to suspicion of pulmonary hypertension. Pulmonary function tests are frequently abnormal, revealing restrictive changes even in the absence of radiographic changes or exertional dyspnea. Patients with dcSSc are more likely to develop interstitial lung disease, and a subset of patients with lcSSc can develop a severe form of pulmonary arterial hypertension without interstitial lung disease. In patients with either form of SSc, symptomatic pulmonary disease is a relatively uncommon finding in the first few years of disease. However, patients with dcSSc and rapidly progressive skin changes or anti-topoisomerase antibodies (anti-Scl-70) are at risk for earlier onset of severe pulmonary disease.

Scleroderma renal crisis is characterized by the development of severe hypertension, renal insufficiency, and microangiopathic hemolytic anemia. A small subset of patients may be normotensive at diagnosis. Patients with rapidly progressive diffuse skin fibrosis, users of glucocorticoids, or those with large, chronic pericardial effusions are at greatest risk. The development of renal crisis is rare in patients with lcSSc, and most cases occur in the first few years of dcSSc. Renal failure can follow a rapidly progressive course, and early recognition and treatment with angiotensin-converting enzyme inhibitors is critical.

Gastrointestinal involvement is common in both forms of SSc. Atrophy of the muscularis mucosa and submucosal fibrosis result in varying degrees of esophageal and other gastrointestinal dysfunction. Complaints of dysphagia and heartburn are common and often signal the development of esophageal dysmotility. Esophageal disease can progress, resulting in peptic esophagitis, esophageal strictures, and eventual development of an atonic esophagus. Gastric telangiectasia (“watermelon stomach”) can lead to chronic upper gastrointestinal bleeding and iron deficiency anemia. Small-bowel motility may also be affected, resulting in varying degrees of malabsorption and bacterial overgrowth. Severe constipation may develop from colonic hypomotility. Gastrointestinal bleeding is infrequent, but may occur from erosive esophagitis, “watermelon stomach”, wide-mouth diverticula in the colon, and gastritis. Pneumatosis cystoides intestinalis can present as an abdomen. Patients with lcSSc may also develop primary biliary cirrhosis.

Cardiac involvement is rare in lcSSc, but myocardial fibrosis may develop in patients with dcSSc, resulting in heart failure, arrhythmias, and atrioventricular conduction defects. Other features of SSc may include dry eyes and mouth (secondary to Sjögren's syndrome) and hypothyroidism (Hashimoto's thyroiditis). Musculoskeletal involvement is common and may present as nonspecific myalgias and arthralgias or true arthritis. Muscle weakness may also occur in the presence of muscel inflammation (myositis) with elevated serum levels of muscle enzymes.

The diagnosis of SSc is usually made on the basis of the characteristic cutaneous finding of skin thickening in association with RP and various degrees of internal organ involvement. In early disease, RP may be the dominant cutaneous finding. Examination of the nailfold capillaries with capillaroscopy may be helpful in measuring whether RP is due to primary disease or secondary to a systemic disorder such as scleroderma. Other disorders associated with scleroderma-like skin changes, such as eosinophilic fasciitis and eosinophilia-myalgia syndrome, are not typically associated with RP.

Diagnostic criteria proposed by the American College of Rheumatology (formerly known as the American Rheumatism Association) are shown in Table 3.

### Nephrogenic Systemic Fibrosis

Nephrogenic systemic fibrosis (NSF), described in 2000, is an emerging systemic disorder characterized by widespread tissue fibrosis. NSF was not known to the medical community before March 1997. It occurred only in people with kidney insufficiency. Neither the duration of kidney disease nor its underlying cause are related to the development of NSF. Some patients with NSF develop skin tightening in the earliest stages of kidney disease, and others may have had kidney disease for years. Specific triggers for the development of NSF are still being investigated. There is no convincing evidence that NSF is caused by a medication, a microorganism, or by dialysis. Recent reports have strongly correlated the development of NSF with exposure to gadolinium-containing MRI contrast agents. At this point it appears NSF is a systemic disorder with its most prominent and visible effects in the skin. For this reason, NSF has been suggested as an equivalent terminology in those previously diagnosed with nephrogenic fibrosing dermopathy (NFD), and is preferred in that it more accurately reflects the current understanding of the disorder.

NSF appears to affect males and females in approximately equal numbers. NSF has been confirmed in children and the elderly, but tends to affect the middle-aged most commonly. It has been identified in patients from a variety of ethnic backgrounds and from North America, Europe, and Asia.

Besides kidney disease, conditions that may be associated with NSF include coagulation abnormalities and deep venous thrombosis, recent surgery (particularly vascular surgery), recent failure of a transplanted kidney, and sudden onset kidney disease with severe swelling of the extremities. It is very common for the NSF patient to have undergone a vascular surgical procedure (such as revision of an AV fistula, or angioplasty of a blood vessel) or to have experienced a thrombotic episode (thrombotic loss of a transplant or deep venous thrombosis) approximately two weeks before the onset of the skin changes.

Symptoms and signs of NSF include tightening and swelling of the skin, typically starting with the legs, moving to the arms, and sometimes the trunk; thickening of the skin around the joints, restricting movement; skin which feels “woody” and has a texture similar to that of an orange peel; red or dark patches of skin; burning, itching, and/or sharp pains in affected areas; fluctuating hypertension preceding the appearance of skin lesions; symmetrical skin lesions, commonly on the ankles and thighs and between the wrists and upper arms; muscle weakness; deep bone pain in the hips and ribs; calcification of soft tissues; and yellow plaques near the eyes.

Currently, there is no consistently successful treatment for NSF. Improving renal function, due to any modality, seems to slow or arrest NSF, and in many cases allows for gradual reversal of the process over time.

As NSF is a rare, relatively recent diagnosis, the natural history of the disease is not well understood. Some patients report a gradual improvement in mobility and slight softening of the skin over time. Complete spontaneous healing in a patient with ongoing kidney disease has not yet been reported.

Several patients with NSF have died as a result of complications of their kidney disease or transplant surgery. One patient, who elected to discontinue dialysis, had widespread fibrosis involving the diaphragm, psoas muscles, proximal esophagus and intimal areas of vessels of the kidney and lungs.

Some patients with NSF (estimated at 5% or less) have an exceedingly rapid and fulminant disease course that can result in death. NSF, by itself, is not a cause of death, but may contribute to death by restricting effective ventilation, or by restricting mobility to the point of causing an accidental fall that may be further exacerbated by fractures and clotting complications.

### DEFINITIONS OF TERMS

As used herein, the term “afflicted” use herein refers to suffering from an “active fibrotic condition” such as SSc a.k.a. scleroderma, NSF, fasciitis, scleromyxedema, retroperitoneal fibrosis, pulmonary fibrosis, liver fibrosis, chronic graft versus host disease and chronic allograft rejection.

The term “standard deviation” is a measure of the dispersion of a collection of numbers. For example, the standard deviation for the average normal level of protein biomarker ENO1 is the dispersion of a collection of the of ENO1 amounts found in the extracellular source or intracellular source of a population of healthy individuals that have not been diagnosed with any active fibrotic condition such as SSF, NSF, fasciitis, scleromyxedema, retroperitoneal fibrosis, pulmonary fibrosis, liver fibrosis, chronic graft versus host disease or chronic allograft rejection.

As used herein, the terms “amount” and “level” of a protein biomarker are used interchangeably.

As used herein, the term “active fibrotic condition” refers to any condition in which fibrotic tissue, scar tissue, connective tissue, and/or extracellular matrix (ECM) material accumulates on or within one or more organs within the body in response to tissue injury (e.g., infection, autoimmune reaction, mechanical injury etc.) or for unknown etiology and that accumulation of tissues and ECM in currently in progress. The condition is occurring at the present time. As used herein, the expression “fibrosis conditions” and the expression “fibrotic conditions” are intended to have the same meaning. Specific examples of include but not limited to SSc or scleroderma, NSF, fasciitis, scleromyxedema, retroperitoneal fibrosis, pulmonary fibrosis, liver fibrosis, chronic graft versus host disease and chronic allograft rejection.

As used herein, the term “comprising” means that other elements can also be present in addition to the defined elements presented. The use of “comprising” indicates inclusion rather than limitation.

The term “consisting of” refers to methods as described herein, which are exclusive of any element not recited in that description of the embodiment.

As used herein, the term “RT-qPCT” refers to real-time quantitative reverse transcriptase polymerase chain reaction (RT-qPCR) which is a method of reliable detection and measurement of products generated during each cycle of the PCR process which are directly proportionate to the amount of messenger RNA prior to the start of the PCR process.

The term “treat” or “treatment” refer to both therapeutic treatment wherein the objective is slow down, and/or halt the development or progression of any active fibrotic condition such as SSc, NSF, fasciitis, scleromyxedema, retroperitoneal fibrosis, pulmonary fibrosis, liver fibrosis, chronic graft versus host disease and chronic allograft rejection. “Treatment” can mean a reduction in the amount of secreted ECM and/or scar tissue formation. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. In one embodiment, “treatment” refers to prophylactic or preventative measures to prevent recurrence of the fibrotic condition after the subject has been successfully treated for the active condition and is currently symptom free.

As used herein, the terms “reference amount”, “reference level” or “control level” all refer to the level of a biomarker protein from a healthy individual or to the average level of a biomarker protein calculated from the biomarker data collected from a population of healthy individuals.

As used herein, the term “healthy individual” is a human or a mammal who does not have and has not been diagnosed with any active fibrotic condition such as SSc, NSF, fasciitis, scleromyxedema, retroperitoneal fibrosis, pulmonary fibrosis, liver fibrosis, chronic graft versus host disease or chronic allograft rejection.

As used herein, the term “extracellular” means refers to the surrounding area external of a cell.

An “extracellular” source would be materials found in the surrounding area external of a cell, such as, the plasma, serum, urine, cerebrospinal fluid and the extracellular matrix.

As used herein, the term “intracellular” means refers to the material inside of a cell. An “intracellular” source would be any materials derived found inside a cell, such as, the cytoplasm of a cell, cell lysate, and total RNA preparation.

Unless otherwise explained, all technical and scientific terms used herein have the same meaning as commonly understood by one of ordinary skill in the art to which this disclosure belongs. Definitions of common terms in dermatology, rheumatology, biochemistry and molecular biology can be found in The Merck Manual of Diagnosis and Therapy, 18th Edition, published by Merck Research Laboratories, 2006 (ISBN 0-911910-18-2); Robert S. Porter et al. (eds.), The ELISA guidebook (Methods in molecular biology 149) by Crowther J. R. 2000; Fundamentals of RIA and Other Ligand Assays by Jeffrey Travis, 1979, Scientific Newsletters; Immunology by Werner Luttmann, published by Elsevier, 2006; in Benjamin Lewin, Genes IX, published by Jones & Bartlett Publishing, 2007 (ISBN-13: 9780763740634); Kendrew et al. (eds.), and The Encyclopedia of Molecular Biology, published by Blackwell Science Ltd., 1994 (ISBN 0-632-02182-9).

Unless otherwise stated, the present invention was performed using standard procedures, as described, for example in Maniatis et al., Molecular Cloning: A Laboratory Manual, Cold Spring Harbor Laboratory Press, Cold Spring Harbor, N.Y., USA, 1982; Sambrook et al., Molecular Cloning: A Laboratory Manual (2 ed.), Cold Spring Harbor Laboratory Press, Cold Spring Harbor, N.Y., USA, 1989; Current Protocols in Molecular Biology (CPMB) Ausubel, F. M., et al. (ed.), John Wiley and Sons, Inc.; Current Protocols in Protein Science (CPPS) Coligan, J. E., et. al., (ed.), John Wiley and Sons, Inc.; Current Protocols in Immunology (CPI) Coligan, J. E., et. al., (ed.) John Wiley and Sons, Inc.; Current Protocols in Cell Biology (CPCB) Bonifacino, J. S., et. al. ed.), John Wiley and Sons, Inc.; Freshney R. I., Culture of Animal Cells: A Manual of Basic Technique, Publisher: Wiley-Liss; 5th edition, 2005; Animal Cell Culture Methods, Methods in Cell Biology, Vol. 57, Mather, J. P. and Barnes, D. (eds.), Academic Press, 1st edition, 1998 which are all incorporated by reference herein in their entireties.

Methods for the production of antibodies are well known in the art, e.g. disclosed in PCT publication WO 97/40072 or U.S. Application. No. 2002/0182702, which are herein incorporated by reference. The processes of immunization to elicit antibody production in a mammal, the generation of hybridomas to produce monoclonal antibodies, and the purification of antibodies can be performed by described in Current Protocols in Immunology (CPI), John Wiley and Sons, Inc.; Antibodies: A Laboratory Manual, Harlow, E. and Lane, D. (eds.), Cold Spring Harbor Laboratory Press 1988; and Brown, “Clinical Use of Monoclonal Antibodies,” in Biotechnology and Pharmacy pp. 227-49, Pezzuto et al. (eds.) Chapman & Hall 1993. These references are all incorporated by reference herein in their entireties.

It should be understood that embodiments of the methods, devices and kits disclosed herein are not limited to the particular methodology, protocols, and reagents, etc., described herein and as such can vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.”

The singular terms “a,” “an,” and the include plural referents unless context clearly indicates otherwise. Similarly, the word or is intended to include and unless the context clearly indicates otherwise. It is further to be understood that all base sizes given for nucleic acids and amino acids are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the methods, devices and kits disclosed herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### The Biomarker Proteins of an Active Fibrotic Condition/Disease

The human RCN-1, reticulocalbin 1, is a 331 amino acid, calcium-binding protein located in the lumen of the ER. The protein contains six conserved regions with similarity to a high affinity Ca²⁺-binding motif, the EF-hand. High conservation of amino acid residues outside of these motifs, in comparison to mouse reticulocalbin, is consistent with a possible biochemical function besides that of calcium binding. RCN-1 is also known as RCN; RCAL; PIG20; and F1137041. The GENBANK™ Accession No. for the mRNA is NM_{—}002901.1 and the encoded protein is NP_{—}002892 or 4506455. The human RCN-1 protein has the sequence MARGGRGRRLGLALGLLLALVLAPRVLRAKPTVRKERVVRPDSELGERPPEDNQSFQYDHEAFLGKE DSKTFDQLTPDESKERLGKIVDRIDNDGDGFVTTEELKTWIKRVQKRYIFDNVAKVWKDYDRDKDDKI SWEEYKQATYGYYLGNPAEFHDSSDHHTFKKMLPRDERRFKAADLNGDLTATREEFTAFLHPEEFEH MKEIVVLETLEDIDKNGDGFVDQDEYIADMFSHEENGPEPDWVLSEREQFNEFRDLNKDGKLDKDEIR HWILPQDYDHAQAEARHLVYESDKNKDEKLTKEEILENWNMFVGSQATNYGEDLTKNHDEL (SEQ. ID. NO: 1).

PEDF is a 418 amino acid, pigment epithelium-derived factor. PEDF is non-inhibitory member of the serpin superfamily. It exhibits neurotrophic, neuroprotective and antiangiogenic properties and is widely expressed in the developing and adult nervous systems. This subgroup corresponds to clade F1 of the serpin superfamily. PEDF is also known as SERPINF1: serpin peptidase inhibitor, clade F, member 1; alpha-2 antiplasmin; proliferation-inducing protein 35; EPC-1; and PIG35. The GENBANK™ Accession No. for the mRNA is NM_{—}002615.4 and the encoded protein is NP_{—}002606 or 39725934. The human PEDF protein has the sequence MQALVLLLCIGALLGHSSCQNPASPPEEGSPDPDSTGALVEEEDPFFKVPVNKLAAAVSNFGYDLYRVR SSTSPTTNVLLSPLSVATALSALSLGAEQRTESIIHRALYYDLISSPDIHGTYKELLDTVTAPQKNLKSAS RIVFEKKLRIKSSFVAPLEKSYGTRPRVLTGNPRLDLQEINNWVQAQMKGKLARSTKEIPDEISILLLGV AHFKGQWVTKFDSRKTSLEDFYLDEERTVRVPMMSDPKAVLRYGLDSDLSCKIAQLPLTGSMSIIFFLP LKVTQNLTLIEESLTSEFIHDIDRELKTVQAVLTVPKLKLSYEGEVTKSLQEMKLQSLFDSPDFSKITGKP IKLTQVEHRAGFEWNEDGAGTTPSPGLQPAHLTFPLDYHLNQPFIFVLRDTDTGALLFIGKILDPRGP (SEQ. ID. NO: 2).

ENO1, a 434 amino acid alpha enolase 1, one of three enolase isoenzymes found in mammals. This gene that encodes alpha-enolase, a homodimeric soluble enzyme, also encodes a shorter monomeric structural lens protein, tau-crystallin. The two proteins are made from the same mRNA message. The full length protein, the isoenzyme, is found in the cytoplasm. The shorter protein is produced from an alternative translation start, is localized to the nucleus, and has been found to bind to an element in the c-myc promoter. A pseudogene has been identified that is located on the other arm of the same chromosome. Other names are NNE; PPH; MPB1; and ENO1L1. The GENBANK™ Accession No. for the mRNA is NM_{—}001428.2 and the encoded protein is NP_{—}001419 or 450371. The human ENO1 protein has the sequence MSILKIHAREIFDSRGNPTVEVDLFTSKGLFRAAVPSGASTGIYEALELRDNDKTRYMGKGVSKAVEHI NKTIAPALVSKKLNVTEQEKIDKLMIEMDGTENKSKFGANAILGVSLAVCKAGAVEKGVPLYRHIADL AGNSEVILPVPAFNVINGGSHAGNKLAMQEFMILPVGAANFREAMRIGAEVYHNLKNVIKEKYGKDAT NVGDEGGFAPNILENKEGLELLKTAIGKAGYTDKVVIGMDVAASEFFRSGKYDLDFKSPDDPSRYISPD QLADLYKSFIKDYPVVSIEDPFDQDDWGAWQKFTASAGIQVVGDDLTVTNPKRIAKAVNEKSCNCLLL KVNQIGSVTESLQACKLAQANGWGVMVSHRSGETEDTFIADLVVGLCTGQIKTGAPCRSERLAKYNQ LLRIEEELGSKAKFAGRNFRNPLAK (SEQ. ID. NO: 3).

RCN-3, reticulocalbin 3, is a 328 amino acid protein that have an EF-hand, calcium binding motif. It is a member of a diverse superfamily of calcium sensors and calcium signal modulators. Most members of this superfamily have two active canonical EF Ca²⁺ binding hands. Other names for RCN-3 include reticulocabin, reticulocalbin-like, and RLP49. The GENBANK™ Accession No. for the mRNA is NM_{—}020650.2 and the encoded protein is NP_{—}065701.2 or 28626510. The human RCN-3 protein has the sequence MMWRPSVLLLLLLLRHGAQGKPSPDAGPHGQGRVHQAAPLSDAPHDDAHGNFQYDHEAFLGREVAK EFDQLTPEESQARLGRIVDRMDRAGDGDGWVSLAELRAWIAHTQQRHIRDSVSAAWDTYDTDRDGR VGWEELRNATYGHYAPGEEFHDVEDAETYKKMLARDERRFRVADQDGDSMATREELTAFLHPEEFP HMRDIVIAETLEDLDRNKDGYVQVEEYIADLYSAEPGEEEPAWVQTERQQFRDFRDLNKDGHLDGSE VGHWVLPPAQDQPLVEANHLLHESDTDKDGRLSKAEILGNWNMFVGSQATNYGEDLTRHHDEL (SEQ. ID. NO: 4).

α-SMA, alpha smooth muscle actin is also known as actin alpha 2 (aka AAT6; ACTSA; ACTA2). α-SMA is a 377 amino acid protein encoded by the human aortic smooth muscle actin gene. It is one of six different actin isoforms which have been identified. Actins are highly conserved proteins that are involved in cell motility, structure and integrity. Alpha actins are a major constituent of any contractile apparatus in an organism. The GENBANK™ Accession No. for the mRNA is NM_{—}001613.1 and the encoded protein is NP_{—}001604.1. The human α-SMA protein has the sequence MCEEEDSTALVCDNGSGLCKAGFAGDDAPRAVFPSIVGRPRHQGVMVGMGQKDSYVGDEAQSKRGI LTLKYPIEHGIITNWVDDMEKIWHHSFYNELRVAPEEHPTLLTEAPLNPKANREKMTQIMFETFNVPAMY VAIQAVLSLYASGRTTGIVLDSGDGVTHNVPIYEGYALPHAIMRLDLAGRDLTDYLMKILTERGYSFVT TAEREIVRDIKEKLCYVALDFENEMATAASSSSLEKSYELPDGQVITIGNERFRCPETLFQPSFIGMESAG IHETTYNSIMKCDIDIRKDLYANNVLSGGTTMYPGIADRMQKEITALAPSTMKIKIIAPPERKYSVWIGG SILASLSTFQQMWISKQEYDEAGPSIVHRKCF (SEQ. ID. NO: 5).

Alpha 1 actin, skeletal muscle (ACTA1) is another member of the actin family of proteins, which are highly conserved proteins that play a role in cell motility, structure and integrity. Other names are ACTA; ASMA; CFTD; MPFD; NEM1; NEM2; NEM3; CFTD1; and CFTDM. Alpha, beta and gamma actin isoforms have been identified, with alpha actins being a major constituent of the contractile apparatus, while beta and gamma actins are involved in the regulation of cell motility. This actin is an alpha actin that is found in skeletal muscle. Mutations in this gene cause nemaline myopathy type 3, congenital myopathy with excess of thin myofilaments, congenital myopathy with cores, and congenital myopathy with fiber-type disproportion, diseases that lead to muscle fiber defects. The GENBANK™ Accession No. for the mRNA is NM_{—}001100 and the encoded protein is NP_{—}00109 or 4501881. The human ACTA1 protein has the sequence MCDEDETTALVCDNGSGLVKAGFAGDDAPRAVFPSIVGRPRHQGVMVGMGQKDSYVGDEAQSKRGI LTLKYPIEHGIITNWVDDMEKIWHHTFYNELRVAPEEHPTLLTEAPLNPKANREKMTQIMFETFNVPAMY VAIQAVLSLYASGRTTGIVLDSGDGVTHNVPIYEGYALPHAIMRLDLAGRDLTDYLMKILTERGYSFVT TAEREIVRDIKEKLCYVALDFENEMATAASSSSLEKSYELPDGQVITIGNERFRCPETLFQPSFIGMESAG IHETTYNSIMKCDIDIRKDLYANNVMSGGTTMYPGIADRMQKEITALAPSTMKIKIIAPPERKYSVWIGG SILASLSTFQQMWITKQEYDEAGPSIVHRKCF (SEQ. ID. NO: 6).

Osteonectin (ON, ONC or SPARC) is a secreted acidic and cysteine rich protein. It is a matrix-associated protein that elicits changes in cell shape, inhibits cell-cycle progression, and influences the synthesis of extracellular matrix (ECM). In the ECM, ON is a glycoprotein in the bone that binds calcium. It is secreted by osteoblasts during bone formation, initiating mineralization and promoting mineral crystal formation. ON also shows affinity for collagen in addition to bone mineral calcium. The GENBANK™ Accession No. for the mRNA is NM_{—}003118 and the encoded protein is NP_{—}003109 or 4507171. The human osteonectin protein has the sequence MRAWIFFLLCLAGRALAAPQQEALPDETEVVEETVAEVTEVSVGANPVQVEVGEFDDGAEETEEEVV AENPCQNHHCKHGKVCELDENNTPMCVCQDPTSCPAPIGEFEKVCSNDNKTFDSSCHFFATKCTLEGT KKGHKLHLDYIGPCKYIPPCLDSELTEFPLRMRDWLKNVLVTLYERDEDNNLLTEKQKLRVKKIHENE KRLEAGDHPVELLARDFEKNYNMYIFPVHWQFGQLDQHPIDGYLSHTELAPLRAPLIPMEHCTTRFFET CDLDNDKYIALDEWAGCFGIKQKDIDKDLVI (SEQ. ID. NO: 7).

α2 chain of type I collagen (COL1A2), also known as OI4, is a structural protein whose triple helix comprises two alpha1 chains and one alpha2 chain. Type I is a fibril-forming collagen found in most connective tissues and is abundant in bone, cornea, dermis and tendon. The GENBANK™ Accession No. for the mRNA is NM_{—}000089.3 and the encoded protein is NP_{—}000080 or 48762934. The human COL1A2 protein has the sequence MLSFVDTRTLLLLAVTLCLATCQSLQEETVRKGPAGDRGPRGERGPPGPPGRDGEDGPTGPPGPPGPPG PPGLGGNFAAQYDGKGVGLGPGPMGLMGPRGPPGAAGAPGPQGFQGPAGEPGEPGQTGPAGARGPA GPPGKAGEDGHPGKPGRPGERGVVGPQGARGFPGTPGLPGFKGIRGHNGLDGLKGQPGAPGVKGEPG APGENGTPGQTGARGLPGERGRVGAPGPAGARGSDGSVGPVGPAGPIGSAGPPGFPGAPGPKGEIGAV GNAGPAGPAGPRGEVGLPGLSGPVGPPGNPGANGLTGAKGAAGLPGVAGAPGLPGPRGIPGPVGAAG ATGARGLVGEPGPAGSKGESGNKGEPGSAGPQGPPGPSGEEGKRGPNGEAGSAGPPGPPGLRGSPGSR GLPGADGRAGVMGPPGSRGASGPAGVRGPNGDAGRPGEPGLMGPRGLPGSPGNIGPAGKEGPVGLPGI DGRPGPIGPAGARGEPGNIGFPGPKGPTGDPGKNGDKGHAGLAGARGAPGPDGNNGAQGPPGPQGVQ GGKGEQGPPGPPGFQGLPGPSGPAGEVGKPGERGLHGEFGLPGPAGPRGERGPPGESGAAGPTGPIGSR GPSGPPGPDGNKGEPGVVGAVGTAGPSGPSGLPGERGAAGIPGGKGEKGEPGLRGEIGNPGRDGARGA PGAVGAPGPAGATGDRGEAGAAGPAGPAGPRGSPGERGEVGPAGPNGFAGPAGAAGQPGAKGERGA KGPKGENGVVGPTGPVGAAGPAGPNGPPGPAGSRGDGGPPGMTGFPGAAGRTGPPGPSGISGPPGPPG PAGKEGLRGPRGDQGPVGRTGEVGAVGPPGFAGEKGPSGEAGTAGPPGTPGPQGLLGAPGILGLPGSR GERGLPGVAGAVGEPGPLGIAGPPGARGPPGAVGSPGVNGAPGEAGRDGNPGNDGPPGRDGQPGHKG ERGYPGNIGPVGAAGAPGPHGPVGPAGKHGNRGETGPSGPVGPAGAVGPRGPSGPQGIRGDKGEPGEK GPRGLPGLKGHNGLQGLPGIAGHHGDQGAPGSVGPAGPRGPAGPSGPAGKDGRTGHPGTVGPAGIRGP QGHQGPAGPPGPPGPPGPPGVSGGGYDFGYDGDFYRADQPRSAPSLRPKDYEVDATLKSLNNQIETLL TPEGSRKNPARTCRDLRLSHPEWSSGYYWIDPNQGCTMDAIKVYCDFSTGETCIRAQPENIPAKNWYR SSKDKKHVWLGETINAGSQFEYNVEGVTSKEMATQLAFMRLLANYASQNITYHCKNSIAYMDEETGN LKKAVILQGSNDVELVAEGNSRFTYTVLVDGCSKKTNEWGKTIIEYKTNKPSRLPFLDIAPLDIGGADQ EFFVDIGPVCFK (SEQ. ID. NO: 8).

Tropomyosin 4 (TMP4) is a member of the tropomyosin family of actin-binding proteins involved in the contractile system of striated and smooth muscles and the cytoskeleton of non-muscle cells. Tropomyosins are dimers of coiled-coil proteins that polymerize end-to-end along the major groove in most actin filaments. They provide stability to the filaments and regulate access of other actin-binding proteins. In muscle cells, they regulate muscle contraction by controlling the binding of myosin heads to the actin filament. Multiple transcript variants encoding different isoforms have been found for this gene, isoform 1 and 2. Other names are tropomyosin alpha-4 chain and TM30p1. The GENBANK™ Accession No. for the mRNA is NM_{—}001145160 and NM_{—}003290.2 and the encoded protein are NP_{—}001138632.1, NP_{—}003281.1, or 4507651 depending on the isoform selected. The human longer isoform 1 TMP4 protein has the sequence MEAIKKKMQMLKLDKENAIDRAEQAEADKKAAEDKCKQVEEELTHLQKKLKGTEDELDKYSEDLKD AQEKLELTEKKASDAEGDVAALNRRIQLVEEELDRAQERLATALQKLEEAEKAADESERGMKVIENRA MKDEEKMEIQEMQLKEAKHIAEEADRKYEEVARKLVILEGELERAEERAEVSELKCGDLEEELKNVTN NLKSLEAASEKYSEKEDKYEEEIKLLSDKLKEAETRAEFAERTVAKLEKTIDDLEEKLAQAKEENVGLH QTLDQTLNELNCI (SEQ. ID. NO: 9) and the human shorter isoform 2 of the TMP4 protein has the sequence MAGLNSLEAVKRKIQALQQQADEAEDRAQGLQRELDGERERREKAEGDVAALNRRIQLVEEELDRAQ ERLATALQKLEEAEKAADESERGMKVIENRAMKDEEKMEIQEMQLKEAKHIAEEADRKYEEVARKLV ILEGELERAEERAEVSELKCGDLEEELKNVTNNLKSLEAASEKYSEKEDKYEEEIKLLSDKLKEAETRAE FAERTVAKLEKTIDDLEEKLAQAKEENVGLHQTLDQTLNELNCI″ (SEQ. ID. NO: 10).

Calreticulin (CALR) is a multifunctional precursor protein that acts as a major Ca²⁺-binding (storage) protein in the lumen of the endoplasmic reticulum. It is also found in the nucleus, indicating that it may have a role in transcription regulation. CALR binds to the DNA-binding domain of the superfamily of nuclear receptors, e.g. the steroid hormone receptors (e.g. glucocorticoid, androgen, and retinoic acid). CALR play an important role in modulating the regulation of gene transcription by nuclear hormone receptors. Other names of CALR are RO; CRT; SSA; cClqR; and FLJ26680. The GENBANK™ Accession No. for the mRNA is 1.NM_{—}004343.3 and the encoded protein is NP_{—}004334.1. The human CALR protein is MLLSVPLLLGLLGLAVAEPAVYFKEQFLDGDGWTSRWIESKHKSDFGKFVLSSGKFYGDEEKDKGLQT SQDARFYALSASFEPFSNKGQTLVVQFTVKHEQNIDCGGGYVKLFPNSLDQTDMHGDSEYNIMFGPDI CGPGTKKVHVIFNYKGKNVLINKDIRCKDDEFTHLYTLIVRPDNTYEVKIDNSQVESGSLEDDWDFLPP KKIKDPDASKPEDWDERAKIDDPTDSKPEDWDKPEHIPDPDAKKPEDWDEEMDGEWEPPVIQNPEYK GEWKPRQIDNPDYKGTWIHPEIDNPEYSPDPSIYAYDNFGVLGLDLWQVKSGTIFDNFLITNDEAYAEE FGNETWGVTKAAEKQMKDKQDEEQRLKEEEEDKKRKEEEEAEDKEDDEDKDEDEEDEEDKEEDEEE DVPGQAKDEL (SEQ. ID. NO: 11).

Extracellular and Intracellular Sources for Biological Samples and their Preparation

Collections of biological samples from extracellular source such as whole blood, serum, plasma, urine, saliva, cerebrospinal fluid and bronco alveolar lavage can be performed by any methods known in the art.

For example, the patient's blood can be drawn by trained medical personnel directly into anti-coagulants such as citrate and EDTA. The whole blood can be separated into the plasma portion and the cell portion by refrigerated centrifugation at 3500×G for 2 minutes. After centrifugation, the supernatant is the plasma. This can be stored at −20° or −80° C. before analysis of the biomarkers is performed.

Alternately, the serum can be collected from the whole blood. Collect the blood in a hard plastic or glass tube; blood will not clot in soft plastic. Draw 15 mL of whole blood for 6 mL of serum. The whole blood is allowed to stand at room temperature for 30 minutes to 2 hours until a clot has formed. Carefully separate clot from the sides of the container using a glass rod or wooden applicator stick and leave overnight at 4° C. After which, decant serum, centrifuge, and/or using a Pasteur pipette, remove serum into a clean tube. Clarify the serum by centrifugation at 2000-3000 rpm for 10 minutes. The serum is stored at −20° or −80° C. before analysis of the biomarkers is performed. Detailed description of obtaining serum using collection tubes can be found in U.S. Pat. No. 3,837,376 and is hereby incorporated by reference in it entirety. Blood collection tubes can also be purchased from BD Diagnostic Systems, Greiner Bio-One, and Kendall Company.

Bronco alveolar lavage (BAL) is a procedure that involves wedging the tip of a bronchoscope into a bronchus (subsegmental), instilling a known volume of saline solution into the distal airway, then aspirating up this volume of saline and sending it to the laboratory for analysis. BAL is typically performed to diagnose lung disease. In particular, BAL is commonly used to diagnose infections in people with immune system problems, pneumonia in people on ventilators, some types of lung cancer, and scarring of the lung (interstitial lung disease). BAL is the most common manner to sample the components of the epithelial lining fluid (ELF) and to determine the protein composition of the pulmonary airways, and it is often used in immunological research as a means of sampling cells or pathogen levels in the lung. A skilled clinician or technician should be able to obtain samples via BAL in an individual.

Collections of intracellular source samples, such as skin or lung tissue, can be performed by any methods well known to those skilled in the art.

Tissue biopsies are some of the intracellular sources for the methods described herein. Such tissues can be culture ex vivo as described in Example 1. While not wishing to be bound by theory, it is presumed that the fibroblast in the excised tissue will continue to remain viable in culture, and also continue to produce the proteins and materials involved in the fibrotic process. It is also envisioned that some of these proteins and materials are exported extracellularly and are found in the culture media. Therefore, the spent media of an ex vivo tissue culture can be prepared for the measurement of the level of the biomarker proteins by an antibody-based or MS analyses described herein. In other embodiments, such tissues are prepared for an antibody-based or MS analyses which measure the level of the biomarker proteins. Such methods are well known in the art. Examples of preparation protocols are below. It should not be construed that the practice of the methods, device and kit are limited to these protocols only.

### Tissue Lysate Preparation Protocol

Weigh tissue sample in a 50 ml tube. While keeping sample on ice, wash with cold 1×PBS and aspirate off PBS. Repeat until wash buffer appears clear. Add sufficient volume of cold lysis buffer to cover sample (about 3 times the weight of sample in volume; i.e. 500 mg tissue sample will receive 1.5 ml lysis buffer). Grind/homogenize tissue in tube and incubate on ice for at least one hour. Transfer mixture to microcentrifuge tubes and spin at 14,000 rpm for 30 min at 4° C. Poke through lipid layer and remove supernatant (this is the lysate). Throw away cellular debris and lipids. If necessary, respin supernatant at 14,000 rpm and repeat the previous step to obtain clean lysate free of lipid and debris. Measure the protein concentration using the Bradford protein assay. Store at −80° C. until ready to work up. Avoid freeze/thawing to protein as much as possible.

5× Lysis buffer composition (stored in 4° C.) comprises the following: 50 mM Tris pH 7.5, 650 mM NaCl, 5% Triton-X100, 50 mM NaF, 50 mM NaPi pH 7.5 (sodium phosphate), 50 mM NaPPi pH 7.5 (sodium pyrophosphate). These are added right before use: 0.02 mg/mL RNase, 0.2 mg/ml Dnase, 1 mM PMSF, 1× protease inhibitor cocktail (PIC). 100×PIC: 1.6 mg/ml Benzamidine HCl, 1.0 mg/mL phenanthroline, 1.0 mg/ml aprotonin, 1.0 mg/ml leupeptin, 1.0 mg/ml pepstatin A, dissolve in 100% ethanol and stored at −20° C.

Alternatively, the tissue sample can be immediately homogenized in TRIZOL or TRI REGENT®. TRI REAGENT® is a complete and ready-to-use reagent for the isolation of total RNA or the simultaneous isolation of RNA, DNA and proteins from samples of human, animal, plant, yeast, bacterial and viral origin. Alternatively, the FASTPURE™ RNA Kit from Takara Bio World Wide Website) can be used. Detailed protocol can be found in U.S. Pat. Nos. 4,843,155 and 5,346,994, at the manufacturer's World Wide Website.

RNA Isolation and cDNA Synthesis

Total RNA isolated from the excised tissue by the Trizol method can be quantitated using a Smart Spec 3000 (Bio-Rad, Hercules, Calif.). The mRNA transcripts are then transcribed into cDNAs using the Novagen directional synthesis kit and oligo dT primers. For ratiometric RT-PCR and quantitative RT-PCR, cDNAs, 4 μg of total RNA can used for the ISCRIPT™ cDNA synthesis kit with oligo dT primers.

### Measuring the Level of Biomarker Proteins

In embodiments, the level of a fibrotic biomarker protein, such as those disclosed in Tables 4 and 5, and in particular, the following biomarkers: ENO1, RCN-3, α-SMA, RCN-1 and PEDF, is measured to obtain a diagnosis of whether a human patient has SSc, NSF or a chronic fibrotic condition. A biomarker protein level can be measured using any assay known to those of ordinary skilled in the art, including, but not limited to, Enzyme-Linked Immunosorbent Assay (ELISA), immunoprecipitation assays, radioimmunoassay, mass spectrometry, Western Blotting, and other modified immunoassays e.g. dipsticks using conventional technology.

In one embodiment, the levels of the biomarker proteins described herein can be measured by contacting biological samples containing the biomarker proteins with an antibody-based binding moiety or a protein-binding agent that specifically binds to the respective biomarkers. The samples can be, but are not limited to, whole blood, plasma, urine, saliva, cerebrospinal fluid, serum, spent media of primary fibroblast culture of excised tissues from an individual, and tissue lysate from excised tissues from an individual. The samples can be deposited on a solid support such as a nitrocellulose or polyvinylidene fluoride (PVDF) membrane. Such solid supports are well known in the art, particularly in Western Blots and dot blots. In some embodiments, the molecules in the samples deposited on a solid support are electrophoretically separated from each other by size and electric charge. Incubation of samples with antibody-based binding moiety or protein-binding agent that specifically binds to the respective biomarkers results in the formation of antibody-biomarker proteins complexes or protein-binding agent-biomarker proteins complexes, which is then detected by a variety of methods known in the art.

For purposes of comparison, the level of a biomarker protein in a biological sample from the patient should be measured in the same manner as the reference amount is measured. For example, the levels of biomarker proteins can be represented in arbitrary units dependent upon the assay used to measure the levels of biomarker proteins, e.g., the intensity of the signal from the detectable label can correspond to the amount of biomarker proteins present (e.g. as determined by eye, densitometry, an ELISA plate reader, a luminometer, or a scintillation counter).

The level of a biomarker protein present in a biological sample can be measured using any protein-binding agent. In some embodiments, a protein-binding agent is a ligand that specifically binds to a biomarker protein, and can be, for example, a synthetic peptide, chemical, small molecule, or antibody or antibody fragment or variants thereof (otherwise also known as an antibody-based binding moiety). In some embodiments, a protein-binding agent is a ligand or antibody or antibody fragment, and in some embodiments, a protein-binding agent is preferably detectably labeled.

In one embodiment, immunoassays using antibodies or antibody-based binding moieties are used to measure the levels of biomarker proteins. As used herein, the term “antibody” is intended to include immunoglobulin molecules and immunologically active determinants of immunoglobulin molecules, e.g., molecules that contain an antigen binding site which specifically binds (immunoreacts with) to the biomarker to be measured. The term “antibody” is intended to include whole antibodies, e.g., of any isotype (IgG, IgA, IgM, IgE, etc), and includes fragments thereof which are also specifically reactive with the biomarker proteins to be measured, e.g. ENO1, RCN-3, α-SMA, RCN-1 and PEDF. Antibodies can be fragmented using conventional techniques. Thus, the term “antibody” includes segments of proteolytically-cleaved or recombinantly-prepared portions of an antibody molecule that are capable of selectively reacting with a certain protein. Non limiting examples of such proteolytic and/or recombinant fragments include Fab, F(ab′)₂, Fab′, Fv, dAbs and single chain antibodies (scFv) containing a VL and VH domain joined by a peptide linker. The scFv's can be covalently or non-covalently linked to form antibodies having two or more binding sites. Thus, “antibody” includes polyclonal, monoclonal, or other purified preparations of antibodies and recombinant antibodies. The term “antibody” is further intended to include humanized antibodies, bispecific antibodies, and chimeric molecules having at least one antigen binding determinant derived from an antibody molecule. In one embodiment, the antibody is detectably labeled.

Antibodies to the biomarker proteins disclosed herein can be generated using any methods known to those skilled in the art. Alternatively, commercially available antibodies can be used. Antibodies to ENO1, RCN-3, α-SMA, RCN-1, ACTA1 and PEDF are commercially available.

As used herein “detectably labeled”, includes antibodies that are labeled by a measurable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, HIS, or biotin.

In the embodiments of the diagnostic and prognostic methods described herein that use an antibody for the measurement of biomarker proteins levels, the level of biomarker proteins biological present in a biological sample correlates to the intensity of the signal emitted from the detectably labeled antibody.

In one embodiment, the protein-binding agent in the protein-binding agent-biomarker complex or antibody-biomarker proteins complex is detectably labeled by linking the antibody to an enzyme. The enzyme, in turn, when exposed to its substrate, will react with the substrate in such a manner as to produce a chemical moiety which can be detected, for example, by spectrophotometric, fluorometric or by visual means. Enzymes which can be used to detectably label the antibodies that specifically binds the biomarker proteins are well known in the art, and they include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection of biomarker proteins can also be accomplished using any of a variety of other immunoassays. For example, by radioactively labeling a protein-binding agent that specifically binds to the respective biomarkers, it is possible to detect the protein-binding agent through the use of radioimmune assays. The radioactive isotope can be detected by such means as the use of a gamma counter or a scintillation counter or by audioradiography. Isotopes which are particularly useful for the purpose of the present invention are ³H, ¹³¹I, ³⁵S, ¹⁴C, and preferably ¹²⁵I.

In addition, it is also possible to label a protein-binding agent that specifically binds to the respective biomarkers with a fluorescent compound. When the fluorescently labeled protein-binding agent is exposed to light of the proper wavelength, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine.

A protein-binding agent can also be detectably labeled using fluorescence emitting metals such as ¹⁵²Eu, or others of the lanthanide series. These metals can be attached to the protein-binding agent using such metal chelating groups as diethylenetriaminepentaacetic acid (DTPA) or ethylenediaminetetraacetic acid (EDTA).

A protein-binding agent also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

In one embodiment, the biomarker proteins are determined and quantitated by Western blots.

As an exemplary, to measure the amount of protein expression of RCN-1, ENO1, or PEDF, 50 μg of protein from the samples described herein are resolved in 10% (w/v) SDS-polyacrylamide gels on a Minigel apparatus (BIO-RAD®) and transferred to a PVDF membrane (Immobilon™-P, Millipore) using a semidry transfer (BIO-RAD®). Known standard amounts of RCN-1, ENO1, and PEDF are also included in the gel as positive controls and standards for densitometry or fluorescence quantification. Commercially available proteins such as recombinant protein ENO1 (P02) from Cedarlanelabs, recombinant PEDF protein (catalog No. P3140-02) from U.S. Biologicals, and human recombinant RCN-1 (catalog No. 157H00005954-P01-0010) from Tebu-bio can be used as ENO1, PEDF and RCN-1 positive controls and standards respectively. The membrane is blocked for 1 h with 3% (w/v) BSA in TBS (273.8 mM NaCl, 5.4 mM KCl, and 38.1 mM Tris) with 1% (v/v) Tween 20, and then is incubated with the primary antibody at the appropriate dilution, rabbit anti-reticulocalbin 1:3,000 (prepared commercially by Bethyl Laboratories, Montgomery TX and targets the EF-hand domain of the protein), rabbit anti-ENO1 (human) (GenWay Biotech Inc. Catalog Number: 18-003-42514) diluted to 1.25 μg/ml in 5% skim milk/PBS buffer, mouse anti-PEDF (1C4) 1:2500 (Santa Cruz Biotechnologies, Santa Cruz, Calif.; sc-53921) rabbit anti-actin 1:5000 (Sigma), and rabbit anti-131-integrin 1:2,000 (Santa Cruz, Santa Cruz, Calif.), in 3% (w/v) BSA in TBS. Anti-131-integrin is used as a loading control for membrane preparation. Anti-primary antibody exposure was carried out at 4° C. for 18 hr and membranes then washed 3 times with TBS-T (273.8 mM NaCl, 5.4 mM KCl, 38.1 mM Tris, and 0.1% (v/v) Tween 20). For RCN-1 and ENO1 detection, the membrane is probed with goat anti-rabbit IgG HRP-linked 1:10,000 (Cell Signaling, Danvers, M A) for 1 h at room temperature and washed four times with TBS-T. For PEDF detection, the membrane is probed with goat anti-mouse IgG HRP-linked 1:30,000 (Cell Signaling, Danvers, M A). For β-actin and 131-integrin immunodetection, the membranes are probed with goat anti-rabbit IgG HRP-linked 1:20,000 (Cell Signaling), incubated for 1 hr at room temperature, and washed four times with TBST. The immunoblots are visualized by chemiluminescence with LUMIGLO® (Cell Signaling). Membranes are treated with stripping buffer (100 mM β-mercaptoethanol, 2% (w/v) SDS, 62.5 mM Tris-HCl) for 45 min at 70° C., washed twice with TBST for 15 min, then were re-blocked and probed. The biomarkers are quantified by densitometry that is well known in the art. In some embodiments, the Western Blots are performed three times for each sample.

In some embodiments, the levels of biomarkers described herein are be detected by immunoassays, such as enzyme linked immunoabsorbent assay (ELISA), radioimmunoassay (RIA), immunoradiometric assay (IRMA), Western blotting, diffusion immunoassay (DIA), fluoroimmunoassay (FIA), chemiluminescent immunoassay (CLIA), counting immunoassay (CIA), lateral flow tests or immunoassay (LFIA), also known as lateral flow immunochromatographic assays, magnetic immunoassay (MIA), and immunohistochemistry, which are well known in the art and are described in more detail below. Immunoassays such as ELISA or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos. 6,329,209 and 6,365,418, which are herein incorporated by reference in their entirety.

### Immunoassays

An immunoassay is a biochemical test that measures the concentration of a substance in a biological sample, typically serum, plasma, urine, saliva etc, using the reaction of an antibody or antibodies to its antigen. The assay takes advantage of the specific binding of an antibody to its antigen. Monoclonal antibodies are often used as they only usually bind to one site of a particular molecule/antigen, and therefore provide a more specific and accurate test, which is less easily confused by the presence of other molecules. The antibodies picked must have a high affinity for the antigen (if there is antigen available, a very high proportion of it must bind to the antibody).

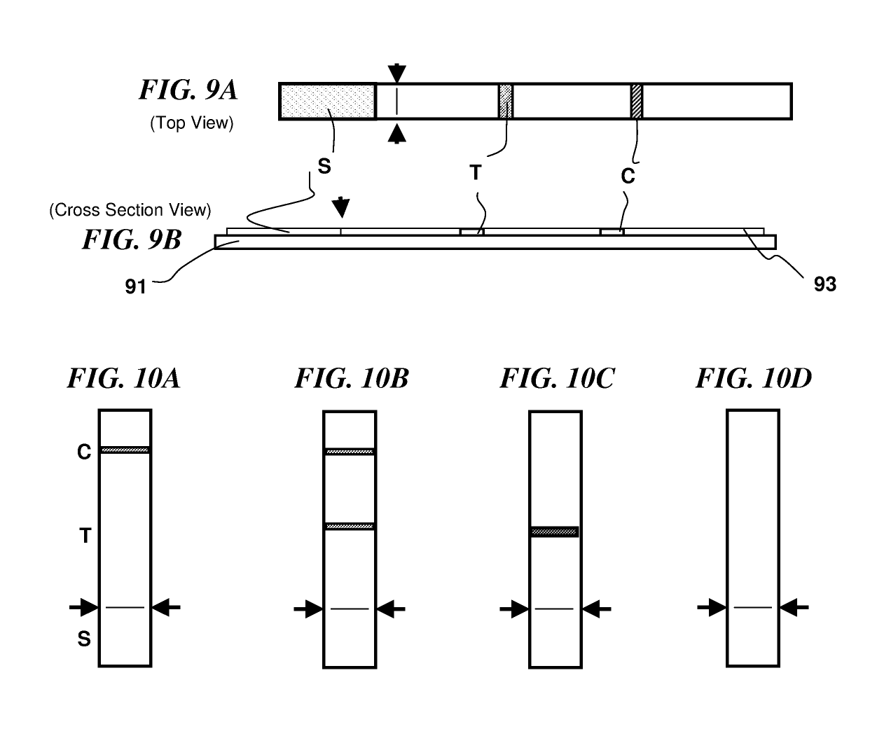

For numerical results, the response of the biological sample being measured must be compared to standards of a known concentration. This is usually done through the plotting of a standard curve on a graph, the position of the curve at response of the unknown is then examined, and so the quantity of the unknown found. Alternatively, a defined amount of antibody is used in the assay where the defined amount of antibody binds completely to a fixed amount of antigen. This fixed amount of antigen is the reference level of biomarker in a sample. Thus, this defined amount of antibody is used to indicate whether the amount of antigen in the biological sample is at least at, below or above the reference level of biomarker (See FIGS. 9-10).

Measuring the quantity of antigen in the biological sample can be achieved by a variety of methods known to one skilled in the art. One of the most common is to label either the antigen or the antibody. The label can consist of an enzyme (see enzyme immunoassay (EIA)), colloidal gold (lateral flow assays), radioisotopes such as I-¹²⁵ Radioimmunoassay (RIA), magnetic labels (magnetic immunoassay—MIA) or fluorescence. Other techniques include agglutination, nephelometry, turbidimetry and Western Blot.

In one embodiment, the immunoassay is a competitive immunoassay. In another embodiment, the immunoassay is a noncompetitive immunoassay.

Immunoassays can be divided into those that involve labeled reagents and those which involve non-labeled reagents. Those which involve labeled reagents are divided into homogenous and heterogeneous (which require an extra step to remove unbound antibody or antigen from the site, usually using a solid phase reagent) immunoassays. Heterogeneous immunoassays can be competitive or non-competitive.

In a competitive immunoassay, the antigen in the unknown sample competes with labeled antigen to bind with antibodies. The amount of labeled antigen bound to the antibody is then measured. In this method, the response will be inversely proportional to the concentration of antigen in the unknown. This is because the greater the response, the less antigen in the unknown was available to compete with the labeled antigen.

In noncompetitive immunoassays, also referred to as the “sandwich assay,” antigen in the unknown, e.g. serum sample, is bound to a first antibody site, then second antibody that is labeled is bound to the antigen, forming a sandwich. The amount of labeled antibody on the site is then measured. Unlike the competitive method, the results of the noncompetitive method will be directly proportional to the concentration of the antigen. This is because labeled antibody will not bind if the antigen is not present in the unknown sample, e.g serum sample.

The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in Methods in Immunodiagnosis, 2nd Edition, Rose and Bigazzi, (eds.), John Wiley & Sons, 1980; Campbell et al., Methods and Immunology, W. A. Benjamin, Inc., 1964; and Oellerich, M., J. Clin. Chem. Clin. Biochem., 22:895-904, 1984.

For the methods described herein, in the ELISA a known amount of a first anti-biomarker antibody is affixed to a solid surface, and then the biological sample, e.g. serum, containing the biomarker of interest is washed over the surface so that the antigen biomarker can bind to the immobilized antibodies (a first antibody). The surface is washed to remove any unbound biomarker and also any non-biomarker proteins present in the serum sample. A detection antibody (a second antibody) is applied to the surface. This detection antibody can be linked to an enzyme, and in the final step a substance is added that the enzyme can convert to some detectable signal. For example, in the case of fluorescence ELISA, when light is shone upon the sample, any antigen/antibody complexes will fluoresce so that the amount of antibodies in the sample can be measured.

The following is a general standard protocol for setting up and performing an indirect enzyme-linked immunosorbent assay. Using 96-well microtiter plates (Falcon Pro-Bindassay plate 3915; Becton Dickinson, Paramus, N.J.), test wells are coated with anti-biomarker antibody by incubation with 100 μl of purified anti-ENO1 antibody (3 μg/ml in PBS) per well overnight at room temperature, with PBS substituted for the antibody in control wells. After the plates have been washed three times with PBS-Tween, 250 μl of 2% BSA in PBS is added to each well, and the plates are incubated for 1 h at room temperature. The plates are washed three times with PBS-Tween and incubated for 1 h at room temperature with test serum sample and control serum sample from healthy individuals diluted 1:100 in PBS-Tween-BSA; each serum sample is tested in triplicate in anti-ENO1 antibody-coated wells as well as in PBS control wells. The plate is then assayed (with appropriate controls) for the presence and/or the level of ENO1 by incubation for 1 h at room temperature with 100 μl of goat anti-ENO1 IgG conjugated with horseradish peroxidase (BIO-RAD®, Richmond, Calif.) per well diluted 1:2,000 in PBS-Tween-BSA. After three washes in PBS-Tween, the substrate solution (o-phenylenediamine dihydrochloride; SIGMA) is added to each well. The plates are then incubated for 30 min at room temperature in darkness, and the reaction is terminated by the addition of 2N sulfuric acid. The optical density values at 490 nm (OD₄₉₀) are measured in a micro plate ELISA reader. For each serum sample, mean OD₄₉₀ readings are calculated for the test wells and for the antigen control wells, the latter being subtracted from the former to obtain the net ELISA value.

Performing an ELISA involves at least one antibody with specificity for a particular biomarker. A known amount of anti-biomarker antibody is immobilized on a solid support (usually a polystyrene micro titer plate) either non-specifically (via adsorption to the surface) or specifically (via capture by another antibody specific to the anti-biomarker antibody, in a “sandwich” ELISA). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In a “sandwich ELISA”, an antibody (e.g. anti-ENO1 etc.) is linked to a solid phase (i.e. a microtiter plate) and exposed to a biological sample containing antigen (e.g. ENO1). The solid phase is then washed to remove unbound antigen. A labeled antibody (e.g. enzyme linked) is then bound to the bound-antigen (if present) forming an antibody-antigen-antibody sandwich. Non-limiting examples of enzymes that can be linked to the antibody are alkaline phosphatase, horseradish peroxidase, luciferase, urease, and β-galactosidase. The enzyme-linked antibody reacts with a substrate to generate a colored reaction product that can be measured.

In one embodiment, the levels of biomarkers described herein are measured with a commercially available ELISA Kit such as CHEMIKINE™ PEDF Sandwich ELISA Kit (Millipore, Catalog No. CYT420), Human α-Smooth muscle actin, α-SMA ELISA Kit (USCN Life Co. Inc., Catalog No. E134) and ENO1 by Human α-enolase ELISA kit (Uscnlife, Catalog No. E1449h). ELISA has a sensitivity of <0.05 ng/ml for PEDF and <39 pg/mL for ENO1. One skilled in the art should be able to adapt commercially available ELISA kits for use with the appropriate anti-ENO1, anti-RCN-1 or anti-PEDF antibodies suitable for ELISA. In some embodiment, any antibodies specific to any of the biomarkers listed in Tables 4 and 5 can used. For example, blood samples are collected in tubes containing EDTA, and the plasma is prepared by centrifugation (3000 rpm, 20 min) and stored at −80° C. before use. Before the assay, the samples are thawed on ice, and urea is added to the samples to a final concentration of 8 M. After incubation on ice for 1 hour, the samples are diluted in assay diluent, then immediately applied to the assay plates and measured according to the manufacturer's protocol. Recommended standard curve concentrations used for the ENO1 ELISA's are 10,000 pg/ml, 5,000 pg/ml, 2,500 pg/ml, 1,250 pg/ml, 625 pg/ml, 312.5 pg/ml, 156 pg/ml.

In a “competitive ELISA”, a labeled antibody is incubated with a sample containing antigen (e.g. RCN-1). Another anti-biomarker antibody (non labeled) is coated on the solid phase of multi-wells. A sample with a biomarker of interest and horseradish peroxidase labeled anti-biomarker antibody (secondary detection conjugated antibody) are added to the coated wells (e.g. of a microtiter plate) to form competitive combination. After incubation, if the biomarker level in the serum sample is high, a complex of biomarker/anti-biomarker antibody/anti-biomarker antibody labeled with HRP will form. Washing the wells will remove the complex. Incubation with TMB (3,3′,5,5′-tetramethylbenzidene) will result in color development substrate for the localization of horseradish peroxidase-conjugated antibodies in the wells. There will be no color change or little color change. If the biomarker level in the sample is low, there will be much color change. Such a competitive ELSA test is specific, sensitive, reproducible and easy to operate.

In an “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g. luciferase, alkaline phosphatase, horseradish peroxidase, or β-galactosidase), or chemical methods (e.g. DAB/Substrate chromagen). The sample is then analyzed microscopically, most preferably by light microscopy of a sample stained with a stain that is detected in the visible spectrum, using any of a variety of such staining methods and reagents known to those skilled in the art.

Alternatively, “radioimmunoassays” can be employed. A radioimmunoassay is a technique for detecting and measuring the concentration of an antigen using a labeled (e.g. radioactively or fluorescently labeled) form of the antigen. Examples of radioactive labels for antigens include ³H, ¹⁴C, and ¹²⁵I. The concentration of an antigen (e.g. PEDF) in a biological sample is measured by having the antigen (e.g. PEDF) in the biological sample compete with the labeled (e.g. radioactively) antigen (e.g. labeled PEDF) for binding of an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed Staphylococcus aureus. Yet another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “immunoradiometric assay” (IRMA) is an immunoassay in which the antibody reagent is radioactively labeled. An IRMA requires the production of a multivalent antigen conjugate by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

In one embodiment, the levels of biomarker proteins are measured by contacting a sample with a first antibody that specifically binds to a biomarker protein to be measured under conditions permitting formation of a complex between the antibody and the biomarker proteins (e.g. ENO1, RCN-3, α-SMA, RCN-1 and PEDF). The amount of complex formed is then measured as a measure of the level of the biomarker protein, and the amount of complex formed is compared to the amount of complex formed between the first antibody and a predetermined reference amount of the biomarker protein. This predetermined reference level amount of the biomarker protein is the amount found in a population of healthy individuals. A level above the reference amount of a biomarker protein indicates that the human has an active fibrotic condition.

In one embodiment, the first antibody is detectably labeled. Detectably labeling the first antibody is appropriate for use, for example, in standard ELISA assays where biomarker protein is absorbed to an ELISA plate, or in Western Blot analysis, or certain LFIA dipstick analyses.

In one embodiment, the first antibody is immobilized on a solid support, for example, when using a “Sandwich ELISA” or a dipstick analysis, then the amount of complex formed can measured by detecting binding of a second antibody that specifically binds to the biomarker protein (e.g. ENO1, RCN-3, α-SMA, RCN-1 and PEDF) under conditions permitting formation of a complex between the second antibody and the biomarker protein, wherein the second antibody does not substantially cross-react with the first antibody, and wherein the second antibody is detectably labeled.

Any solid support can be used, including but is not limited to, nitrocellulose, solid organic polymers, such as polystyrene, or laminated dipsticks such as described in U.S. Pat. Nos. 5,550,375 and 5,656,448, which is specifically incorporated herein by reference in their entirety.

In one embodiment, the levels of two biomarker proteins defining a first and a second biomarker protein, are measured using at least two antibodies specific to each biomarker protein to be measured. Each antibody specifically reacts either the first biomarker protein or the second biomarker protein to be measured while not substantially cross-reacting with the other biomarker proteins to be measured.

In one embodiment, the levels of three biomarker proteins defining a first biomarker protein, a second biomarker protein, and a third biomarker protein, are measured using at least three antibodies specific to each biomarker protein to be measured, wherein each antibody specifically reacts either the first biomarker protein, the second biomarker protein, or the third biomarker protein to be measured while not substantially cross-reacting with the other biomarker proteins to be measured.

In one embodiment, the levels of four biomarker proteins defining a first, a second, a third and a fourth biomarker protein, are measured using at least four antibodies specific to each biomarker protein to be measured, wherein each antibody specifically reacts either the first biomarker protein, the second biomarker protein, the third biomarker protein, or the fourth biomarker protein to be measured while not substantially cross-reacting with the other biomarker proteins to be measured.

In one embodiment, the biomarker proteins are selected from the group consisting of ENO1, RCN-3, α-SMA, RCN-1 and PEDF. In other embodiments, additional biomarkers are selected from Tables 4 and 5 and are used in combination with ENO1, RCN-3, α-SMA, RCN-1 and PEDF.

In one embodiment, the levels of biomarker proteins in a sample are detected by a lateral flow immunoassay test (LFIA), also known as the immunochromatographic assay, or strip test. LFIAs are a simple device intended to detect the presence (or absence) of a target antigen in a fluid sample. There are currently many LFIA tests are used for medical diagnostics either for home testing, point of care testing (POCT), or laboratory use. A LFIA test is a form of immunoassay in which the test sample flows along a solid substrate via capillary action. After the sample is applied to the test it encounters a coloured reagent which mixes with the sample and transits the substrate encountering lines or zones which have been pretreated with an antibody or antigen. Depending upon the antigens present in the sample the coloured reagent can become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as serum, blood, water samples etc. Strip tests are also known as dip stick test, the name bearing from the literal action of “dipping” the test strip into a fluid sample to be tested. LFIA strip test are easy to use, require minimum training and can easily be included as components of point-of-care test (POCT) diagnostics to be use on site in the field.

LFIA tests can be operated as either competitive or sandwich assays. Sandwich LFIAs are similar to sandwich ELISA. The sample first encounters coloured particles which are labeled with antibodies raised to the target antigen. The test line will also contain antibodies to the same target, although it may bind to a different epitope on the antigen. The test line will show as a coloured band in positive samples. Examples 3 and 4 illustrate sandwich LFIAs in the test strip format. Competitive LFIAs are similar to competitive ELISA. The sample first encounters coloured particles which are labeled with the target antigen or an analogue. The test line contains antibodies to the target/its analogue. Unlabelled antigen in the sample will block the binding sites on the antibodies preventing uptake of the coloured particles. The test line will show as a coloured band in negative samples.

A typical test strip consists of the following components: (1) sample application area comprising an absorbent pad (i.e. the matrix or material) onto which the test sample is applied; (2) conjugate or reagent pad—this contains antibodies specific to the target antigen (i.e. the biomarker protein) conjugated to colored particles (usually colloidal gold particles, or latex microspheres); test results area comprising a reaction membrane—typically a hydrophobic nitrocellulose or cellulose acetate membrane onto which anti-antigen antibodies are immobilized in a line across the membrane as a capture zone or test line (a control zone may also be present, containing antibodies specific for the conjugate antibodies); and (4) optional wick or waste reservoir—a further absorbent pad designed to draw the sample across the reaction membrane by capillary action and collect it. The components of the strip are usually fixed to an inert backing material and may be presented in a simple dipstick format or within a plastic casing with a sample port and reaction window showing the capture and control zones. While not strictly necessary, most tests will incorporate a second line which contains an antibody that picks up free latex/gold in order to confirm the test has operated correctly. FIGS. 9-17 show the various components and embodiments of several test strips.

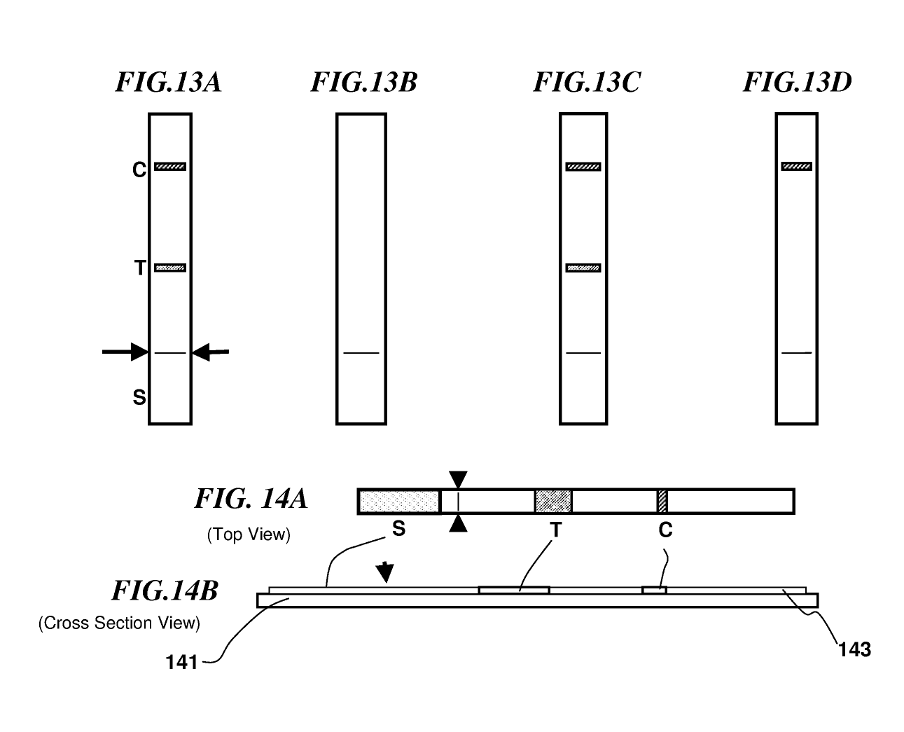

In some embodiments, the lateral flow immunoassay is a double antibody sandwich assay, a competitive assay, a quantitative assay or variations thereof. FIGS. 13, 14, 15, Example 3 and Example 4 exemplify double antibody sandwich LFIAs in a test strip format.

There are a number of variations on lateral flow technology. It is also possible to apply multiple capture zones to create a multiplex test. FIGS. 12 and 17 exemplify a multiplex LFIA in a test strip format. In one embodiment, a diagnostic kit can comprise multiple LFIA test strips, one strip for a different biomarker protein.

In another embodiment, a diagnostic kit can comprise a single composite LFIA test strip for measuring the levels of several biomarker proteins. Such diagnostic kits and LFIA test strips can be used as POCT in the field.

The use of “dip sticks” or LFIA test strips and other solid supports have been described in the art in the context of an immunoassay for a number of antigens. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Three U.S. patents (U.S. Pat. No. 4,444,880, issued to H. Tom; U.S. Pat. No. 4,305,924, issued to R. N. Piasio; and U.S. Pat. No. 4,135,884, issued to J. T. Shen) describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick.

Uses of dipsticks are well known in the art, and are described in U.S. Pat. No. 5,972,594 to Heine, which is incorporated herein in its entirety by reference which is used to detect the presence of neutrophil defensins to diagnose reproductive tract inflammation and preeclampsia.

Other dipsticks and related components are well known in the art, for example dipsticks to detect leukocytes and leukocyte enzymes in body fluids have been patented. For example, U.S. Pat. No. 5,656,448 to Kang et al, which is incorporated herein in its entirety discloses a dipstick encompassed for use in the present invention. Additionally, U.S. Pat. No. 4,758,508 to Schnabel, et al. describes an agent and a method for detecting esterolytic and/or proteolytic enzymes in body fluids. U.S. Pat. No. 4,637,979 to Skjold, et al. describes a composition and test device for measuring the presence of leukocytes in test samples including body fluids such as serum. U.S. Pat. No. 4,645,842 describes pyrrole compounds, and U.S. Pat. No. 4,704,460 (both to Corey) describes novel compounds for detecting the presence of hydrolytic analytes including leukocytes, esterase, and protease, in a test sample, including serum. U.S. Pat. No. 4,774,340 to Corey describes a method for preparing 3-hydroxy pyrroles and esters thereof, which are used to test samples including serum. A composition and test device for measuring the presence of leukocytes, esterase, and protease in a body fluid including serum is described in U.S. Pat. No. 4,657,855 to Corey, et al. A method for measuring the concentration of white blood cells in serum or other biological fluid is described in U.S. Pat. No. 5,663,044 to Noffsinger, et al. A method for preparing an ester used to detect leukocyte cells, esterase, and protease in body fluids such as serum is described in U.S. Pat. No. 4,716,236 to Ward, et al. All of these patents, which are incorporated herein in their entirety by reference, identify an abnormally high level of leukocytes in a patient's serum and produce a signal to identify likelihood that the subject from which the serum was obtained has a pathological condition such as kidney or urogenital tract infection or other dysfunction.

A urine dipstick is a colorimetric chemical assay that can be used to determine the pH, specific gravity, protein, glucose, ketone, bilirubin, urobilinogen, blood, leukocyte, and nitrite levels of an individual's urine. It consists of a reagent stick-pad, which is immersed in a fresh serum specimen and then withdrawn. After predetermined times the colors of the reagent pad are compared to standardized reference charts. One skilled in the art would be able to adapt and modify the design for use with any sample disclosed herein, e.g. plasma, serum, spent tissue culture media, and digested tissue lysate of a tissue biopsy. The dipstick offers an inexpensive and fast method to perform screening of a biological sample, which help in identifying the presence of various diseases or health problems. The dipstick provides a simple and clear diagnostic guideline and can be used in the methods and kits as described herein. Accordingly, one aspect of the presents invention relates to a method for early detection of a fibrotic condition in an individual using a device, such as a dipstick, to test for the increased level of biomarkers as described herein. Dipsticks useful in the present invention can be used to test for at least one biomarker, for example ENO1 or multiple biomarkers, such as any combination selected from the group of ENO1, RCN-3, α-SMA, RCN-1 and PEDF, or alternatively, multiple biomarkers selected from any combination listed in Tables 4 and 5. Combination dipsticks can be used to test for at least two biomarkers selected from the group of ENO1, RCN-3, α-SMA, RCN-1 and PEDF, or alternatively, multiple biomarkers selected from any combination listed in Table 4. Examples of combinations of more than one biomarkers are ENO 1 and PEDF, ENO 1 and RCN-3, ENO1 and α-SMA, ENO1 and RCN-1, RCN-1 and ENO1, PEDF and α-SMA, PEDF and RCN-3, PEDF and RCN-1, RCN-1 and RCN-3, α-SMA and ENO1, RCN-1 and α-SMA, (ENO 1, RCN-3, and α-SMA), (ENO 1, RCN-3, and PEDF), (ENO1, RCN-3, and RCN-1), (RCN-3, α-SMA, and PEDF), (RCN-3, α-SMA, and RCN-1), (RCN-3, α-SMA, and ENO1), (α-SMA, PEDF, and RCN-1), (α-SMA, PEDF, and RCN-3), (α-SMA, PEDF, and ENO1), (PEDF, RCN-1 and ENO1), (PEDF, RCN-1 and RCN-3), (PEDF, RCN-1 and α-SMA), (RCN-1, ENO1 and α-SMA), (ENO1, RCN-3, α-SMA, and PEDF), (RCN-3, α-SMA, PEDF, and RCN-1), (ENO1, RCN-3, α-SMA, and RCN-1), (ENO1, RCN-3, PEDF, and RCN-1), (ENO1, α-SMA, PEDF, and RCN-1) and all five biomarkers: ENO1, RCN-3, α-SMA, PEDF, and RCN-1.

In some embodiments, the present invention provides a LFIA device such as a dipstick to identify biomarkers in a test biological sample. In one embodiment, provided herein is a method for detecting a fibrotic condition using a LFIA device, such as a dipstick, having diagnostic test reagents to detect a fibrotic condition. The diagnostic test reagents react with the test sample, such as serum test sample to produce a change upon contact with the sample. Another embodiment of the invention is a device, such as a dipstick, that has (1) a positive indication for the presence of a fibrotic condition and (2) a negative indication for the absence of a fibrotic condition. The difference between the positive indication and the negative indication is pre-determined.

In some embodiments, the present invention also provides a method for measuring if a subject has a likelihood of having SSc, NSF or a chronic fibrotic condition. In some embodiments, the method begins with obtaining a sample from an individual, such as a symptomatic patient for SSc, NSF or other chronic fibrotic diseases. Symptomatic patients for SSc, NSF or other chronic fibrotic diseases are described herein. Once the sample is obtained, a device having diagnostic test reagents that detect the presence of at least one biomarker, such as ENO1, RCN-3, α-SMA, RCN-1 and PEDF or any biomarkers listed from Tables 4 and 5 is contacted with the sample collected from the individual. Depending on the type of device used, a certain amount of time might have to pass before the device is read. For example, as a general guideline but not as a limitation, when using a MULTISTIX-2 by Bayer Aktiengesellschaft (Fed. Rep. Germany) two minutes pass between the time that the device is contacted with the sample and when it is read to produce an experimental test result. The MULTISTIX-2 dipstick is sold to test serum. The experimental test result is then compared to a pre-determined test results that correspond to the reference levels to indicate either the presence or absence of SSc, NSF or other chronic fibrotic diseases.

In some embodiments, the method to early diagnose of SSc, NSF or other chronic fibrotic diseases in an individual uses a quantitative device (such as, for example, the MULTISTIX-2, MULTISTIX-10, URISTIX-4, or any fibrosis biomarker-detecting device as disclosed herein) or the subject inventive device that has two indications, one for a positive result and one for a negative result. When using such a quantitative device, it produces a range of results. For example, the MULTISTIX-2 produces quantitative results of 0, trace, +1, +2 and +3. Quantitative results also include “Between +1 and +2” and “Between +2 and +3.” A test result of 0, trace, and +1 corresponds to the absence of SSc, NSF or other chronic fibrotic diseases). A test result of “Between +1 and +2”, “Moderate (+2)”, “Between +2 and +3”, and “Large (+3)” corresponds to the presence of SSc, NSF or other chronic fibrotic diseases). The pre-determination is done using a study where the range of the serum biomarker presence is measured based on the range in serum from confirmed SSc, NSF or other chronic fibrotic diseases subjects as compared to the range of serum maker in the serum from healthy (i.e. confirmed non-SSc, NSF or other chronic fibrotic diseases) individuals.

In some embodiments, a device, such as a dipstick immunological device as disclosed herein can includes (1) a matrix (preferably filter paper) with diagnostic test reagents and (2) a mounting substrate (preferably polystyrene film), which typically does not absorb the test (e.g. serum) sample, such that the user can hold onto the substrate without contacting the sample. The device produces a visual change in the matrix upon contact with the serum sample. In some embodiments, the matrix has two indicators—a first that indicates the presence of SSc, NSF or other chronic fibrotic diseases SSc, NSF or other chronic fibrotic diseases and a second that indicates the absence of SSc, NSF or other chronic fibrotic diseases. The first indicator produces a positive test result and the second indicator produces a negative result. The test result is positive when the test result is pre-determined to correspond with a level of the fibrosis biomarker which is indicative of SSc, NSF or other chronic fibrotic diseases. Conversely, a test result is negative when the test result is pre-determined to be below the level of a fibrosis biomarker which indicates the absence of SSc, NSF or other chronic fibrotic diseases. The device, such as a dipstick device measures the presence of SSc, NSF or other chronic fibrotic diseases with the positive test result, and the absence of SSc, NSF or other chronic fibrotic diseases with the negative test result.

In some embodiments, the diagnostic test reagents (e.g. protein-binding agent and biomarker protein) may be associated with the matrix by any physical or chemical means, including, for example impregnation, coating, linking, and covalent attachment. The matrix may take any convenient physical form, such as a card, pad, strip, or dipstick. Such diagnostic test reagents include the compositions of the above-referenced patents, including an ester (preferably a chromogenic ester) and a diazonium salt such as those described in U.S. Pat. No. 4,637,979. Another preferred reagent is a derivatized pyrrole amino acid ester, a diazonium salt, a buffer, and non-reactive ingredients as described in U.S. Pat. Nos. 4,645,842; 4,637,979; 4,657,855; 4,704,460; 4,758,508; and 4,774,340. The preferred amounts of these ingredients is based on dry weight at the time of impregnation and is as follows: about 0.4% w/w derivatized pyrrole amino acid ester, about 0.2% w/w diazonium salt, about 40.9% w/w buffer, and about 58.5% w/w non-reactive ingredients.

In one embodiment, the test reagent, e.g. the anti-antigen (i.e. the biomarker) antibody of the immunoassay is detectably labeled. In some embodiments, the detectable label is selected from a group consisting of enzyme, fluorescent, biotin, gold, latex, hapten and radioisotope labeling. A detectable-hapten includes but is not limited to biotin, fluorescein, digoxigenin, dinitrophenyl (DNP). Other labels include but are not limited to colloidal gold and latex beads. The latex beads can also be colored. Methods of labeling antibodies, antibody-based moiety, or proteins are known in the art, for example, as described in “Colloidal Gold. Principles. Methods and Applications”, Hayat M A (ed) (1989-91). Vols 1-3, Academic press, London; in “Techniques in Immunocytochemistry”, Bullock G R and Petrusz P (eds) (1982-90) Vols 1, 2, 3, and 4, Academic Press, London; in “Principles of Biological Microtechnique”, Baker J R (1970), Methuen, London; Lillie R D (1965), Histopathologic Technique and practical Histochemistry, 3rd ed, McGraw Hill, New York; Berryman M A, et al (1992), J. Histochem Cytochem 40, 6, 845-857, all of which are incorporated herein in their entirety by reference.

In one embodiment, the detectable label is a dye. A “dye” refers to a substance, compound or particle that can be detected, particularly by visual, fluorescent or instrumental means. A dye can be, for example, but is not limited to, a pigment produced as a coloring agent or ink, such as Brilliant Blue, 3132 Fast Red 2R and 4230 Malachite Blue Lake, all available from Hangzhou Hongyan Pigment Chemical Company, China. The “dye” can also be a particulate label, such as, but is not limited to, blue latex beads or gold particles. The particulate labels may or may not be bound to a protein, depending upon if it is desired for the particles to move in the test strip or not. If the particles are to be immobilized in the test strip, the particles may be conjugated to a protein, e.g. the anti-antigen antibody, which in turn is bound to the test strip by either physical or chemical means.

In colloidal gold labeling technique, the unique red color of the accumulated gold label, when observed by lateral or transverse flow along a membrane on which an antigen is captured by an immobilized antibody, or by observation of the red color intensity in solution, provides an extremely sensitive method for detecting sub nanogram quantities of proteins in solution. A colloidal gold conjugate consists of a suspension of gold particles coated with a selected protein or macromolecule (such as an antibody or antibody-based moiety). The gold particles may be manufactured to any chosen size from 1-250 nm. This gold probe detection system, when incubated with a specific target, such as in a tissue section, will reveal the target through the visibility of the gold particles themselves. For detection by eye, gold particles will also reveal immobilized antigen on a solid phase such as a blotting membrane through the accumulated red color of the gold sol. Silver enhancement of this gold precipitate also gives further sensitivity of detection. Suppliers of colloidal gold reagents for labeling are available from SPI-MARK™. Polystyrene latex Bead size 200 nm colored latex bead coated with antibody SIGMA ALDRICH®, Molecular Probes, Bangs Laboratory Inc., and AGILENT® Technologies.

Other detection systems can also be used, for example, a biotin-streptavidin system. In this system, the antibodies immunoreactive (i.e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e.g. from DAKO; Carpinteria, Calif.

Protein binding agents described herein such as antibodies and antibody-based moiety can alternatively be labeled with any of a number of fluorescent compounds such as fluorescein isothiocyanate, europium, lucifer yellow, rhodamine β isothiocyanate (Wood, P. In: Principles and Practice of Immunoasay, Stockton Press, New York, pages 365-392 (1991)) for use in immunoassays. In conjunction with the known techniques for separation of antibody-antigen complexes, these fluorophores can be used to quantify the biomarker of interest. The same applies to chemiluminescent immunoassay in which case antibody or biomarker of interest can be labeled with isoluminol or acridinium esters (Krodel, E. et al., In: Bioluminescence and Chemiluminescence: Current Status. John Wiley and Sons Inc. New York, pp 107-110 (1991); Weeks, I. et al., Clin. Chem. 29:1480-1483 (1983)). Radioimmunoassay (Kashyap, M. L. et al., J. Clin. Invest, 60:171-180 (1977)) is another technique in which antibody can be used after labeling with a radioactive isotope such as ¹²⁵I. Some of these immunoassays can be easily automated by the use of appropriate instruments such as the IMX™ (Abbott, Irving, Tex.) for a fluorescent immunoassay and Ciba Coming ACS 180™ (Ciba Corning, Medfield, Mass.) for a chemiluminescent immunoassay.

A “LFIA test strip” or “dip stick” can include one or more bibulous or non-bibulous materials or matrices. In reference to a “LFIA test strip” or “dip stick”, the terms “material” and “matrix” are used interchangeably. If a test strip comprises more than one material, the one or more materials are preferably in fluid communication. One material of a test strip may be overlaid on another material of the test strip, such as for example, filter paper overlaid on nitrocellulose membrane. Alternatively or in addition, a test strip can include a region comprising one or more materials followed by a region comprising one or more different materials. In this case, the regions are in fluid communication and may or may not partially overlap one another. Suitable materials for test strips include, but are not limited to, materials derived from cellulose, such as filter paper, chromatographic paper, nitrocellulose, and cellulose acetate, as well as materials made of glass fibers, nylon, dacron, PVC, polyacrylamide, cross-linked dextran, agarose, polyacrylate, ceramic materials, and the like. The material or materials of the test strip may optionally be treated to modify their capillary flow characteristics or the characteristics of the applied sample. For example, the sample application region of the test strip may be treated with buffers to correct the pH or specific gravity of an applied serum sample, to ensure optimal test conditions.

The material or materials can be a single structure such as a sheet cut into strips or it can be several strips or particulate material bound to a support or solid surface such as found, for example, in thin-layer chromatography and may have an absorbent pad either as an integral part or in liquid contact. The material can also be a sheet having lanes thereon, capable of spotting to induce lane formation, wherein a separate assay can be conducted in each lane. The material can have a rectangular, circular, oval, triagonal or other shape provided that there is at least one direction of traversal of a test solution by capillary migration. Other directions of traversal may occur such as in an oval or circular piece contacted in the center with the test solution. However, the main consideration is that there is at least one direction of flow to a predetermined site.

The support for the test strip, where a support is desired or necessary, will normally be water insoluble, frequently non-porous and rigid but may be elastic, usually hydrophobic, and porous and usually will be of the same length and width as the strip but may be larger or smaller. The support material can be transparent, and, when a test device is assembled, a transparent support material can be on the side of the test strip that can be viewed by the user, such that the transparent support material forms a protective layer over the test strip where it may be exposed to the external environment, such as by an aperture in the front of a test device. A wide variety of materials, both natural and synthetic, and combinations thereof, may be employed provided only that the support does not interfere with the capillary action of the material or materials, or non-specifically bind assay components, or interfere with the signal producing system. Illustrative polymers include polyethylene, polypropylene, poly(4-methylbutene), polystyrene, polymethacrylate, poly(ethylene terephthalate), nylon, poly(vinyl butyrate), glass, ceramics, metals, and the like. Elastic supports may be made of polyurethane, neoprene, latex, silicone rubber and the like.

In some embodiments, a dipstick device has one indication of the presence of SSc, NSF or other chronic fibrotic diseases and a second indication for the absence of SSc, NSF or other chronic fibrotic diseases. The two indications preferably are a negative (−) symbol and a positive (+) symbol, but could be any two indications. In one embodiment, the device has the negative indication (e.g., the “−” portion of a possible “+” symbol) containing reagents that react with all samples. That is, the diagnostic test reagents react to some constituent analyte, such as urea which is present in all serum samples. Alternatively, the diagnostic test reagents test an aspect of the sample, such as pH, that every sample has. The positive indication (e.g., the “|” portion of a “+” symbol) contains a reagent that the reacts only with a sample containing the presence of a test biomarker which is above a certain pre-defined level, such that it reacts in serum samples which only contain the presence of the biomarker (i.e. of a ENO1 biomarker) above a certain level, i.e. above a pre-defined level of the biomarker. Another embodiment has the negative indicator (e.g., the “−” portion of a possible “+” symbol) which contains reagents that reacts with the sample which either has the absence of the test fibrotic disease biomarker (i.e. absence of the ENO1 biomarker) or the level of the test fibrotic disease biomarker (i.e. the ENO1 biomarker) below a certain pre-defined or threshold level. The positive indication (e.g., the “|” part of the “+” symbol) has a lower sensitivity to the presence of a test fibrotic disease biomarker (i.e. ENO1 biomarker) and thus such the reagents react only with samples containing level of the biomarker (i.e. LRG biomarker) above a pre-defined level, which in one embodiment is the average plus two standard deviation of the statistics obtained for a population of healthy individuals.

In some embodiments, a test device, such as a dipstick device has text on the device in two places. In one place the text indicates a positive result (i.e. the likelihood the subject has SSc, NSF or other chronic fibrotic diseases). In another, it indicates a negative result (i.e. the likelihood the subject does not have SSc, NSF or other chronic fibrotic diseases). Next to the indications are matrices having the appropriate diagnostic test reagents. For example, next to the negative indication is a matrix having diagnostic test reagents that react with all samples, regardless of the content of biomarkers as disclosed herein. Next to the positive indication is a matrix having diagnostic test reagents that react only with samples that have the presence of the test fibrotic disease biomarker, (e.g. ENO1, or any or any combination of fibrosis disease biomarkers listed in Tables 4 and 5) above a pre-defined level. In some embodiments, such devices such as those discussed in FIG. 9-17, do not require a chart, such as a coloration chart, to interpret the results. In some embodiments of this aspect of the invention, this enables the detection device, such as a dipstick device (and the corresponding method) to be used easily by one without special training and provides a more rapid diagnostic (and method) for measuring if a subject is likely to have SSc, NSF or other chronic fibrotic diseases. In some embodiments of this aspect of the invention, such a device is ideal for point-of-care testing (POCT) application.

Production and manufacturer of dipsticks are well known by ordinary skill in the art. Dipsticks are commercially available from Bayer Corporation of Elkhart, Ind., as well as other commercial sources. The dipstick is dipped into a well mixed biological sample, e.g. serum, and after a time period, for example between about thirty seconds (30 s) to about two minutes (2 mins) or more, the various reagent bands are visually or optically examined for color changes. The bands can be visually compared to a preprinted color chart in order to determine the amount of each of the constituents or parameters being measured. It is also possible to optically scan using a machine or optical scanner the dipstick and thereby obtain instrument readings of color intensity or wave length through the use of a particular instrument adapted for reading the reagents and color of the dipstick. Examples of such instruments or machines are manufactured by Ames. Examples of useful machines or instruments for optically scanning the dipstick bands are able to distinguish between positive and negative reaction or reagent bands, was well as differences in color distribution of the reagent bands in the presence (i.e. above a certain threshold level) or absence (or below a certain threshold level) of the test fibrotic disease biomarker(s). In some embodiments, the instrument is capable of quantify a number of reagent bands as well as quantify the overall color intensity sensed on the band.

In some embodiments, the immunoassays operate on a purely qualitative basis. However, it is possible to measure the intensity of the test line to measure the quantity of antigen in the sample when using an immunoassay such a LFIA. Implementing a magnetic immunoassay (MIA) in the lateral flow test form also allows for getting a quantified result.

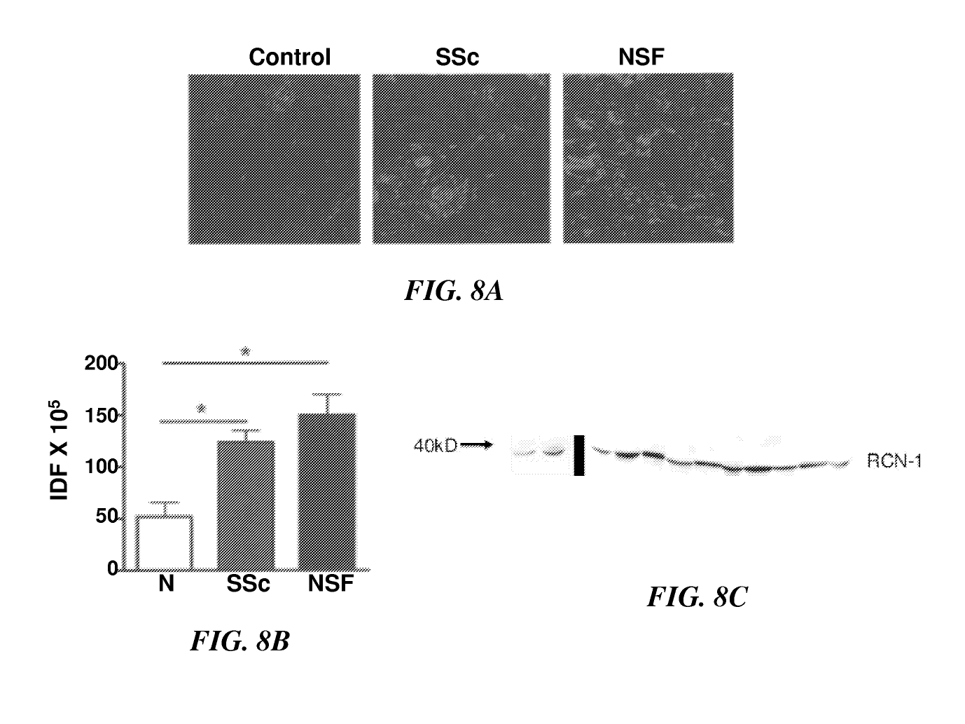

Instruments have been developed which both determine the chemical constituents of the serum and also assist in the microscopic analysis, for example, the instrument disclosed in U.S. Pat. No. 6,004,821 which is incorporated herein in its entirety by reference. Such an instrument is the Yellow IRIS, which automatically places the sample on the serum dipstick and then reads the chemical results. FIG. 8 of U.S. Pat. No. 6,004,821 shows a schematic depiction of such an automated calorimetric microscopical instrument assembly (which is denoted generally by the numeral 54), and which can be used to scan a serum sample, and can, without significant human intervention, colorometrically analyze the wavelengths of the colors imparted to the dipstick by the serum in the chamber 14, either colorometrically and/or morphometrically. Accordingly, such an instrument, which is specifically adapted to scan the reaction of the dipstick after contact with an appropriate sample for the presence of the fibrosis biomarkers (e.g. those selected from Tables 4 and 5) is encompassed for use in the present invention.

In some embodiments, the dipstick uses reagents such as copper-creatinine and iron-creatinine complexes have peroxidase activity. Other dipstick reagents can use reagents such as 3,3′,5,5′-tetramethylbenzidine (TMB), and diisopropyl benzene dihydroperoxide (DBDH) which are used with peroxidase. In some embodiments, a dipstick for use to detect the presence of fibrosis biomarkers is based upon the first-generation devices which relied on the same colorimetric reaction used for assessing the presence of glucose test strips for serum. Besides glucose oxidase, a test kit for use herein can contain a benzidine derivative, which is oxidized to a blue polymer by the hydrogen peroxide formed in the oxidation reaction. Care must be taken if such a dipstick is generated to ensure the test strip is developed after a precise interval after contact with the serum test sample as well as frequent calibration of the meter to read the test result. The same principle is used in test strips that have been commercialized for the detection of diabetic ketoacidosis (DKA). These test strips use a beta-hydroxybutyrate-dehydrogenase enzyme instead of a glucose oxidizing enzyme and have been used to detect and help treat some of the complications that can result from prolonged hyperglycaemia. Blood alcohol sensors using the same approach but with alcohol dehydrogenase enzymes have been developed.

In another embodiment, the device, such as a dipstick device uses an electrochemical method. Test strips contain a capillary that sucks up a reproducible amount of serum. The fibrosis biomarker, such as any or a combination of those listed in Tables 4 and 5, in a biological sample reacts with an enzyme electrode containing protein-binding agents. The coulometric method is a technique where the total amount of charge generated by the specific binding of the fibrosis biomarker to the specific protein-binding agent reaction is measured over a period of time. This is analogous to throwing a ball and measuring the distance it has covered so as to determine how hard it was thrown. The amperometric method is used by some meters and measures the electrical current generated at a specific point in time. This is analogous to throwing a ball and using the speed at which it is travelling at a point in time to estimate how hard it was thrown. The coulometric method can allow for variable test times, whereas the test time on a meter using the amperometric method is always fixed. Both methods give an estimation of the concentration of the fibrosis biomarker in the sample.

In one embodiment, the levels of biomarker proteins in a biological sample are detected by a magnetic immunoassay (MIA). MIA is a type of diagnostic immunoassay using magnetic beads as labels in lieu of conventional enzymes (ELISA), radioisotopes (RIA) or fluorescent moieties (fluorescent immunoassays). This assay involves the specific binding of a protein binding agent to a biomarker protein, such as an antibody binding to its antigen, where a magnetic label is conjugated to one element of the pair. The presence of magnetic beads is then detected by a magnetic reader (magnetometer) which measures the magnetic field change induced by the beads. The signal measured by the magnetometer is proportional to the antigen or biomarker quantity in the initial sample.

Magnetic beads are made of nanometric-sized iron oxide particles encapsulated or glued together with polymers. These magnetic beads can range from 35 nm up to 4.5 μm. The component magnetic nanoparticles range from 5 to 50 nm and exhibit a unique quality referred to as superparamagnetism in the presence of an externally applied magnetic field. Magnetic labels exhibit several features very well adapted for such applications: they are not affected by reagent chemistry or photo-bleaching and are therefore stable over time; the magnetic background in a biomolecular sample is usually insignificant; sample turbidity or staining have no impact on magnetic properties; and magnetic beads can be manipulated remotely by magnetism.

The use of MIA is well known in the art, for example, Dittmer W U and colleagues (J Immunol Methods. 2008, 338:40-6) described a sensitive and rapid immunoassay for detection and measurement parathyroid hormone using magnetic particle labels and magnetic actuation. The assay involves a 1-step sandwich immunoassay with no fluid replacement steps. The detection limit is the 10 pM range and the assay took only 15 minutes; Kuma H and colleagues (Rinsho Byori. 2007, 55:351-7) developed a sensitive immunoassay system using magnetic nanoparticles made from Fe3O4; and Kuramitz H. reviews the current state of concerning electrochemical immunoassays using magnetic microbeads as a solid phase in Anal Bioanal Chem. 2009, 394:61-9. U.S. Pat. Nos. 5,252,493; 5,238,811; 5,236,824; 7,604,956; U.S. Patent Application No. 20090216082; 20090181359; and 20090263834 all describe various improvements and versions of MIA. These references are all incorporated herein in their entirety by reference.

Magnetometers are instruments that can detect the presence and measure the total magnetic signal of a sample. An effective MIA is one that is capable of separating naturally occurring magnetic background (noise) from the weak magnetically labeled target (signal). Various approaches and devices have been employed to achieve a meaningful signal-to-noise ratio (SNR) for bio-sensing applications: giant magneto-resistive sensors and spin valves, piezo-resistive cantilevers, inductive sensors, superconducting quantum interference devices, anisotropic magneto-resistive rings, and miniature Hall sensors. MIA that exploits the non-linear magnetic properties of magnetic labels can effectively use the intrinsic ability of a magnetic field to pass through plastic, water, nitrocellulose, and other materials, thus allowing for true volumetric measurements in various immunoassay formats. Unlike conventional methods that measure the susceptibility of superparamagnetic materials, a MIA based on non-linear magnetization eliminates the impact of linear dia- or paramagnetic materials such as sample matrix, consumable plastics and/or nitrocellulose. Although the intrinsic magnetism of these materials is very weak, with typical susceptibility values of −10-5 (dia) or +10-3 (para), when one is investigating very small quantities of superparamagnetic materials, such as nanograms per test, the background signal generated by ancillary materials cannot be ignored. In MIA based on non-linear magnetic properties of magnetic labels the beads are exposed to an alternating magnetic field at two frequencies, f1 and f2. In the presence of non-linear materials such as superparamagnetic labels, a signal can be recorded at combinatorial frequencies, for example, at f=f1±2×f2. This signal is exactly proportional to the amount of magnetic material inside the reading coil. Ultrasensitive magnetic biosensor for homogeneous immunoassay has been described by Y. R. Chemla, et al., Proc. Natl. Acad. Sci. U.S.A. 2000, 97:14268-14272. This is incorporated herein in its entirety by reference.

In one embodiment, the levels of biomarker proteins in a sample are detected by a diffusion immunoassay (DIA). In this assay, the transport of molecules perpendicular to flow in a microchannel, e.g. in a microfluidic chip, is affected by binding between antigens and antibodies. By imaging the steady-state position of labeled components in a flowing stream, the concentration of very dilute analytes, in this invention, the biomarkers, can be measured in a few microliters of sample in seconds. Microfluidics is the manipulation of microliter volumes in channels with sub-millimeter dimensions. Microfluidic diffusion immunoassays for the detection of analytes or biomarkers in fluid samples have been described in the art, for example, in U.S. Pat. Nos. 6,541,213; 6,949 377; 7,271,007; U.S. Patent Application No. 20090194707; 20090181411; in Hatch et al., 2001, Nature Biotechnology 19(5): 461-465; K. Scott Phillips and Quan Cheng, Anal. Chem., 2005, 77:327-334; J. Hsieh, et al., Nanotech 2007 Vol. 3, Technical Proceedings of the 2007 NSTI Nanotechnology Conference and Trade Show, Chapter 4: Micro and Nano Fluidics, pp 292-295; Frank Y. H. Lin et al., Clinical and Diagnostic Laboratory Immunology, 2005, 12:418-425; and A. Bhattacharyya and C. M. Klapperich, 2007, Biomedical Microdevices, 9: 245-251. These are incorporated herein in their entirety by reference. U.S. Pat. No. 6,541,213 describes the use of a credit-card sized microfluidic device to perform competitive immunoassays. The ability to perform assays in this microscale dimension affords an extremely rapid, homogenous, and cost effective alternative to current methods used commercially today. The credit-card sized microfluidic device can be integrated into the development of point-of-use systems that allow real-time answers to health questions while at the physician's office, home, workplace, school, shopping mall and other public places. These systems include portable and handheld instruments with integrated laboratory-tests-on-a-card (“lab cards”), as well as stand alone, single use lab cards being developed to provide rapid on-site results in infectious diseases testing, nucleic acid testing, blood type analysis, cancer testing, and respiratory disease testing.

In one embodiment, the levels of biomarker proteins in a sample are detected by an on-the-spot assay also known as point-of-care assay. Point-of-care testing (POCT) is defined as diagnostic testing at or near the site of patient care. Currently majority of the detection and diagnostic testing for analytes, toxin, pathogen toxins and antigens in samples are largely restricted to centralized laboratories because of the need for long assay times, complex and expensive equipment, and highly trained technicians. POCT brings the test conveniently and immediately to the patient. This increases the likelihood that the patient will receive the results in a timely manner. POCT is accomplished through the use of transportable, portable, and handheld instruments (e.g., blood glucose meter, nerve conduction study device) and test kits (e.g., CRP, HBA1C, Homocystein, HIV salivary assay, etc.). POCTs are well known in the art, especially immunoassays. For example, the LFIA test strip or dip sticks can easily be integrated into a POCT diagnostic kit. One skilled in the art would be able to modify immunoassays for POCT using different format, e.g. ELISA in a microfluidic device format or a test strip format. For example, U.S. Patent Application No. 2009/0181411 describes a microfluidic device-based point-of-care immunoassay for biomarker molecules associated with pathology in a vertebrate host, man or animal. The microfluidic devices such as chips are formatted to either hand-held cartridges (also termed “cards”), or cartridges for automated or semi-automated, machine-aided testing. Microfluidic device-based assays enable small-volume sampling, with point-of-care results from a broad variety of biological fluids and samples in real time. In addition, the assay cartridges can be single use reagent packs, or be fully self-contained and operable entirely by hand. This reference is incorporated herein in its entirety by reference.

Embodiments of the invention further provide for diagnostic kits and products of manufacture comprising the diagnostic kits. The kits can comprise a means for predicting the presence or likelihood of SSc, NSF or other chronic fibrotic condition in an individual, preferably a human.

In one embodiment, the kit comprises an indicator responsive to the level of biomarker protein in a biological sample, e.g. serum, wherein the biomarker protein is selected from the group consisting of ENO1, RCN-3, α-SMA, RCN-1 and PEDF, or any combinations of biomarkers selected from those listed in Tables 4 and 5. In some embodiments, the indicator is in the form of a LFIA test strip or a microfluidic device. In one embodiment, a diagnostic kit can comprise multiple LFIA test strips, one strip for a different biomarker protein. In another embodiment, a diagnostic kit can comprise a single composite LFIA test strip for measuring the levels of several biomarker proteins. In one embodiment, a diagnostic kit can comprise a single multichannel microfluidic device for measuring the levels of several biomarker proteins. In another embodiment, a diagnostic kit can comprise several microfluidic devices for measuring the levels of several biomarker proteins, one microfluidic device for a different biomarker protein.

The kits can further comprise cups or tubes, or any other collection devices for the collection of biological samples and their preparation thereof (e.g. lysate of tissue biopsy or culturing ex vivo the tissue excised from an individual.

In one embodiment, the kit can optionally further comprise at least one diagram and/or instructions describing the interpretation of test results.

In one embodiment, the biomarker proteins described herein and/or its mRNA levels in a sample can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated in their entirety by reference.

Mass spectrometry methods are well known in the art and have been used to quantify and/or identify biomolecules, such as proteins (see, e.g., Li et al., 2000, Tibtech 18:151-160; Rowley et al., 2000, Methods 20: 383-397; and Kuster and Mann, 1998, Curr. Opin. Structural Biol. 8: 393-400. Further, mass spectrometric techniques have been developed that permit at least partial de novo sequencing of isolated proteins, see Chait et al., 1993, Science, 262:89-92; Keough et al., 1999 Proc. Natl. Acad. Sci. USA. 96:7131-6; and eviewed in Bergman, 2000, EXS 88:133-44.

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15, John Wiley & Sons, New York 1995, pp. 1071-1094.

Detection and quantification of the biomarker proteins will typically depend on the detection of signal intensity. This, in turn, can reflect the quantity and character of a protein bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Measuring Biomarker Protein Expression Level by Measuring mRNA

In one embodiment, the mRNA of RCN-1, ENO1, PEDF or any of the biomarker proteins of Tables 4 and 5 can be measured by quantitative real-time PCR. Real time PCR is an amplification technique that can be used to measure levels of mRNA expression. (See, e.g., Gibson et al., 1996, Genome Research 6:995-1001, Heid et al., 1996, Genome Research 6:986-994). Real-time PCR evaluates the level of PCR product accumulation during amplification. This technique permits quantitative evaluation of mRNA levels in multiple samples. For mRNA levels, mRNA is extracted from a biological sample, e.g. a tumor and normal tissue, and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for the genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes can be obtained commercially from, for example, Perkin Elmer/Applied Biosystems (Foster City, Calif.). To quantify the amount of the specific nucleic acid of interest in a sample, a standard curve is generated using a control. Standard curves can be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

The TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification.

Total RNA can be prepared by any methods known in the art, for example, isolation by centrifugation through spin columns using the SV Total RNA Isolation System (Promega). cDNA can be generated form total RNA (4 μg total cellular RNA per reaction) by reverse transcription using Oligo (dT) 15 primer (Promega) and SUPERSCRIPT III (500 units per reaction; Invitrogen) for first strand synthesis and Tfl DNA polymerase for second strand synthesis (500 units per reaction; Promega) as per the manufacturer's protocol. Typically, 20 pg of each cDNA is used for each PCR reaction. Two-step RT-qPCR is performed on cDNA generated using the Multi-Scribe reverse transcriptase from the TaqMan reverse transcription system and the SYBR Green PCR Master Mix (PE Biosystems). The primer pairs for RT-qPCR of the biomarkers can be designed by any methods known in the art, e.g. the published GENBANK™ sequences. For example, the RCN-1 forward primer 5′-TGCAGACCTCAATGGTGACCT-3′ (SEQ. ID. No. 12) and RCN-1 reverse primer 5′-AAGGCAGTGAACTCCTCCCG-3′ (SEQ. ID. No. 13). ENO1 forward primer 5′-GAGCTCCGGGACAATGATAA-3′ (SEQ. ID. No. 14) and ENO1 reverse primer 5′-TGTTCCATCCATCTCGATCA-3′ (SEQ. ID. No. 15). PEDF forward primer 5′-TGTGCAGGCTTAGAGGGACT-3′ (SEQ. ID. No. 16) and PEDF reverse primer 5′-GTTCACGGGGACTTTGAAGA-3′ (SEQ. ID. No. 17). RCN3 forward primer 5′-GACTTCCGGGATCTGAACAA-3′ (SEQ. ID. No. 18) and RCN3 reverse primer 5′-CCATAGTTGGTGGCCTGACT-3′ (SEQ. ID. No. 19). α-SMA forward primer 5′-TTCAATGTCCCAGCCATGTA-3′ (SEQ. ID. No. 20) and α-SMA reverse primer 5′-GAAGGAATAGCCACGCTCAG-3′ (SEQ. ID. No. 21). Reactions are performed in MicroAmp Optical 96-well Reaction Plate (PE Biosystems). Forty cycles of PCR are performed under standard conditions using an annealing temperature of 60° C. The cycle threshold (Ct) is determined by the cycle number at which exponential amplification began. The Cts are averaged from the values obtained from the triplicate repeats used to calculate delta Ct normalized to β-actin. It is recommended that all reactions are carried out in triplicates.

Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. Detailed protocols for quantitative PCR are provided, for example, in Innis et al., 1990, PCR Protocols, A Guide to Methods and Applications, Academic Press, Inc. N.Y.

In another embodiment, the detection of mRNA transcripts of the biomarker proteins can be achieved by Northern blotting, wherein a preparation of RNA is electrophoretically separated on a denaturing agarose gel, and transferred to a suitable support, such as activated cellulose, nitrocellulose or glass or nylon membranes. Northern blotting is well known in the art. Labeled (e.g., radiolabeled) cDNA or RNA or short probes corresponding to e.g. ENO1, RCN-1, or PEDF cDNAs are then hybridized to the preparation, washed and analyzed by methods such as autoradiography. In some embodiments, the PEDF probe is 5′-TGTGCAGGCTTAGAGGGACT-3′ (SEQ. ID. No. 22), the RCN-1 probe is 5′-GGAGTTCACTGCCTTTCTGC-3′ (SEQ. ID. No. 23), the RCN3 probe is 5′-AGCGGAAATCCTGGGTAATT-3′ (SEQ. ID. No. 24), the α-SMA probe is 5′-ACCCACAATGTCCCCATCTA-3′ (SEQ. ID. No. 25), and the ENO1 probe is 5′-GAAGGGTGTCTCAAAGGCTG-3′ (SEQ. ID. No. 26).

Alternatively, mRNA expression can be detected on a DNA array, chip or a microarray. Oligonucleotides corresponding to the biomarkers cDNA are immobilized on a chip which is then hybridized with labeled nucleic acids derived from a sample obtained from a patient. Positive hybridization signal is obtained with the sample containing biomarkers transcripts. Methods of preparing DNA arrays and their use are well known in the art. (See, for example U.S. Pat. Nos. 6,379,897; 6,664,377; 6,451,536; and 548,257; U.S. 20030157485 and Schena et al., 1995, Science 20:467-470; Gerhold et al., 1999, Trends in Biochem. Sci. 24, 168-173; and Lennon et al., 2000, Drug discovery Today 5: 59-65, which are herein incorporated herein in their entirety by reference). Serial Analysis of Gene Expression (SAGE) can also be performed (e.g. U.S. Patent Application 20030215858).

To monitor mRNA levels, for example, mRNA is extracted from the sample to be tested, reverse transcribed, and fluorescent-labeled cDNA probes are generated. The microarrays capable of hybridizing to biomarkers cDNA are then probed with the labeled cDNA probes, the slides scanned and fluorescence intensity measured. This intensity correlates with the hybridization intensity and expression levels. Quantitative microarray hybridization assays are well known in the art, for example, in U.S. Pat. Nos. 6,004,755 and 6,492,122, both of which are incorporated herein in their entirety by reference.

### Antibodies or Antisera Against the Biomarkers

In one embodiment, the methods disclosed herein uses antibodies, antibody-binding moiety or anti-sera for detecting, quantifying, and/or labeling the levels of the biomarker proteins described herein. The antibodies can be obtained from a commercial source such as GenWay Biotech Inc. catalog No: 18-003-42514, ABCAM® catalog No. ab54979, Cosmo Bio Com. Ltd. catalog No: CAC-SK-T01-013, Sigma Aldrich catalog No. A2547 and Santa Cruz Biotechnologies, Santa Cruz, Calif.; catalog No: sc-53921. These commercial antibodies can also be conjugated with labels, e.g. Cy 3 (Sigma Aldrich catalog No. C6198) or FITC (Sigma Aldrich catalog No. F3777).

Antibodies for use in the methods described herein can also be produced using standard methods to produce antibodies, for example, by monoclonal antibody production (Campbell, A.M., Monoclonal Antibodies Technology Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

Methods for the production of antibodies are disclosed in PCT publication WO 97/40072 or U.S. Application. No. 2002/0182702, which are herein incorporated by reference. The processes of immunization to elicit antibody production in a mammal, the generation of hybridomas to produce monoclonal antibodies, and the purification of antibodies may be performed by described in “Current Protocols in Immunology” (CPI) (John Wiley and Sons, Inc.) and Antibodies: A Laboratory Manual (Ed Harlow and David Lane editors, Cold Spring Harbor Laboratory Press 1988) which are both incorporated by reference herein in their entireties; —Brown, “Clinical Use of Monoclonal Antibodies,” in BIOTECHNOLOGY AND PHARMACY 227-49, Pezzuto et al. (eds.) (Chapman & Hall 1993).

For example, to generate a polyclonal antibody against human RCN-3, recombinant RCN-3 possessing an amino-terminal hexa-histidine tag can be prepared as follows. Full-length RCN-3 cDNA (Genbank Accession No. NM_{—}020650.2) is cloned into the pQE30 vector containing an N-terminal hexa-histidine tag (Qiagen, GmbH, Hilden, Germany), and then transformed into E. coli strain JM109 cells. RCN-3 was expressed and purified by affinity chromatography using Ni-nitriloacetic acid agarose (Qiagen) according to the manufacturer's instructions. The final preparation yielded a single 36 kDa band on SDS-PAGE and is used for the immunization of rabbits.

### Computer Systems and Computer Readable Media to Assay Fibrotic Biomarkers in Samples

Embodiments of the invention also provide for systems (and computer readable media for causing computer systems) to perform a method for early diagnosis SSC, NSF or other chronic fibrotic condition in an individual, assessing an individual's likelihood of having SSC, NSF or other chronic fibrotic condition, or monitoring treatment efficacy of an individual with SSC, NSF or other chronic fibrotic condition.

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

One aspect of the present invention relates to a system for analyzing a biological sample from a individual, where the system comprises: (a) a determination module configured to receive a biological sample and to measure a biomarker level information, wherein the biomarker level information comprises measurement of at least one biomarker level, i.e. the level or amount of a biomarker, such as, or any ENO1, RCN-3, α-SMA, RCN-1 and PEDF or a combination of biomarkers listed in Table 4; (b) a connection from the determination module to transmit the biomarker level information to an electronic computer, wherein the computer comprises a storage device, a comparison module and a display module; (c) the storage device configured to store the biomarker level information from the determination module; (d) the comparison module adapted to compare the biomarker level information stored on the storage device with reference data, and to provide a comparison result, wherein the comparison result comprises; (i) a comparison of the biomarker level in the sample with the reference biomarker level, and (ii) a measurement of the biomarker level in the sample above or below a threshold level relative to the reference biomarker level, wherein a biomarker level above the threshold level for that biomarker is indicative of acute (i.e. a positive test result); and wherein a biomarker level below the threshold level is indicative of absence of a fibrotic condition (i.e. a negative test result); and (e) the display module for displaying a content based in part on the comparison result for the user, wherein the content is a signal indicative of the likelihood of a subject having SSc, NSF or other chronic fibrotic condition (i.e. a positive test result) or unlikely to have SSc, NSF or other chronic fibrotic condition (i.e. a negative test result).

Another aspect of the present invention relates to a computer readable medium having computer readable instructions recorded thereon to define software modules including a comparison module and a display module for implementing a method on a computer, the method comprising: (a) comparing with the comparison module the data stored on a storage device with reference data to provide a comparison result, wherein the comparison result is that the biomarker level information in the biological sample is above a threshold level relative to a reference biomarker level for that biomarker tested and that is indicative of SSc, NSF or other chronic fibrotic disease; and (b) displaying a content based in part on the comparison result for the user, wherein the content is a signal indicative of SSc, NSF or other chronic fibrotic disease.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention include at minimum a determination system, a storage device, a comparison module, and a display module. The functional modules can be executed on one, or multiple, computers, or by using one, or multiple, computer networks. The determination system has computer executable instructions to provide e.g., fibrotic biomarker level information in computer readable form.

The determination system can comprise any system for detecting a signal representing level of a biomarker. Such systems can include DNA microarrays, RNA expression arrays, any ELISA detection system and/or any Western blotting detection system.

The information determined in the determination system can be read by the storage device. As used herein the “storage device” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon expression level or protein level information. Such information may be provided in digital form that can be transmitted and read electronically, e.g., via the Internet, on diskette, via USB (universal serial bus) or via any other suitable mode of communication.

As used herein, “stored” refers to a process for encoding information on the storage device. Those skilled in the art can readily adopt any of the presently known methods for recording information on known media to generate manufactures comprising expression level information.

In one embodiment, the reference data stored in the storage device to be read by the comparison module is e.g., biomarker level data obtained from a population of healthy individuals who do not have SSc, NSF or and other chronic fibrotic disease. The data form the healthy population can be obtained using the determination system. When the system is used for determining the efficacy of a treatment regime, the reference data stored in the storage device to be read by the comparison module can be biomarker level data from the individual obtained at a prior time point using the determination module.

The “comparison module” can use a variety of available software programs and formats for the comparison operative to compare expression data determined in the determination system to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related to normalized level of a fibrosis biomarker, the likelihood of the presence/absence of SSC, NSF or other chronic fibrotic condition in an individual, efficacy of treatment in an individual, and/or method for treating an individual.

The comparison module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GENBANK™ or Swiss Pro World Wide Web site). Thus, in a particular preferred embodiment of the present invention, users can directly access data residing on Internet databases using a HTML interface provided by Web browsers and Web servers.

The comparison module provides a computer readable comparison result that can be processed in computer readable form by predefined criteria, or criteria defined by a user, to provide a content-based in part on the comparison result that may be stored and output as requested by a user using a display module.

The content based on the comparison result, which may be a biomarker level value compared to a reference, can show the likelihood of the presence/absence of SSC, NSF or other chronic fibrotic disease in an individual or an assessed the treatment efficacy of SSC, NSF or other chronic fibrotic disease in an individual.

In one embodiment of the invention, the content based on the comparison result is displayed on a computer monitor. In one embodiment of the invention, the content based on the comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

Systems and computer readable media described herein are merely illustrative embodiments of the invention for performing methods of assessing SSc, NSF or other chronic fibrotic disease status in an individual, and are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

In some embodiments, the biomarker threshold level which is used in the system, computer-readable medium and methods as disclosed herein that is indicative of SSc, NSF or other chronic fibrotic disease is at a level of at least about two standard deviations above the control or reference biomarker level for that biomarker. For example, if the biomarker is RCN-3, if the level of RCN-3 in the biological sample from the individual is at least about two standard deviations above the reference RCN-3 biomarker level, it is indicative of the individual is likely to have or be at risk of SSc, NSF or other chronic fibrotic disease. In some embodiments, a threshold level is at least about 3 standard deviations, or at least about 4 standard deviations, or at least about 5 standard deviations, or at least about 6 standard deviations, or at least about 7 standard deviations or at least about 8 standard deviations, or at least about 9 standard deviations, or at least about 10 standard deviations or more than 10 standard deviations above the reference level for that biomarker, and thus a level of the biomarker in the biological sample above the threshold level it is indicative of a subject likely to have SSc, NSF or other chronic fibrotic disease.

In some embodiments, the system, computer-readable media and methods as disclosed herein is used to measure a biomarker level in a sample, where the biomarker level is the amount of a biomarker protein, for example any biomarker listed in Tables 4 and 5 or of any of the protein sequences having SEQ. ID NOS: 1-11. In some embodiments, the level of at least one biomarker protein is measured by immunoassay, for example western blot analysis or ELISA, or a high through-put protein detection method, for example but are not limited to automated immunohistochemistry apparatus, for example, robotically automated immunohistochemistry apparatus which in an automated system section the tissue or biological sample specimen, prepare slides, perform immunohistochemistry procedure and detect intensity of immunostaining, such as intensity of an antibody binding to a biomarker protein in the serum sample and produce output data. Examples of such automated immunohistochemistry apparatus are commercially available, for example such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839; 091 which are incorporated herein in their entirety by reference. Other commercially available automated immunohistochemistry instruments are also encompassed for use in the present invention, for example, but not are limited BOND™ Automated Immunohistochemistry & In Situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other determination module can be an automated immunohistochemistry systems such as NEXES® automated immunohistochemistry (IHC) slide staining system or BENCHMARK® LT automated IHC instrument from Ventana Discovery SA, which can be combined with VIAS™ image analysis system also available Ventana Discovery. BioGenex Super Sensitive MULTILINK® Detection Systems, in either manual or automated protocols can also be used as the detection module, preferably using the BioGenex Automated Staining Systems. Such systems can be combined with a BioGenex automated staining systems, the i6000™ (and its predecessor, the OPTIMAX® Plus), which is geared for the Clinical Diagnostics lab, and the GENOMX 6000™, for Drug Discovery labs. Both systems BioGenex systems perform “All-in-One, All-at-Once” functions for cell and tissue testing, such as Immunohistochemistry (IHC) and In Situ Hybridization (ISH).

As an example, a determination module used in the system, computer-readable media and methods as disclosed herein for measuring biomarker level measures the level of at least one biomarker polypeptide. For instance, the determination module is configured to detect the total level (i.e. amount) of at least one biomarker protein of Tables 4 and 5 using any known systems for automated protein expression analysis, including for example, but not limited mass spectrometry systems including MALDI-TOF, or Matrix Assisted Laser Desorption Ionization-Time of Flight systems; SELDI-TOF-MS ProteinChip array profiling systems, e.g. Machines with Ciphergen PROTEIN BIOLOGY SYSTEM II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GENECHIP® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GENETITAN™ Instrument, GENECHIP® Array Station, HT Array; an automated ELISA system (e.g. DSX® or DS2® form Dynax, Chantilly, Va. or the ENEASYSTEM III®, TRITURUS®, The MAGO® Plus); Densitometers (e.g. X-Rite-508-SPECTRO DENSITOMETER®, The HYRYS™ 2 densitometer); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); and radio isotope analyzers (e.g. scintillation counters).

In some embodiments, the system, computer-readable media and methods as disclosed herein are used to measure at least one biomarker level in the biological sample obtained from an individual.

In some embodiments, the system, computer-readable media and methods as disclosed herein is used to measure at least one biomarker level in a sample which is obtained from a mammal, for example a human. In some embodiments, the individual has at least one symptom of as discussed herein.

In some embodiments, the system, computer-readable media and methods as disclosed herein is used to measure at least one biomarker level in a biological sample obtained from an individual who has experienced one or more symptoms of SSc, NSF or other chronic fibrotic disease.

In some embodiments, the system, computer-readable media and methods as disclosed herein comprises a determination module which has been configured to measure the level of an additional agent in the biological sample, for example, albumin band integrin.

In some embodiments, the system, computer-readable media and methods as disclosed herein is used to measure at least one biomarker level in a sample to indicate if an individual has, or is at risk of SSc, NSF or other chronic fibrotic disease. Accordingly, in some embodiments, the system, computer-readable media and methods as disclosed herein is used to identify if an individual has SSc, NSF or other chronic fibrotic disease.

Another aspect of the present invention relates to a method of treating an individual identified to have SSc, NSF or other chronic fibrotic condition comprising; (a) measuring if the individual has, or is likely to have or is at risk of having SSc, NSF or other chronic fibrotic condition by measuring at least one biomarker level in a sample obtained from the individual, and if high levels (e.g. at least about 2-standard deviations above a reference level for the measured biomarker) of the biomarker protein exists in the sample from the individual, it indicates that the individual is likely to have SSc, NSF or other chronic fibrotic condition, and (b) administering an appropriate treatment to the individual determined to likely have SSc, NSF or other chronic fibrotic condition, where an appropriate treatment can be determined by an ordinary physician, for example by a tissue biopsy.

In one embodiment, the method is performed on an individual who has experienced or exhibited symptoms of SSc, NSF or other chronic fibrotic disease.

In one embodiment, the diagnostic tool or device is used to test a biological sample from an individual who has experienced or exhibited symptoms of SSc, NSF or other chronic fibrotic disease.

The device or methods as disclosed herein can be used to assess samples from a individual at one or more indicated times following specific experienced symptoms of the individual, throughout the treatment regime and also during the remission period in order to monitor for relapse, such as at about 1 week, 2-5 months, 10 months, 1 year, 2 years, 3 years, 4 years etc.

The present invention can be defined in any of the following alphabetized paragraphs:
[A] A method for determining the likelihood of an individual having an active fibrotic condition comprising the steps of: (a) measuring an amount of at least one biomarker protein selected from the group consisting of ENO1, RCN-3 and PEDF, wherein the at least biomarker protein is measured alone or in combination with biomarker proteins RCN-1 and/or (α-SMA, wherein the biomarker proteins are found a biological sample obtained from the individual; (b) comparing the amounts of step (a) with a reference amount, wherein the amount of step (a) is greater than the reference amount indicates that the individual has an increased likelihood of having an active fibrotic condition.
[B] The method of paragraph 1, wherein the active fibrotic condition is selected from the group consisting of scleroderma, nephrogenic systemic fibrosis, fasciitis, scleromyxedema, retroperitoneal fibrosis, pulmonary fibrosis, liver fibrosis, chronic graft versus host disease and chronic allograft rejection.
[C] The method of paragraph [A] or [B], wherein a combination of the amount of biomarker proteins to be determined is selected from the group consisting of ENO1 only, RCN-3 only, PEDF only, and all other combinatorial permutations of the five biomarker proteins; wherein the combination is not RCN-1 only and is not α-SMA only.
[D] The method of any of paragraphs [A]-[C], wherein the biological sample is from an extracellular source.
[E] The method of paragraph [D], wherein the extracellular source is selected from the group consisting of whole blood, plasma, serum, serum, bronco alveolar lavage, cerebrospinal fluid, and spent culture media of an ex vivo culture of a tissue excised from the individual.
[F] The method of any of paragraphs [A]-[C], wherein the biological sample is from an intracellular source.
[G] The method of paragraph [F], wherein the intracellular source is a tissue excised from the individual.
[H] The method of any of paragraphs [A]-[G], wherein the measurement is performed by mass spectrometry, an antibody-based analytical method or RT-qPCR.
[I] The method of any of paragraphs [A]-[H], wherein the amount of step (a) is two standard deviations greater than the average reference amount.
[J] The method of any of paragraphs [A]-[I], wherein the reference amount is an average amount from a population of healthy individuals who have not been diagnosed with an active fibrotic condition.
[K] A method of prognosis evaluation in an individual diagnosed with and being treated for an active fibrotic condition, the method comprising: (a) at a first time point, measuring an amount of at least one biomarker protein selected from the group consisting of ENO1, RCN-3, α-SMA, RCN-1 and PEDF, wherein the determination is not RCN-1 only and is not α-SMA only, wherein the biomarker protein are from a biological sample obtained from the individual; (b) at a second time point, measuring an amount of at least one biomarker protein selected from the group consisting of ENO1, RCN-3, α-SMA, RCN-1 and PEDF, wherein the determination is not RCN-1 only and is not α-SMA only, wherein the second time point is after the first time point, wherein the biomarker protein are from a biological sample obtained from the individual and wherein a combination of biomarker proteins selected for measurement is the same at both first and second time points; (c) comparing the amount of step (a) with the amount of step (b), wherein the amount of step (b) is less than the amount of step (a) indicates that the treatment is effective in the individual having an active fibrotic condition.
[L] The method of paragraph [K], wherein the amount of step (b) is at least two standard deviations less than the amount of step (a).
[M] The method of paragraph [K] or [L], wherein the active fibrotic condition is selected from the group consisting of scleroderma, nephrogenic systemic fibrosis, fasciitis, scleromyxedema, retroperitoneal fibrosis, pulmonary fibrosis, liver fibrosis, chronic graft versus host disease and chronic allograft rejection.
[N] The method of any of paragraphs [K]-[M], wherein a combination of the amount of biomarker proteins to be measured is selected from the group consisting of ENO1 only, RCN-3 only, PEDF only, and all other combinatorial permutations of the five biomarker proteins, wherein the combination is not RCN-1 only and not α-SMA only.
[O] The method of any of paragraphs [K]-[N], wherein the biological sample is from an extracellular source.
[P] The method of paragraph [0], wherein the extracellular source is selected from the group consisting of whole blood, plasma, serum, serum, bronco alveolar lavage and spent culture media of ex vivo culture of a tissue excised from the individual.
[Q] The method of any of paragraphs [K]-[N], wherein the biological sample is from an intracellular source.
[R] The method of paragraph [Q], wherein the intracellular source is a tissue excised from the individual.
[S] The method of any of paragraphs [K]-[R], wherein the measurement is performed by mass spectrometry, an antibody-based analytical method or RT-qPCR.
[T] A device comprising: (a) at least one protein-binding agent which specifically binds to at least one biomarker protein selected from the group of: ENO1, RCN-3, and PEDF and at least one protein-binding agent which specifically binds to RCN-1 and/or α-SMA; (b) at least one solid support for the at least one protein-binding agent in step (a), wherein the protein-binding agent is deposited on the solid support.
[U] The device of paragraph [T], wherein the at least one protein-binding agent deposited on the solid support specifically binds to the biomarker protein of ENO1 of SEQ ID NO: 3.
[V] The device of paragraph [T], wherein the at least one protein-binding agent deposited on the solid support specifically binds to the biomarker polypeptide of RCN-3 of SEQ ID NO: 4.
[W] The device of paragraph [T], wherein the at least one protein-binding agent deposited on the solid support specifically binds to the biomarker polypeptide of PEDF of SEQ ID NO: 2.
[X] The device of any of paragraphs [T]-[W], wherein the solid support is in the format of a dipstick, a microfluidic chip or a cartridge.
[Y] The device of any of paragraphs [T]-[X], wherein the protein-binding agent is an antibody, an antibody-binding moiety, antibody fragment, aptamer, small molecule or variant thereof.
[Z] The device of any of the paragraphs [T]-[Y], wherein the protein-binding agent deposited on the device specifically binds to the biomarker protein when the level of the biomarker protein is at least two standard deviations above a reference level for that biomarker protein.
[AA] A kit comprising: (a) a device according to any of paragraphs [T]-[Z]; and (b) a first agent, wherein the first agent produces a detectable signal in the presence of a protein-binding agent which deposited on the device is specifically bound to a biomarker protein selected from the group of: ENO1, RCN-3, α-SMA, RCN-1 and PEDF.
[BB] The kit of paragraph [AA], further comprising a second agent, wherein the second agent produces a different detectable signal in the presence of a second protein-binding agent deposited on the device which is specifically bound to a second biomarker protein selected from the group of: ENO1, RCN-3, α-SMA, RCN-1 and PEDF, wherein the first and second biomarker are not the same.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

This invention is further illustrated by the following example which should not be construed as limiting. The contents of all references cited throughout this application, as well as the figures and table are incorporated herein by reference.

### EXAMPLES

### Example 1

### Proteomic Analysis of Systemic Fibrosis (SSC) and Nephrogenic Systemic Fibrosis (NSF) Secretome

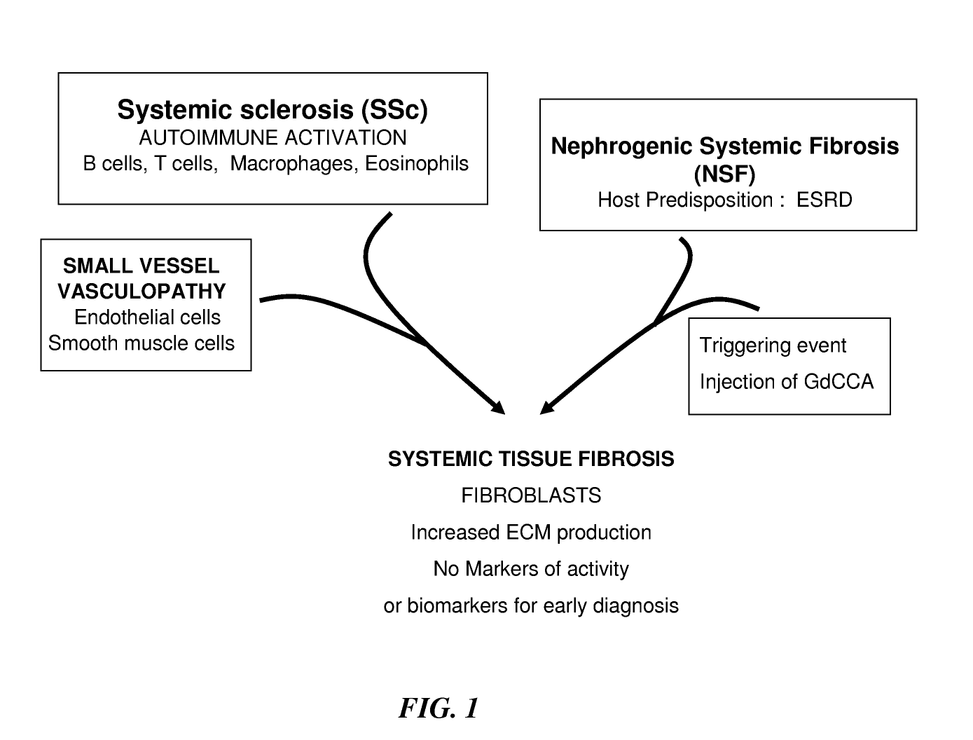

The most frequent systemic fibrotic disorder is SSc, a disease characterized by excessive deposition of collagen and other connective tissue macromolecules in skin and multiple internal organs, prominent and often severe alterations in the microvasculature, and humoral and cellular immunologic abnormalities (FIG. 1). The most apparent and almost universal clinical features of SSc are related to the severe fibrotic changes occurring in multiple tissues and very prominently in the microvasculature (Varga and Abraham, J., Clin. Invest., 2007, 117:557-567; Jimenez and Derk, Ann. Intern. Med., 2004, 140:37-50). The extent and rate of progression of tissue fibrosis is of paramount importance in measuring the clinical features and the prognosis of SSc. Indeed, fibrosis of the skin correlates with both survival and functional limitations (Denton C P, et. al., Nat. Clin. Pract. Rheumatol., 2006, 2:134-144; Mayes M D., Rheum. Dis. Clin. North. Am., 2003, 29:239-254).

Although there has been substantial interest in the identification of biomarkers that allow early diagnosis and assessment of disease activity or that carry a predictive prognostic in SSc (Doran J P and Veale D J, Rheumatology (Oxford) 2008, 47:v36-38; Hummers L K, Rheumatology (Oxford), 2008, 47:v21-22; Castro S V and Jimenez S A, Biomarkers Med., 2010, 4:133-147) including global gene expression and microarray studies (Sargent J L, et al., Curr. Rheumatol. Rep. 2008, 10:205-211; Farina G., et al., Arthritis Rheum., 2010, 62:580-588), fully validated biomarkers reflecting the fibrotic process are not available. The clinical semi-quantitative assessment of skin thickness by palpation (modified Rodnan skin score; mRSS) is considered the gold standard and the only primary outcome measure used in clinical trials of SSc disease modifying agents. This subjective and highly variable assessment is fraught with inaccuracies as pointed out recently (Clements P J, et al., J. Rheumatol., 1993, 20:1892-1896). It is, therefore, generally accepted that the development of objective and reliable markers reflecting the severity of tissue fibrosis would be of great value for improving the performance of clinical trials and the accurate assessment of the efficacy of a given treatment. Furthermore, such markers would allow a reduction in the number of patients needed for clinical trials to achieve statistical power and would offer an objective and quantitative method independent of the subjective assessment of the investigators involved in the study.

NSF is a recently recognized fibrotic disorder occurring in patients with renal insufficiency following exposure to Gd-containing contrast agents employed for magnetic resonance imaging (Mendoza F A., et. al., Semin Arthritis Rheum., 2006, 35:238-49; Cowper S E, et. al., Am. J. Dermatopathol., 2001, 23:383-393; Jimenez S A., et. al., Arthritis Rheum., 2004, 50:2660-2666; Grobner and Prischl, Kidney Int., 2007, 72:260-264). Affected tissues from NSF patients display a remarkable fibrotic process and like SSc fibroblasts (see FIG. 1), fibroblasts cultured from patients with NSF produce increased levels of collagens and other extracellular matrix proteins which are maintained in vitro for several passages (Edward M, et. al., Br J. Dermatol., 2007, 156:473479).

Here, the inventors describe the results of a proteomic analysis of the secretome of fibroblasts from SSc and NSF patients; the results allowed the identification of specific biomarker proteins that are useful to assess the extent and severity of the fibrotic process, and for prognostic evaluation of treatment.

### Method

### Cell Culture

Dermal fibroblasts were isolated from punch or excissional biopsies of affected forearms obtained from patients with SSc fulfilling the ACR criteria for disease classification (Subcommittee for scleroderma criteria of the American Rheumatism Association: Preliminary criteria for the classification of systemic sclerosis (Scleroderma). Arthritis Rheum., 1980, 23:581-590) and from patients with a typical clinical presentation of NSF associated with recent exposure to Gd-containing magnetic resonance imaging contrast agents described previously (Jimenez S A, et al., Arthritis Rheum., 2004, 50:2660-2666; Mendoza F A, et al., Semin Arthritis Rheum 2006, 35:238-249). All SSc patients had the diffuse cutaneous subset as defined by LeRoy et al., (J. Rheumatol. 1988, 15:202-205) and in all patients the disease was of recent onset (<18 months), and rapidly progressive. The SSc and NSF patients from whom the biopsies were obtained were matched for age and gender and had not received corticosteroids, anti-fibrotic or immunosuppressive therapy. Punch biopsies from age/sex matched normal subjects were used as controls. The biopsies, processed within 1 h of excision, were split in two halves; one half was formalin-fixed and paraffin-embedded for histopathology and immunohistochemistry, and the other half processed for establishment of dermal fibroblast cell strains. For this purpose, the skin biopsies were minced with a scalpel and small pieces of tissue were placed on plastic culture dishes, and then covered with tissue culture media which was changed every 3-5 days until visible outgrowth of cells was obtained in approximately 2-3 weeks. The cells were then enzymatically disassociated with 1 mg/ml trypsin at 37° C. for 5-30 min and then subcultured exactly as described by LeRoy et al., (LeRoy E C., J. Exp. Med., 1972, 135:1351-1362). These culture conditions allowed the expansion of pure fibroblast populations without any contamination with epithelial or endothelial cells or cells of hematopoietic origin. Dermal fibroblasts from patients with SSc or NSF were subcultured and used between passage 5 and 6 to avoid the loss of the fibroblast overproducer phenotype which is preserved for at least 12 serial passages under these in vitro culture conditions (LeRoy E C., supra; Castro S V and Jimenez S A, Biomarkers Med., 2010, 4:133-147). Normal fibroblasts were also matched for passage number. Cell strains were established from three different patients with SSc, three different patients with NSF, and three normal individuals. All cultures were grown to confluence in DMEM (Invitrogen, Carlsbad, Calif.) supplemented with vitamins (Cellgro, Manassas, Va.) 10% fetal bovine serum (FBS; Invitrogen), and antibiotics (Cellgro). Once the cultures reached confluency, they were washed twice and incubated for 18 h in serum free DMEM.

### Preparation of Supernatants for 2D Gel Electrophoresis.

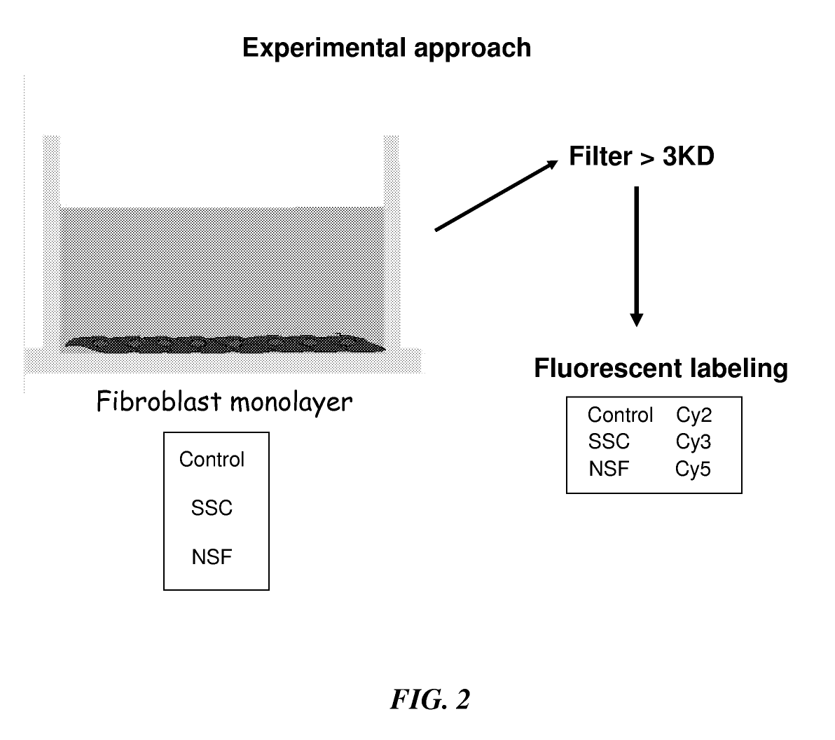

The general experimental approach is shown schematically in FIG. 2. Supernatants from the three SSc, three normal or three NSF cultures were pooled separately and each pool concentrated 40 times by 18 h centrifugation on Centrisep 3000 kD columns at 4° C., according to the manufacturer's instructions. To avoid any differences in the starting sample, the same volume of tissue culture media (20 ml) obtained when the cells reached over-confluency for at least 24 h was used in the final step. Protein concentration in the culture supernatants was determined using the GE Healthcare Quant Kit (Piscataway, N.J., USA). The samples were then brought to pH 8-8.5 with 1 M NaOH to optimize fluorescent tagging. For each gel, 50 μg of protein for each category was added to 400 pM of Cy2, Cy3 or Cy5 fluorescent tags, and allowed to incubate on ice for 30 min. The labeling reaction was quenched by addition of 1 μ1 of 10 mM lysine and subsequent incubation on ice for 15 min. For 2D electrophoresis, the three samples (normal, SSc and NSF) were pooled, brought up to 350 μ1 in 8M urea, 4% CHAPS, and supplemented with DTT (final concentration 13 mM) and IPG Buffer 3-10 (final concentration 2%) prior to 2-DE as described previously in Sharma K, et al., Proteomics 2005, 5:2648-2655 and Ramachandra Rao S P, et al., Am. J. Physiol. Renal. Physiol., 2007, 292:F1182-1189. Two dimensional-difference (DIGE) labeling and two dimensional (2D) gel electrophoresis.

For each gel, 18 cm, pH 3-10 immobilized pH gradient (IPG) strips were rehydrated at 30 V for 12 h in 350 μ1 of sample, using an IPGphor (GE Healthcare). Once rehydration was complete, samples were focused at 500 V for 1 h, 1000 V for 1 h, and finally 8000 V for 6 h. Immediately after completion. IPG strips were processed for separation by SDS-PAGE. In order to reduce the disulfide bonds in the focused proteins in preparation for the second dimension, IPG strips were incubated for 15 min in equilibration buffer I consisting of 0.375 M Tris-HCl pH 8.8, 6 M urea, 2% SDS, 20% glycerol, and 13 mM DTT. The IPG strips were soaked in equilibration buffer II for an additional 15 mM to alkylate the sulfitydryl groups. Buffer II is identical to buffer I with the exception that 2.5% (w/v) iodoacetamide is used instead of DTT. The strips were embedded in 0.7% w/v agarose on top of 12.5% acrylamide slab gels. Second dimension separations were performed on a DALT6 platform (GE Healthcare). IPG strips were electrophoresed at 2 W/gel for 30 min, followed by 20 W/gel until the dye front reached the bottom of the gel. The gel was rinsed in deionized water and scanned using the DIGE-enabled Typhoon Scanner (GE Healthcare). Following scanning of the images, DeCyder 5.01 software (GE Healthcare) was used for differential gel analysis. The 2-D images from the different samples were then compared using the DIA module of DeCyder using a value of 1000 as the initial estimate of protein spots present. DIA analysis allows for the direct comparison of intensities of specific protein spots between different samples within the same gel (Karp N A, et al., Proteomics 2004, 4:1421-1432).

### Tryptic Digestion of Isolated Protein Spots.

The 2D gel was post stained with SYPRO Ruby and rescanned using the Typhoon Scanner. The resulting gel images were matched back to the master DIGE image and spots of interest designated for picking from the SYPRO image. Protein spots of interest were prepared for mass spectral analysis by the Spot Handling Workstation (GE Healthcare). SYPRO-stained spots of interest were automatically cut from the gel and washed twice with 50 mM ammonium bicarbonate in 50% methanol. The plugs were then dehydrated in 75% acetonitrile for 10 min and dried under a stream of air. Trypsin (10 μl of 20 μg/ml in 20 mM ammonium bicarbonate) was added to each plug and incubated for 2 h at 37° C. Following digestion the resulting tryptic peptides were extracted twice into 50% acetonitrile, 0.1% formic acid, and then dried completely under a stream of air.

### Mass Spectrometry (MS) Protein Identification

Proteins were identified via liquid chromatography-mass spectrometry (LC/MS) using a Thermo Scientific ProteomeX Workstation consisting of a Surveyor HPLC front end, followed by an LCQ DecaXP Plus ion-trap mass spectrometer. The dried down peptide extracts were resuspended in 15 μl 1% formic acid, and 10 μl were loaded onto a Thermo Hypersil-Keystone BioBasic C18 column (0.18×100 mm). The peptides were separated at a flow rate of 4 μl/min using a linear gradient of 2-50% acetonitrile in 0.1% formic acid over 45 min. As the peptides were eluted from the column, they were subjected to a full MS scan, followed by tandem mass spectrometry of the three largest peaks. The resulting ms/ms spectra were analyzed using SEQUEST. Protein identifications were considered valid if they met the HUPO protein identification filter (X_{Corr}≧1.9, 2.2. or 3.75 for z=1, 2, and 3 respectively, deltaCN≧0.1; and Rsp≦4) as described previously in the Human Plasma Proteome Project (Omenn G S, et. al., Proteomics, 2005, 5:3226-3245).

### Confocal Microscopy Validation

To validate the results obtained by the proteomic analyses, reticulocalbin-1 (RCN-1) one of the proteins which displayed the highest differential expression, was selected for semi-quantitative assessment of its abundance in normal, SSc, and NSF skin. RCN-1 was analyzed by immunofluorescence using a RCN-1 rabbit polyclonal antibody (Bethyl Laboratories, Montgomery, Tex.). Isotype control staining was performed using Rabbit IgG (Sigma, St Louis. MO). Secondary antibodies were affinity purified sheep (FAb)₂ anti rabbit IgG-Cy3 conjugated (Sigma). Paraffin embedded sections from skin were deparaffinized with two changes of xylene for 10 min each and then 2 changes of ethanol for 5 min each. Antigen retrieval was performed by boiling the tissue sections in 10 mM citrate buffer pH 6.0 for 20 min. The sections were then rinsed in PBS for 2 min and incubated with 5% normal sheep serum for 20 min at room temperature to block non-specific binding sites. The primary antibody incubation step was performed overnight at 4° C. followed by incubation with the sheep polyclonal antibody (1:200). The unbound antibodies were removed from the sections with three changes of PBS for 2 min each after each incubation. Tissue sections were counterstained with DAPI and analyzed using a Zeiss LSM 510 META Confocal Laser Scanning Microscope System. Zeiss META confocal software was used in balancing signal strength. The breakthrough of the DAPI signal into the Red and the Green channel was recorded separately and subtracted from the DAPI blue channel. The Zeiss META enhancement software was used in balancing the signal strength and the image was scanned 8-fold to separate signal from noise. Panels were assembled using. Photoshop software without any RGB modification. RCN-3 was analyzed by the same method using anti-RCN-3 antibody.

### Western Blots

Sera from ten SSc patients and two healthy normal volunteers were assayed for RCN-1. All sera were from patients who fulfilled the ACR classification criteria for SSc described previously herein and have diffuse subset of the disease classified according to LeRoy et al., (J. Rheumatol 1988, 15:202-205). Sera were diluted 1:10 with saline solution and electrophoresed on a 10% Tris/glycine SDS gel. Primary antibody against human RCN-1 (Bethyl Laboratories) was used at a 1:1000 dilution, incubated overnight at 4° C. Anti rabbit HRP conjugated antibodies at 1:5000 dilution were used as secondary antibody.

### Results

### Two Dimensional-Difference Gel Electrophoresis (2D-D1GE)

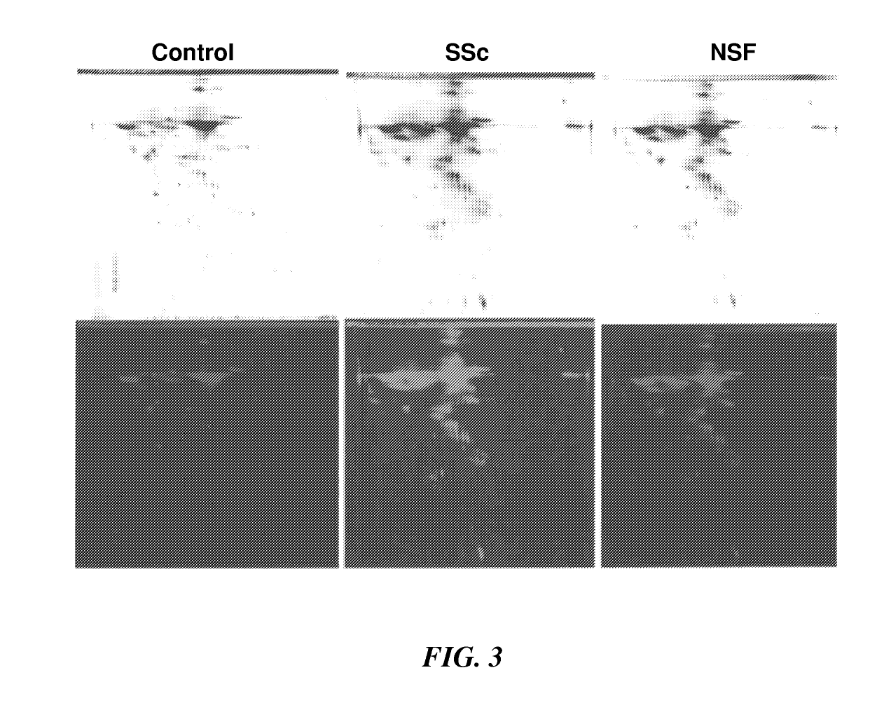

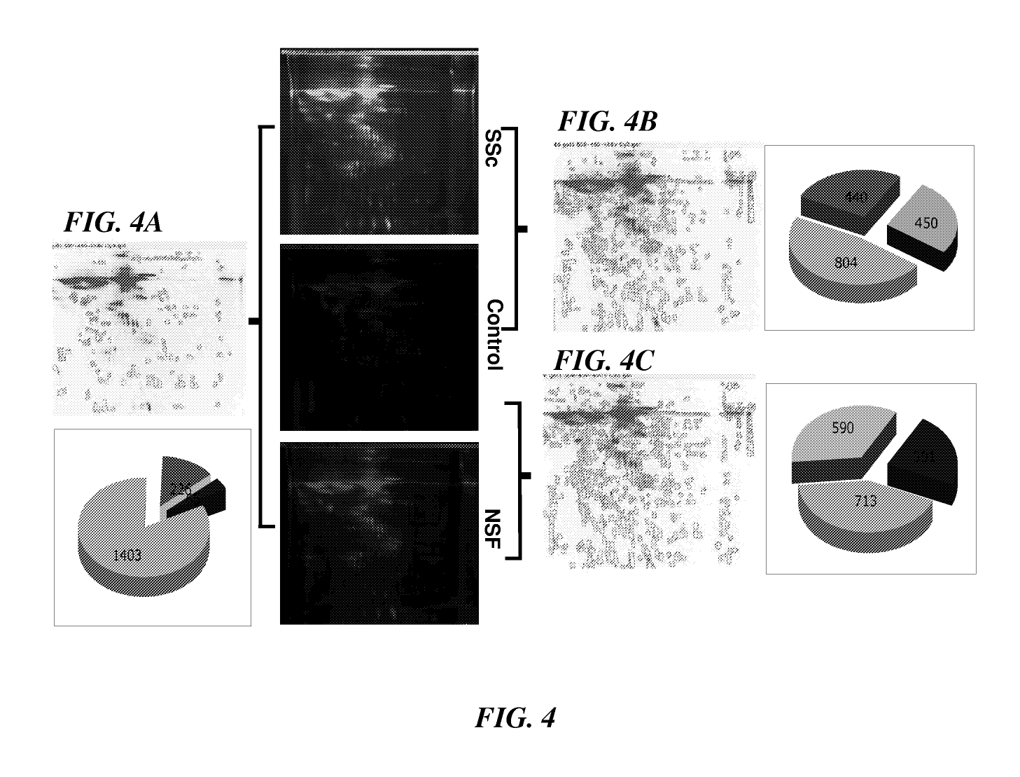

2D-DIGE was used to analyze the supernatants from three SSc, three NSF and three normal fibroblast cultures. The secreted proteins from normal fibroblast cell strains were pooled and conjugated with Cy2 fluorochrome; the secreted proteins from SSc fibroblast cell strains were pooled and conjugated with Cy3; and the secreted protein from the NSF fibroblast cell strains were pooled and conjugated with Cy5. The images detected for each fluorochrome are shown in FIGS. 3 and 4. A total of 1694 spots were detected on the 2D electrophoresis. DIGE software analysis allowed quantitative determination of each fluorescent signal in every detected spot (world wide website download of DIGE software described supra). The volume ratio analysis comparison of the secretome of normal fibroblasts with that of SSc fibroblasts indicated that 450 spots were increased (>twofold) in the SSc fibroblast secretome, whereas 440 were decreased (<50%) and 804 had similar expression (FIG. 4B). The differential analysis of NSF versus normal fibroblast secretome identified 391 spots increased in NSF, 590 decreased and 713 with similar expression (FIG. 4C). In contrast, the differential analysis of SSc versus NSF fibroblast secretomes revealed that 1403 spots (85%) had similar expression whereas 65 spots were increased in the SSc sample and 226 spots were increased in the NSF sample (FIG. 4A).

### Volume Ratio Overlay

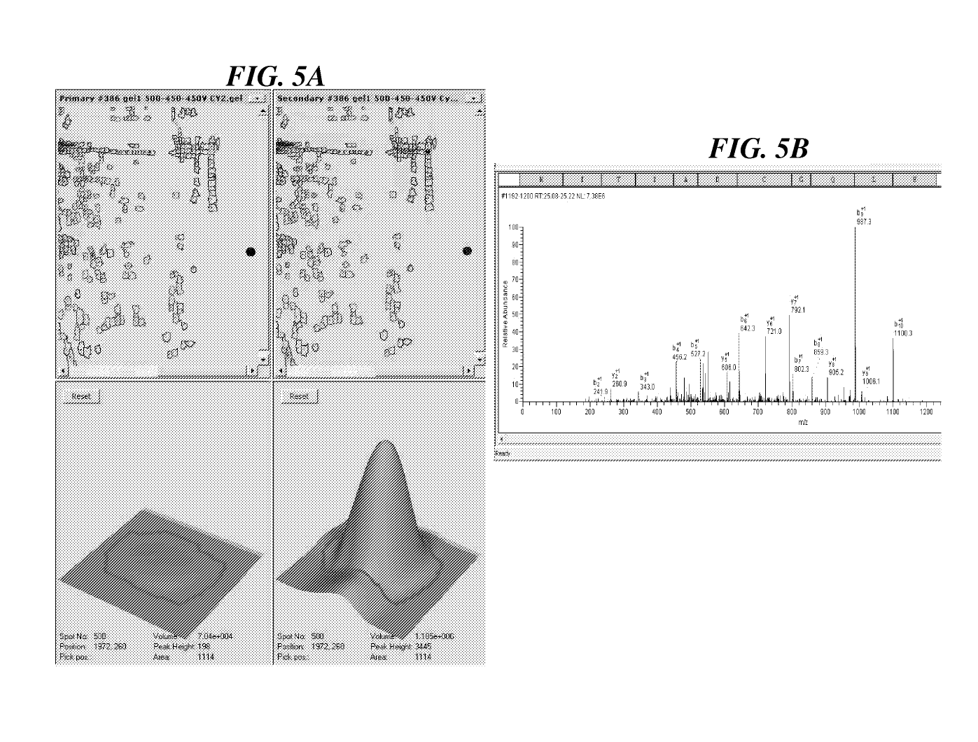

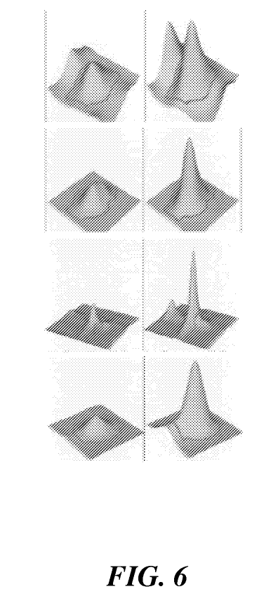

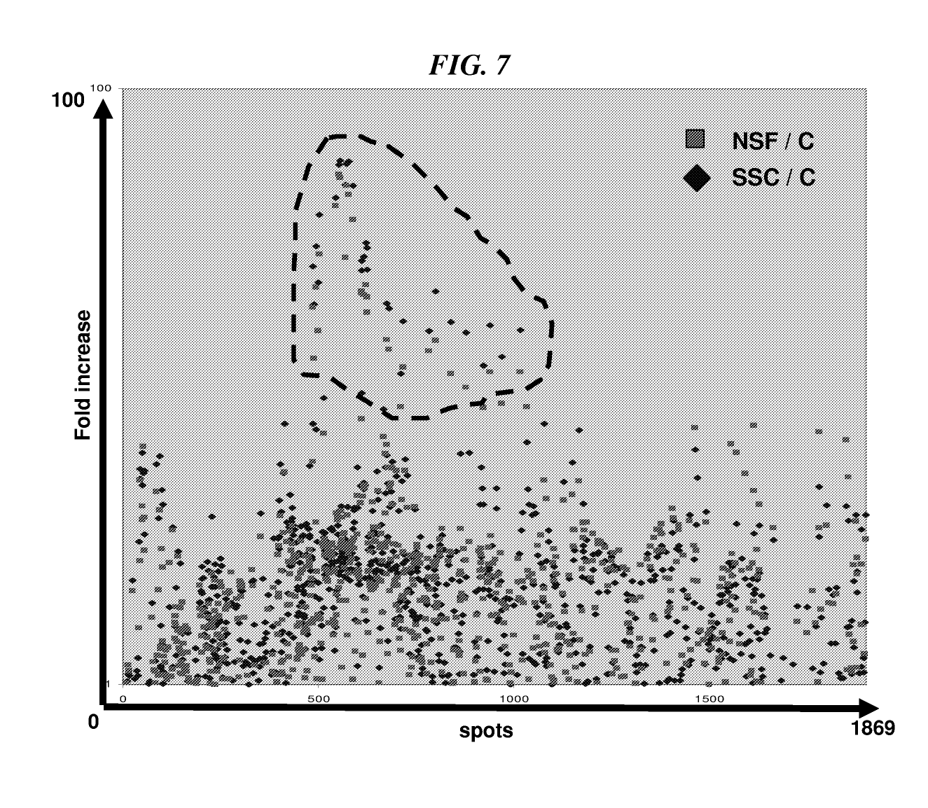

Each protein spot fluorescence intensity was calculated by DIGE software and the ratio between SSc and normal and that of NSF and normal were calculated (FIG. 5A). To analyze the differentially expressed proteins with common quantitative changes among the NSF and SSc fibroblast secretomes, each spot was assigned a progressive number, and the fluorescence intensity ratio between SSc fibroblast secretome and normal fibroblast secretome was plotted against the spot number. The same analysis was performed for the volume ratio between the NSF and normal fibroblast secretomes and the two series of dot-plots were overlaid in the same graph. FIG. 7 shows the comprehensive chart of all the biomarker protein spots obtained in the mass 2D DIGE analysis showing the fold increase. For the purpose of biomarker identification, mass spectrometry analysis of the spots that were most upregulated in both SSc and NSF samples were selected. Each of the spots with the highest level of increased compared to the normal secretome was digested and analyzed by mass spectrometry. A three dimensional view displaying the intensity of fluorescence as volume of some of the spots selected for analysis is shown in FIG. 6.

### Mass Spectrometry Identification of the Shared Changes in SSC and NSF Fibroblast Secretomes

The list of the upregulated proteins identified by mass spectrometry is shown in Table 4 and the complete list of all differentially expressed proteins which were identified by mass spectrometry is shown it Table 5. Of note, the most upregulated protein, reticulocalbin-3 (RCN-3, accession number 28626510) and the fourth most upregulated protein, reticulocalbin-1 (RCN-1, accession number 4506455) both belong to the CREC (acronym for Cab45, reticulocalbin. ERC-45, and calumenin) family of calcium binding proteins residing in the endoplasmic reticulum and which are very likely involved in trafficking and transport of newly synthesized protein (Honore B and Vorum H., FEBS Lett., 2000, 466:11-18; Vorum H, et. al., Exp. Cell Res., 1999, 248:473-481; Fukuda T, et. al., J. Histochem. Cytochem., 2007, 55:335-345). Remarkably, calumenin (accession number 4502551), identified to be upregulated in average 2.5 fold, also belongs to the same family of proteins. Of further interest was the fact that the second and third most upregulated proteins identified were SPARC or osteonectin (OCN, accession number 4507171) and the α2 chain of type I collagen (COL1A2, accession number 48762934). Both proteins have been shown to be upregulated in SSc tissues by several independent, hypothesis driven studies (Verrecchia F., et. al., Rheumatology, 2007 (Oxford), 46:833-41; Davies Calif., et. al., Rheumatology (Oxford) 2006, 45:1349-1355; Macko R F., et. al., J. Rheumatol., 2002, 29:2565-2570).

RCN-1 Expression is Increased in vivo in SSC and NSF

To validate the proteomic results, the tissue expression levels of RCN-1, a protein found to be among the most upregulated in the secretomes of both SSc and NSF fibroblasts, was examined in affected SSc and NSF skin. For this purpose, the analyzes were by way of immunofluorescence followed by confocal microscopy of the tissue of expression of RCN-1 in three skin biopsies from SSc patients, three skin biopsies from an affected area of skin of a patient with NSF, and three normal skin biopsies. FIG. 8A shows representative images demonstrating substantially increased levels of RCN-1 in the dermis of the SSc and the NSF skin samples analyzed. The distribution of RCN-1 was both cellular and pericellular, with a remarkable accumulation surrounding small vessels. Quantitative analysis of fluorescence performed in the three SSc, three NSF, and three normal skin samples showed that the overall levels of RCN-1 expression in SSc skin biopsies were 2.5-fold higher than that in normal skin, whereas the levels in the NSF sample was 3.1 fold higher than that in normal skin (FIG. 8B). To further validate the results obtained by proteomic approach and confocal microscopy analysis, Western blots were performed to examine the presence of RCN-1 in the sera from SSc patients. FIG. 8C shows that a band of protein recognized by the specific anti-RCN-1 antibody and of the appropriate molecular size was clearly detectable in sera from the ten SSc patients examined as well as in the sera from the two healthy volunteers. The volunteers' sera had significantly less level of RCN-1 in the Western blot.

### Conclusion

Although activated fibroblasts play the central role in the development of tissue and organ fibrosis in SSc and other chronic fibrotic diseases, there is currently a lack of reliable fibroblast activity markers (Castro S V and Jimenez S A, Biomarkers Med., 2010, 4:133-147). To identify possible markers that directly reflect the level of fibroblast activity in various fibrotic diseases and which may be detectable in the sera of afflicted individuals, the differences in the secretome of normal versus activated or profibrotic fibroblasts were analyzed by employing a proteomic approach. Owing to the complexity and heterogeneity of skinpathology in SSc, it was considered likely the possibility that some of the genes/proteins detected in the SSc fibroblast secretome can reflect other concomitant pathophysiological events such as immune activation or vasculopathy. Therefore, as the target of the studies was the identification of markers that specifically reflected tissue fibrosis and fibroblast activation, the differential analysis the secretome of cultured fibroblasts derived from affected skin of NSF was performed in parallel. NSF is another fibrotic condition in which vasculopathy and autoimmunity are not considered to be present.

The rationale for studying fibroblast culture supernatants for biomarker identification relies on the assumption that a soluble secreted or shed cellular product released and present in the tissue culture media could be detected in vivo in serum and other bodily fluids, and, therefore, be easily accessible for assay by venipuncture. The approach has been extremely informative for a variety of cancer associated molecules (Kulasingam V and Diamandis E P, Int J Cancer 2008, 123:2007-2012; Sardana G, et al., Clin. Chem. 2007, 53:429-437). Another important advantage of the proteomic approach is that any biomarker identified employing proteomics is completely unbiased and only depends upon the sensitivity of the differential analysis of two dimensional gel electrophoresis with the accuracy of mass spectrometry identification. The utilization of proteomic approaches for biomarker identification and for the study of disease pathogenesis for numerous diseases including SSc (Rottoli P, et al., Proteomics, 2005, 5:1423-1430; Bogatkevich G S, et al., Am. J. Physiol. Lung Cell Mol. Physiol., 2008, 295:L603-611; Aden N, et al., Rheumatology (Oxford) 2008, 47:1754-1760) has been rapidly growing and is highly promising.

The comparative analysis of the differentially expressed proteins from SSc and NSF fibroblast secretomes revealed a pattern of shared alterations in these two clinically and etiologically different fibrotic conditions. Of interest, the mass spectrometry identification of the most increased spots revealed proteins that are already known to be part of the profibrotic phenotype of SSc fibroblasts such as COL1A2, α-SMA and osteonectin. The identification of these proteins by the comprehensive proteomic approach described here is in agreement with the numerous studies that identified their increase as a typical feature of the profibrotic phenotype of SSc fibroblasts and, most importantly, is a very strong internal validation of the functional relevance of the procedure employed herein to discovery biomarkers of fibrotic conditions.

Of remarkable interest was the identification of several proteins that were not previously known to be associated with the profibrotic phenotype. Three of the 10 most upregulated proteins identified belonged to the CREC family of proteins, which are calcium binding proteins residing in the endoplasmic reticulum that appear to participate in traffick and transport of newly synthesized proteins (Honore B, et al., FEBS Lett, 2000, 466:11-18; Vorum H, et al., Exp Cell Res 1999, 248:473-481; Fukuda T, et al., J Histochem Cytochem 2007, 55:335-345). Furthermore, given the co-localization of the CREC proteins with protein disulfide isomerases in the endoplasmic reticulum and their opposite regulation during endoplasmic stress responses (Mintz M, et al., J Proteome Res., 2008, 7:2435-2444) they may also be involved in the regulation of the proper folding of procollagen molecules and play a role in the complex pathway of procollagen assembly processing and secretion mediated by protein disulfide isomerase (Bassuk J A, et al., Matrix 1989, 9:244-258; Kellokumpu S, et al., J. Biol. Chem., 1997. 272:2770-2777; Wilson R, et al., J. Biol. Chem., 1998, 273:9637-9643; Ko M K and Key E P, Exp. Cell Res., 2004, 295:25-35). The presence of intracellular proteins in the concentrated supernatants may reflect cellular death, as well as increased cell turnover or the increased secretion of cell microparticles as has been recently suggested (Guiducci S, et al., Arthritis Rheum., 2008, 58:2845-2853; Jüngel A, et al., Arthritis Rheum., 2007, 56:3564-3574).

An in vivo validation of the proteomic results was performed to support the contention that these identified proteins can be bone fide potential biomarkers of the fibrotic process. RCN-1 was selected since it is the most abundant member of the family of reticulocalbins. Investigation was performed to determine whether RCN-1 was actually overexpressed in vivo in SSc and NSF-affected skin and whether it was detectable in the sera of affected individuals. Therefore, by way of immunofluorescence studies of skin samples from three SSc patients and three NSF patients that compared the amount of expression of RCN-1 with the levels detectable in normal skin biopsies, it was observed that RCN-1 epitopes were detectable in skin and expression in the dermis. Moreover, RCN-1 was significantly higher in both SSc and NSF skin samples than in normal skin samples. To determine whether RCN-1 was detectable in the serum, Western blots sera from ten SSc patients and two normal subjects were analyzed. The results indicated that a protein band was recognized by specific RCN-1 antibodies and the band was of a correct molecular size. The results also indicated that RCN-1 was indeed present and detectable in sera from these patients and normal subjects.

### Example 2

### Diagnostic Lateral Flow Immunoassay (LFIA) Test Strips-Design 1

The levels of biomarker proteins associated with SSc, NSF or other chronic fibrotic diseases described herein can be determined using lateral flow immunoassay (LFIA) test strips as illustrated in FIG. 9-10. This test strip can be used in point-of-care testing (POCT). The test strip has a sample (S) position at one end of the test strip and a control (C) position found at the opposite end the test strip (FIG. 9A). There is a test (T) position located at the middle of the test strip, between S and T. For this embodiment of a test strip, the solid support 91 can be made of plastic or other non porous material, supporting the matrix 93. Located at S is a defined quantity of dehydrated anti-biomarker protein antibody. The defined quantity of dehydrated anti-biomarker protein antibody, when rehydrated, will bind at saturation a fixed amount of biomarker antigen, meaning that this fixed amount of biomarker protein will completely occupy all of the Fv binding sites of that defined quantity of antibody. If there is additional biomarker protein in excess of the fixed amount of biomarker that is required to bind all the antibodies deposited at position S, the excess biomarker proteins will be free and are not bound to any antibody in the form of an antibody-biomarker complex. The fixed amount of biomarker protein is the predetermined reference level of biomarker protein which is the level found in healthy individuals who do not have SSc, NSF or other chronic fibrotic conditions. The predetermined reference level can be about the amount of two standard deviations above the average amount found in a population of healthy individuals. In other embodiments, the predetermined reference level can be about three, four, five, six, seven, eight, nine or ten standard deviations above the average amount. The antibody at position S can be conjugated to colloidal gold beads or colored latex beads for visualization purposes. At position T, there is a defined quantity of biomarker protein immobilized on the test strip. This is the same biomarker protein that binds the antibody deposited at position S. At position C, there is another immobilized protein, an antibody immunoreactive to the anti-biomarker protein antibody located at the S position (FIG. 9).

The following is a description on how to use and interpret the results obtained for the test strip shown in FIG. 9. A sample of serum is applied at S. The water in the serum rehydrates the dehydrated anti-biomarker protein antibody that has been deposited at S. The dehydrated anti-biomarker protein antibody can be labeled with colloidal gold beads or colored latex beads. The biomarker protein in the serum binds to this rehydrated anti-biomarker protein antibody to form an antibody-biomarker complex. Any biomarker protein in the serum that is in excess of the rehydrated anti-biomarker protein antibody deposited at S will be free and is not bound to any antibody. A mixture of antibody-biomarker complex and free antibody or free biomarker will move by capillary action away from position S and will move toward the T position and subsequently to the C position. When the biomarker protein of interest is below the reference level, the mixture of antibody and biomarker protein will contain free anti-biomarker protein antibody and antibody-biomarker protein complexes. At position T, any free anti-biomarker protein antibody will bind to the immobilized biomarker protein at T. The localized concentration of free anti-biomarker protein antibody that is colloidal gold or latex bead labeled will become visible as a colored line at the T position (FIG. 10B). There is free antibody only when the biomarker protein in the serum is below the threshold reference value found in healthy humans, which is the predetermined reference level of biomarker protein. When the protein of interest is at or above the predetermined reference level, the mixture of antibody and biomarker protein will contain all antibody-biomarker protein complexes and no free anti-biomarker protein antibody. At the T position, there will be no anti-biomarker protein antibody captured by the immobilized biomarker protein. Thus there will be no colloidal gold or latex bead labeled anti-protein antibody accumulation, and the area remains clear (FIG. 10A). At position C, the antibody-biomarker complex formed initially at S will be bound and captured by the immobilized antibody immunoreactive against the anti-protein antibody coming from the S position. This will in turn result in a concentration of a colloidal gold or latex bead labeled anti-protein antibody accumulated at the C position and will become visible as colored line at the C position. The C position result serves as a test control to indicate that there is functional anti-protein antibody in the test material and should always be present (FIGS. 10A and 10B). When sufficient amount of labeled anti-biomarker protein antibody from the complex accumulates at C, a band becomes visible here. A band at C indicates that labeled antibody from the S position had moved to the C position. Therefore, a band at C indicates that the band at T is not a false positive. Arrowheads indicate the boundary limit that a serum sample should not cross on the test strip.

FIG. 10A-10D show the possible outcomes and interpretations of the results for such a test strip. FIG. 10A shows no band at position T but a distinct band at position C, indicating that the biomarker protein level is above the pre-determined reference level. This result means that SSc, NSF or other chronic fibrotic condition is indicated. FIG. 10B shows a band at position T and a distinct band at position C, indicating that the biomarker protein level is below the pre-determined reference level and SSc, NSF or other chronic fibrotic condition is not indicated. FIG. 10C shows a band at position T but no band at position C, indicating that the data at T may be a false positive. FIG. 10D shows no band at either positions T and C, indicating the data at T may be a false negative. Both FIGS. 10C and 10D indicate invalid data and the lateral flow immunoassay should be repeated with a new test strip.

The defined quantity of dehydrated anti-protein antibody at the S position is such that there is just enough antibody to bind the biomarker protein from the sample (e.g. serum) when the biomarker protein is at the reference/control level. The reference/control level can be the level of the biomarker found in the samples of healthy individuals. Therefore, when the biomarker protein is at or above the reference level, all of the anti-biomarker antibody at the S position will be bound to the biomarker protein in the form of biomarker protein-antibody complex; there will be no free anti-biomarker protein antibody present.

The choice of the anti-biomarker protein antibody placed at the S position can be any antibody that is specifically immunoreactive to any of the protein of interest, e.g. biomarkers described herein. The antibody can be monoclonal, polyclonal, or a mixture of both monoclonal and polyclonal antibodies. Antibody-based moiety can also be used.

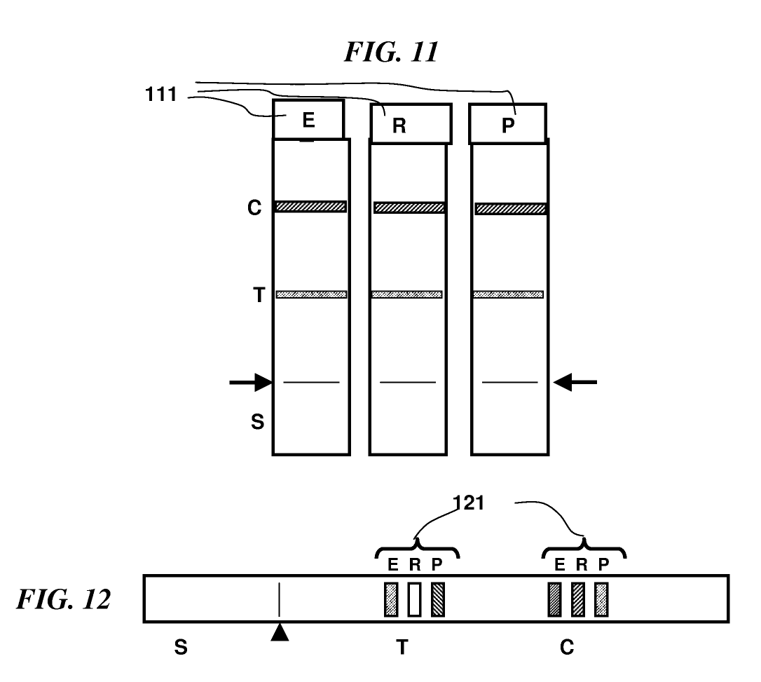

When only one biomarker protein is studied, the S position should have only one anti-biomarker protein antibody that specifically immunoreactive with just that one biomarker of interest (FIG. 11). A kit comprising test strips for use as POCT can have several single biomarker protein test strips. The kit can test for only one biomarker or more than one biomarker proteins. In this embodiment, the test strip can be labeled 111 on one end to identify the biomarker protein the test strip is used for, e.g. the label “E” represents α-enolase 1 (ENO1); “R” represents reticulocalbin 3 (RCN-3); and “P” represents pigment epithelium-derived factor (PEDF) (see FIG. 11). On the other hand, if more than one, e.g. three biomarker proteins are to be studied simultaneously, the S position can have three different types of anti-biomarker protein antibodies, each type specifically immunoreactive to one biomarker protein and does not exhibit cross-reactivity with the other two non-ligand proteins (FIG. 12). Arrowheads indicate the boundary limit that sample should not cross on the membrane. At positions T or C, up to three bands can be visible, each band corresponding to each of the biomarker protein that is being tested. When three proteins are to be studied simultaneously, all three protein types can be represented at the T position and at their respective quantities (FIG. 12). FIG. 12 shows an alternative design where three proteins can be studied simultaneously on the same test strip. The positions of the expected results in the T and C positions for each biomarker are indicated 121.

The test strip can be designed in a form of a dipstick test strip as described in FIG. 9B. As a dipstick test strip, the strip is dipped into a sample (e.g. serum) at the S position end with sample level not to exceed the boundary limit. The strip is then laid horizontally with the membrane surface facing up on a flat surface. A fixed amount of time is given for the antibody re-hydration, capillary action, and antibody biomarker protein binding reaction to take place. At the end of the fixed time, there should be visible bands at the C position and depending on the level of the protein of interest, there may or may not be a visible band at the T position (FIG. 10). FIG. 11 shows a method of using three separate dipstick test strips to test for the three biomarkers of interest. Each dipstick test strip is labeled 111 to indicate which biomarker protein is being tested. A diagnostic kit can comprise multiple types of single biomarker test strips, a type for each biomarker of interest.

### Example 3

### Diagnostic Lateral Flow Immunoassay (LFIA) Test Strips-Design 2

An alternative embodiment of the lateral flow immunoassay (LFIA) test strips for measuring the level of biomarker protein level is illustrated in FIG. 13A-D. This test strip can be used in point-of-care testing (POCT). Here the test strip contains two different anti-biomarker protein antibodies specific for the same biomarker; each antibody binds the biomarker at a different epitope. This is a double sandwich LFIA test strip. The first antibody is labeled (e.g. colored latex beads), deposited on the solid support matrix but is not immobilized on it, (i.e. the antibody is mobile), and is deposited in excess at the S position. The second anti-biomarker protein antibody is not labeled but is immobilized and is in excess at position T. This second anti-biomarker protein antibody binds an epitope on the biomarker that is not affected by the binding of the first antibody. At position C, there is an excess of non-labeled antibody against the anti-biomarker antibody deposited at the S position. The antibody at C serves to capture any free labeled anti-biomarker antibody migrating from S. When sufficient free labeled anti-biomarker antibody is accumulated at C, a visible band appears. The band is a control to confirm that the band(s) observed on the test strip at T are due to the mobile antibody at the S position.

Initially before use, there should be no visible band at position T and C of the test strip (FIG. 13B). When a fluid sample (e.g. serum) is place at the S position, the water in the serum rehydrates the dehydrated anti-biomarker protein antibody that has been deposited at S. The dehydrated anti-biomarker protein antibody can be labeled with colloidal gold beads or colored latex beads. The biomarker protein in the serum binds to this rehydrated anti-biomarker protein antibody to form an antibody-biomarker complex. A mixture of free anti-biomarker antibody and biomarker protein:antibody complexes is formed. The mixture migrates by capillary action towards the T and the C positions. The second anti-biomarker antibody immobilized at T will capture all the biomarker protein: antibody complexes but not the free anti-biomarker protein antibody. The localized concentration of anti-biomarker protein:antibody complexes that is colloidal gold or latex bead labeled will become visible as a colored line at the T position (FIG. 13C). Only when the biomarker protein is at or above the reference level will sufficient labeled antibody be captured at T to produce a visible band (FIG. 13C). When the biomarker is below the reference level, no visible band should appear at the T position (FIG. 13D).

At position C, free anti-biomarker antibody initially from S will be bound and captured by the immobilized antibody immunoreactive against the antibody coming from the S position. This will in turn result in a concentration of a colloidal gold or latex bead labeled anti-protein antibody accumulated at the C position and will become visible as colored line at the C position. The C position result serves as a test control to indicate that there is functional anti-protein antibody in the test material and should always be present. A band at C indicates that labeled antibody from S had moved to C. Therefore, a band at C indicates that the band at T is not a false positive or that the absence of a band at T is a false negative.

### Example 4

### Diagnostic Lateral Flow Immunoassay (LFIA) Test Strips-Design 3

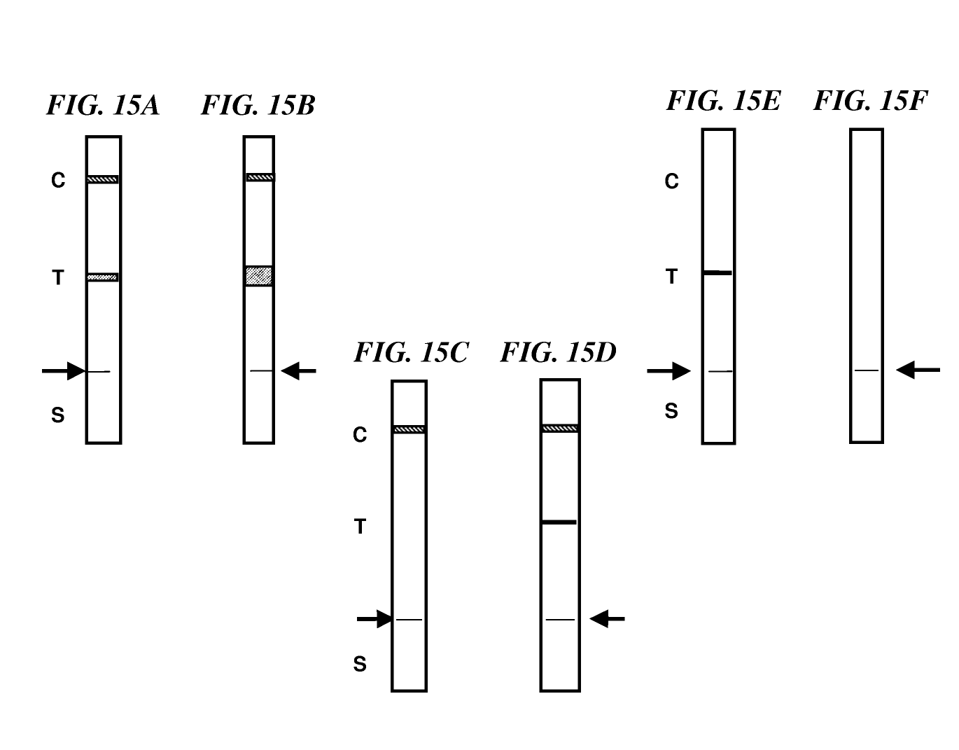

An alternative embodiment of the lateral flow immunoassay (LFIA) test strips for measuring the level of biomarker protein level is illustrated in FIG. 14. This test strip can be used in point-of-care testing (POCT). The test strip is as described in FIG. 9 having a sample (S), a test (T), and a control (C) positions, all three spatially arranged as shown in FIG. 9 and FIG. 13. For this embodiment of a test strip, the solid support 141 can be made of plastic or other non porous material, supporting the matrix 143. In this embodiment, the S position contain an excess amount of dehydrate anti-biomarker protein antibody (first antibody) that can be labeled (e.g. colloidal gold or color latex bead). Similar to the embodiments in FIG. 9-13, the anti-biomarker protein antibody at S is mobile; once the antibody is re-hydrated, the antibody moves by capillary action towards the T and C positions.

The T position contains a second anti-biomarker protein antibody that is also immunoreactive to the biomarker protein of interest, but to a different epitope on the biomarker (FIG. 14). This second antibody is in excess and is immobilized on the matrix. This second anti-biomarker protein antibody binds a part of the biomarker protein that is different from the part of the protein that is bound by the first anti-biomarker protein antibody found at the S position. In this embodiment, the second antibody at the T position will bind and capture both free unbound biomarker protein and biomarker protein-antibody complexes, and concentrate them at the T position.

The C position contains a defined quantity of biomarker protein immobilized on the membrane (FIG. 14B). The defined quantity is the predetermined reference value of the biomarker protein being analyzed on the test strip. The reference/control level can be the level of the biomarker found in the samples of healthy individuals. When the excess free anti-biomarker protein antibody from the S position arrives and bind the immobilized biomarker protein at C, gradually accumulation at C produces a concentration of labeled first antibody will become visible as a colored line at the C position (FIG. 15A-D).

An application of a fluid sample (e.g. serum) at the S position will re-hydrate the excess amount of anti-biomarker protein antibody there. All of the biomarker protein of interest should be bound to the excess anti-biomarker protein antibody deposited at S. A fluid mixture of free biomarker protein antibody and biomarker protein-antibody complex is formed and will move along the membrane by capillary action towards the T position and then subsequently to the C position. At the T position, all of the biomarker protein-antibody complex will be captured and immobilized by the second anti-biomarker protein antibody. The localized concentration of biomarker protein-antibody complexes, wherein the anti-biomarker protein antibody that is colloidal gold or latex bead labeled, will become visible as a colored line at the T position (FIG. 15A, B, D). With increasing amount of biomarker protein-antibody complexes and concentration at the T position, the colored line expands and develops into a band. The greater the level of biomarker in the sample, the wider the colored band at the T position (FIGS. 15A and B).

When excess free (unbound) anti-biomarker protein antibody from the S position arrives to the C position and bind to the immobilized reference amount of biomarker protein there, another color line become visible. Since there is a reference amount of immobilized biomarker protein at the C position, the thickness of the visible colored line at the C position defines the reference value of protein. By comparing the thickness of the color band at the T and C positions on the same test strip, one can estimate whether the biomarker protein level is below or greater than the reference value of the protein. When the biomarker protein level is equal or greater than the reference value, the color band at the T position will be equal or larger than the color band at the C position respectively (FIGS. 15A and B) and SSc, NSF or other chronic fibrotic disease is indicated. When the biomarker protein level is below the threshold level, the color band at the T position will be smaller or even absent than the color band at the C position (FIGS. 15C and D) and therefore SSc, NSF or other chronic fibrotic disease is not indicated. The C position band also serves as a test control to confirm that there is functional anti-biomarker antibody at the S position and that the functional anti-biomarker protein antibody is derived from the S position (FIGS. 15E and F). FIG. 15E shows a band at position T but no band at position C, indicating that the data at T may be a false positive. FIG. 15F shows no band at either positions T and C, indicating the data at T may be a false negative. Both FIGS. 15E and 15F indicate invalid data and that the lateral flow immunoassay should be repeated with a new test strip.

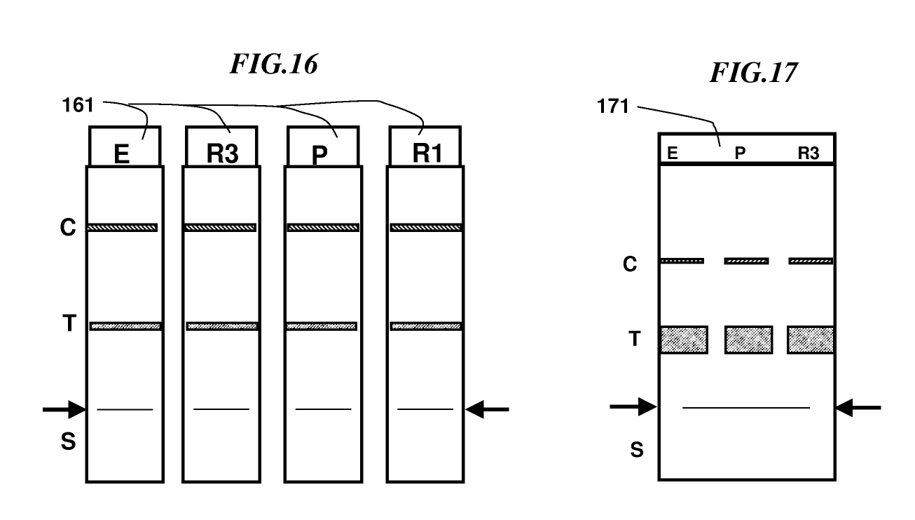

When only one biomarker protein is studied, the S position should have only one anti-biomarker protein antibody that specifically immunoreactive with just that one biomarker of interest. A kit comprising test strips for use as POCT can have several single biomarker protein test strips. The kit can test for only one biomarker or more then one biomarker proteins. In this embodiment, the test strip can be labeled 161 on one end to identify the biomarker protein the test strip is used for, e.g. the label “E” represents α-enolase 1 (ENO1); “R3” represents reticulocalbin 3 (RCN-3); “P” represents pigment epidelium-derived factor (PEDF) and “R1” represents reticulocalbin 1 (RCN-1) (see FIG. 16). FIG. 16 shows a method of using four separate dipstick test strips to test for the four biomarkers of interest. Such test strip can be the component of a diagnostic kit. Each dipstick test strip is labeled 161 to indicate which biomarker protein is being tested. A diagnostic kit can comprise multiple types of single biomarker test strips, a type for each biomarker of interest.

On the other hand, if more than one, e.g. three biomarker proteins are to be studied simultaneously, the S position can have three different types of anti-biomarker protein antibodies, each type specifically immunoreactive to one biomarker protein and does not exhibit cross-reactivity with the other two non-ligand proteins (FIG. 17). FIG. 17 shows an alternative embodiment of a test strip where three biomarker proteins can be studied simultaneously on the same test strip. The positions for each biomarker on the single strip are indicated 171.

The test strip can be designed in a form of a dipstick test strip as described in FIG. 14B). As a dipstick test strip, the strip is dipped into a sample (e.g. serum) at the S position end with sample level not to exceed the boundary limit. The strip is then laid horizontally with the membrane surface facing up on a flat surface. A fixed amount of time is given for the antibody re-hydration, capillary action, and antibody biomarker protein binding reaction to take place. At the end of the fixed time, there should be visible bands at the C position and depending on the levels of the biomarker protein(s) of interest, there may or may not be a visible band at the T position (FIG. 15) and the bands can be any different thickness.

### Example 4

### Diagnostic ELISA Assay Design 1

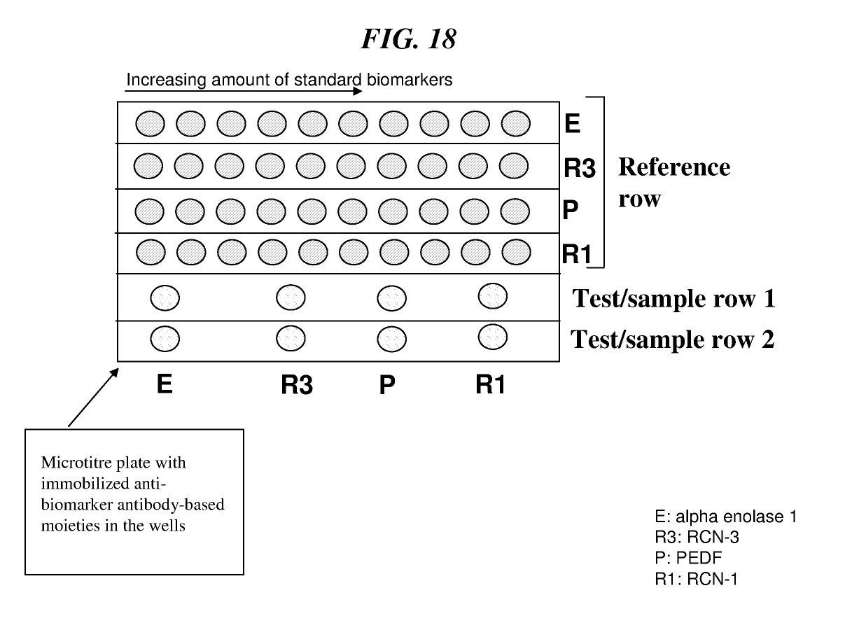

The levels of biomarker proteins described herein can be measured using an ELISA assay illustrated in FIG. 18. An ELISA assay comprises performing a standard titration assay and a sample assay in order to determine the amount of protein present in the sample. As shown in FIG. 18, the ELISA assay microtiter plate consists of four reference rows: a reference row for each of the four biomarker proteins, α-enolase 1 (ENO1); reticulocalbin 3 (RCN-3); pigment epithelium-derived factor (PEDF) and reticulocalbin 1 (RCN-1), and two test/sample rows. The reference rows and sample wells for the different protein of interest are labeled (FIG. 18). Excess amounts of anti-biomarker protein antibodies are immobilized in the wells of plate. There is a specific antibody for each of the biomarker protein of interest. Standard amounts of protein ranging from 0-50 pg/ml, ng/ml, μg/ml, or mg/ml are placed in the reference rows to create a standard curve for each of the biomarker protein of interest. The biological sample is placed in the sample wells. Subsequently, an enzyme-labeled (e.g. horse-radish peroxidase) anti-biomarker protein antibody specific for each of the biomarker protein of interest is added to the wells. The mixtures in the wells are allowed to incubate at room temperature for 90 min and the liquid is decanted. The wells are washed five times with deionized water. The, an aliquot of 3,3′,5,5′ tetramethylbenzidine (TMB) reagent is added into each well. The mixture is gently mixed for 10 seconds and incubated at room temperature (18-25° C.) for 20 minutes. The enzymatic reaction is terminated by adding 1N HCl. Gentle agitation is carried out till all the blue color changes to yellow color completely. The amount of color by-product is determined by reading its absorbance at 450 nm with a microtiter well reader. The A₄₅₀ correspond to the amount of biomarker protein in the well. The amount of the biomarker protein in a test sample can be estimated from the A₄₅₀ obtained from the sample wells and the standard curve obtained from the reference wells.

### Example 5

### Diagnostic ELISA Assay Design 2

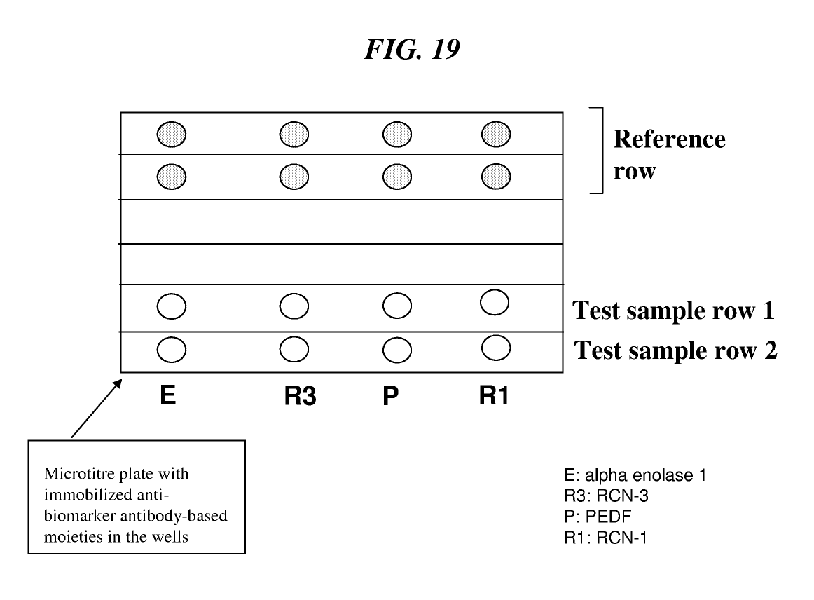

Alternatively, the second ELISA assay as shown in FIG. 19 can be used. The reference rows and sample wells for the different biomarker protein of interest are labeled (FIG. 19). Excess amounts of anti-biomarker antibodies are immobilized in the wells of plate, with a specific antibody for each of the protein of interest. A fixed amount of each of protein of interest is placed in duplicate reference wells. This fixed amount is the reference value corresponds to the average amount of the protein found in the corresponding biological samples of healthy individuals who do not have SSc, NSF or other fibrotic diseases. In some embodiments, the reference value can also be the average amount plus twice of the standard deviations obtained from the data of a population of healthy individuals who do not have SSc, NSF or other fibrotic diseases. The biological sample, e.g. serum, is also placed in the duplicate sample wells. The assay plate is process as described herein. The A₄₅₀ obtained from the sample wells are compared with those obtained for the corresponding reference rows in order to determine whether there is an increase or decrease in the amount of the protein in the sample.

TABLE 1
Scleroderma Disease Classification

| Localized Scleroderma | Systemic sclerosis (SSc)* |
|---|---|
| En coup de saber - linear | Limited - sclerosis limited to hands, distal |
| lesions of scalp or face | extremities, and CREST syndrome-a form of |
| Linear scleroderma - linear | limited systemic sclerosis with calcinosis, |
| lesions seen in childhood | Raynaud's phenomenon, esophageal |
| Limited/generalized | dysmotility, sclerodactyly, and |
| morphea - circumscribed | telangiectasias |
| patches of sclerosis | Diffuse - sclerosis of proximal extremities, |
| | trunk, and face |
| | Scleroderma sine scleroderma - organ |
| | fibrosis; no skin lesions |

*With varying degrees of internal organ involvement

TABLE 2
Major Clinical Manifestations of Systemic sclerosis

```
Cutaneous
    Skin fibrosis
    Swollen fingers and hands
    Flexion contractures and tendon friction rubs
Vascular
    Raynaud's phenomenon and nail fold capillary changes
    Digital ischemia/ulcers
Pulmonary
    Alveolitis and interstitial fibrosis
    Pulmonary hypertension
    Restrictive lung disease (decreased thoracic compliance)
    Aspiration pneumonitis (esophageal reflux and dysmotility)
Cardiac
    Arrhythmia and conduction disorders (myocardial fibrosis)
    Heart failure
Musculoskeletal/Rheumatologic
    Arthralgia and myalgias
    Erosive arthropathy (rare)
    Median nerve entrapment
Gastrointestinal
    Gastroesophageal reflux and peptic stricture
    Esophageal dysmotility
    Gastric bleeding from ectatic vessels
    (gastric antral vascular ectasia, or “watermelon stomach”)
    Malabsorption and diarrhea (bacterial overgrowth)
    Primary biliary cirrhosis
Endocrine
    Hypothyroidism
The Cleveland Clinic Foundation 2004
```

TABLE 3
ACR Diagnostic Criteria for Systemic sclerosis

| Major Criteria | Minor Criteria* |
|---|---|
| Proximal sclerodermatous skin | Sclerodactyly |
| changes (proximal to the | Digital pitting scars of fingertips |
| metacarpophalangeal joints) | or loss of substance of the |
| | distal finger pad |
| | Bibasilar pulmonary fibrosis |

*The patient should fulfill the major criterion or two of the three minor criteria Adapted from the Subcommittee for Scleroderma Criteria of the American Rheumatism Association Diagnostic and Therapeutic Criteria Committee. Preliminary criteria for the classification of systemic sclerosis (scleroderma). Arthritis Rheum., 1980, 23: 581-590.

TABLE 4
Proteins most highly differentially upregulated in both SSc and
NSF secretomes identified by mass spectrometry

| | Accession | Fold |
|---|---|---|
| Protein ID | Number | Change |
| Reticulocalbin-3 (SEQ. ID. NO: 4) | 28626510 | 26.18 |
| Osteonectin (SEQ. ID. NO: 7) | 4507171 | 18.28 |
| α2 chain of type I collagen | 48762934 | 17.69 |
| (SEQ. ID. NO: 8) | | |
| Reticulocalbin-1 (SEQ. ID. NO: 1) | 4506455 | 17.69 |
| Tropomyosin 4 (SEQ. ID. NO: 9 & 10) | 4507651 | 14.32 |
| Enolase 1 (SEQ. ID. NO: 3) | 450371 | 10.28 |
| Calreticulin precursor (SEQ. ID. NO: 11) | 4757900 | 7.08 |
| Actin, alpha 1 (SEQ. ID. NO: 6) | 4501881 | 5.56 |
| Pigment epithelium derived factor (SEQ. | 39725934 | 2.95 |
| ID. NO: 2) | | |

TABLE 5
List of all protein spots that had a positive identification by MS
with their relative ratio in the two conditions.

| C/S | C/NF | | | |
|---|---|---|---|---|
| Ratio | Ratio | Description | Mass | Accession # |
| 3.32 | 2.71 | alpha-feto protein precursor | 68634 | 4501989 |
| 3.01 | 2.71 | alpha-feto protein precursor | 68634 | 4501989 |
| 3.17 | 2.58 | lactotransferrin | 78323 | 4505043 |
| 3.20 | 3.38 | Serine Proteinase Inhibitor, | 46,694 | 21361198 |
| | | clade A | | |
| 3.82 | 4.10 | Serine Proteinase Inhibitor, | 46,694 | 21361198 |
| | | clade A | | |
| 4.64 | 4.29 | prolyl 4-hydroxylase, | 57,082 | 20070125 |
| | | beta subunit | | |
| −3.83 | −7.08 | Calreticulin precursor | 48,113 | 4757900 |
| −2.17 | −10.28 | enolase 1 | 47,140 | 4503571 |
| −10.19 | −26.18 | reticulocalbin 3 | 37,471 | 28626510 |
| −3.75 | −1.29 | calumenin precursor | 37,085 | 4502551 |
| −10.88 | −23.26 | reticulocalbin 3 | 37,471 | 28626510 |
| −4.90 | −1.32 | calumenin precursor | 37,085 | 4502551 |
| −4.46 | −1.50 | calumenin precursor | 37,085 | 4502551 |
| −2.51 | −5.56 | actin, alpha 1 | 42,025 | 4501881 |
| −4.55 | −2.95 | PEDF | 46,284 | 39725934 |
| −9.03 | −17.69 | reticulocalbin 1 precursor | 38,867 | 4506455 |
| −8.51 | −18.28 | Osteonectin | 34,611 | 4507171 |
| −9.03 | −17.69 | alpha 2 type I collagen | 129,236 | 48762934 |
| −7.94 | −14.32 | Tropomyosin 4 | 28,506 | 4507651 |
| −1.67 | 1.30 | alpha 1 type I collagen | 138,828 | 4502945 |

C/S ratio: Normal fibroblast secretome/SSc fibroblast secretome.
C/NF ratio: Normal fibroblast secretome/NSF fibroblast secretome.

What is claimed:

1. A method for determining the likelihood of an individual having an active fibrotic condition comprising the steps of:
(a) measuring an amount of at least one biomarker protein selected from the group consisting of α-enolase (ENO1), reticulocalbin 3 (RCN-3) and pigment epithelium-derived factor (PEDF), wherein the at least biomarker protein is measured alone or in combination with biomarker proteins reticulocalbin 1 (RCN-1) and/or alpha smooth muscle actin (α-SMA), wherein the biomarker proteins are found a biological sample obtained from the individual;
(b) comparing the amounts of step (a) with a reference amount, wherein the amount of step (a) is greater than the reference amount indicates that the individual has an increased likelihood of having an active fibrotic condition.

2. The method of claim 1, wherein the active fibrotic condition is selected from the group consisting of scleroderma, nephrogenic systemic fibrosis, fasciitis, scleromyxedema, retroperitoneal fibrosis, pulmonary fibrosis, liver fibrosis, chronic graft versus host disease and chronic allograft rejection.

3. The method of claim 1, wherein a combination of the amount of biomarker proteins to be determined is selected from the group consisting of ENO1 only, RCN-3 only, PEDF only, and all other combinatorial permutations of the five biomarker proteins; wherein the combination is not RCN-1 only and is not α-SMA only.

4. The method of claim 1, wherein the biological sample is from an extracellular source or an intracellular source.

5. The method of claim 4, wherein the extracellular source is selected from the group consisting of whole blood, plasma, serum, serum, bronco alveolar lavage, cerebrospinal fluid, and spent culture media of an ex vivo culture of a tissue excised from the individual.

6. The method of claim 1, wherein the measurement is performed by mass spectrometry, an antibody-based analytical method or RT-qPCR.

7. The method of claim 1, wherein the amount of step (a) is two standard deviations greater than the average reference amount.

8. The method of claim 1, wherein the reference amount is an average amount from a population of healthy individuals who have not been diagnosed with an active fibrotic condition.

9. A method of prognosis evaluation in an individual diagnosed with and being treated for an active fibrotic condition, the method comprising:
(a) at a first time point, measuring an amount of at least one biomarker protein selected from the group consisting of α-enolase (ENO1), reticulocalbin 3 (RCN-3), alpha smooth muscle actin (α-SMA), reticulocalbin 1 (RCN-1) and pigment epithelium-derived factor (PEDF), wherein the determination is not RCN-1 only and is not α-SMA only, wherein the biomarker protein are from a biological sample obtained from the individual;
(b) at a second time point, measuring an amount of at least one biomarker protein selected from the group consisting of α-enolase (ENO1), reticulocalbin 3 (RCN-3), alpha smooth muscle actin (α-SMA), reticulocalbin 1 (RCN-1) and pigment epithelium-derived factor (PEDF), wherein the determination is not RCN-1 only and is not α-SMA only, wherein the second time point is after the first time point, wherein the biomarker protein are from a biological sample obtained from the individual and wherein a combination of biomarker proteins selected for measurement is the same at both first and second time points;
(c) comparing the amount of step (a) with the amount of step (b), wherein the amount of step (b) is less than the amount of step (a) indicates that the treatment is effective in the individual having an active fibrotic condition.

10. The method of claim 9, wherein the amount of step (b) is at least two standard deviations less than the amount of step (a).

11. The method of claim 9, wherein a combination of the amount of biomarker proteins to be measured is selected from the group consisting of ENO1 only, RCN-3 only, PEDF only, and all other combinatorial permutations of the five biomarker proteins, wherein the combination is not RCN-1 only and not α-SMA only.

12. A device comprising:
(a) at least one protein-binding agent which specifically binds to at least one biomarker protein selected from the group of: α-enolase (ENO1), reticulocalbin 3 (RCN-3), and pigment epithelium-derived factor (PEDF) and at least one protein-binding agent which specifically binds to reticulocalbin 1 (RCN-1) and/or alpha smooth muscle actin (α-SMA);
(b) at least one solid support for the at least one protein-binding agent in step (a), wherein the protein-binding agent is deposited on the solid support.

13. The device of claim 12, wherein the at least one protein-binding agent deposited on the solid support specifically binds to the biomarker protein of ENO1 of SEQ ID NO: 3.

14. The device of claim 12, wherein the at least one protein-binding agent deposited on the solid support specifically binds to the biomarker polypeptide of RCN-3 of SEQ ID NO: 4.

15. The device of claim 12, wherein the at least one protein-binding agent deposited on the solid support specifically binds to the biomarker polypeptide of PEDF of SEQ ID NO: 2.

16. The device of claim 12, wherein the solid support is in the format of a dipstick, a microfluidic chip or a cartridge.

17. The device of claim 12, wherein the protein-binding agent is an antibody, an antibody-binding moiety, antibody fragment, aptamer, small molecule or variant thereof.

18. The device of claim 12, wherein the protein-binding agent deposited on the device specifically binds to the biomarker protein when the level of the biomarker protein is at least two standard deviations above a reference level for that biomarker protein.

19. A kit comprising:
(a) a device according to claim 12; and
(b) a first agent, wherein the first agent produces a detectable signal in the presence of a protein-binding agent which deposited on the device is specifically bound to a biomarker protein selected from the group of: α-enolase (ENO1), reticulocalbin 3 (RCN-3), alpha smooth muscle actin (α-SMA), reticulocalbin 1 (RCN-1) and pigment epithelium-derived factor (PEDF).

20. The kit of claim 19, further comprising a second agent, wherein the second agent produces a different detectable signal in the presence of a second protein-binding agent deposited on the device which is specifically bound to a second biomarker protein selected from the group of: α-enolase (ENO1), reticulocalbin 3 (RCN-3), alpha smooth muscle actin (α-SMA), reticulocalbin 1 (RCN-1) and pigment epithelium-derived factor (PEDF), wherein the first and second biomarker are not the same.


SEQUENCE LISTING (27 sequences)

SEQ ID NO: 1 - protein, 331 aa, Homo sapiens
MARGGRGRRLGLALGLLLALVLAPRVLRAKPTVRKERVVRPDSELGERPPEDNQSFQYDHEAFLGKEDSKTFDQLTPDESKERLGKIVDRIDNDGDGFVTTEELKTWIKRVQKRYIFDNVAKVWKDYDRDKDDKISWEEYKQATYGYYLGNPAEFHDSSDHHTFKKMLPRDERRFKAADLNGDLTATREEFTAFLHPEEFEHMKEIVVLETLEDIDKNGDGFVDQDEYIADMFSHEENGPEPDWVLSEREQFNEFRDLNKDGKLDKDEIRHWILPQDYDHAQAEARHLVYESDKNKDEKLTKEEILENWNMFVGSQATNYGEDLTKNHDEL

SEQ ID NO: 2 - protein, 418 aa, Homo sapiens
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 3 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 4 - protein, 328 aa, Homo sapiens
MMWRPSVLLLLLLLRHGAQGKPSPDAGPHGQGRVHQAAPLSDAPHDDAHGNFQYDHEAFLGREVAKEFDQLTPEESQARLGRIVDRMDRAGDGDGWVSLAELRAWIAHTQQRHIRDSVSAAWDTYDTDRDGRVGWEELRNATYGHYAPGEEFHDVEDAETYKKMLARDERRFRVADQDGDSMATREELTAFLHPEEFPHMRDIVIAETLEDLDRNKDGYVQVEEYIADLYSAEPGEEEPAWVQTERQQFRDFRDLNKDGHLDGSEVGHWVLPPAQDQPLVEANHLLHESDTDKDGRLSKAEILGNWNMFVGSQATNYGEDLTRHHDEL

SEQ ID NO: 5 - protein, 377 aa, Homo sapiens
MCEEEDSTALVCDNGSGLCKAGFAGDDAPRAVFPSIVGRPRHQGVMVGMGQKDSYVGDEAQSKRGILTLKYPIEHGIITNWDDMEKIWHHSFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 6 - protein, 377 aa, Homo sapiens
MCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 7 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 8 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 9 - protein, 284 aa, Homo sapiens
MEAIKKKMQMLKLDKENAIDRAEQAEADKKAAEDKCKQVEEELTHLQKKLKGTEDELDKYSEDLKDAQEKLELTEKKASDAEGDVAALNRRIQLVEEELDRAQERLATALQKLEEAEKAADESERGMKVIENRAMKDEEKMEIQEMQLKEAKHIAEEADRKYEEVARKLVILEGELERAEERAEVSELKCGDLEEELKNVTNNLKSLEAASEKYSEKEDKYEEEIKLLSDKLKEAETRAEFAERTVAKLEKTIDDLEEKLAQAKEENVGLHQTLDQTLNELNCI

SEQ ID NO: 10 - protein, 248 aa, Homo sapiens
MAGLNSLEAVKRKIQALQQQADEAEDRAQGLQRELDGERERREKAEGDVAALNRRIQLVEEELDRAQERLATALQKLEEAEKAADESERGMKVIENRAMKDEEKMEIQEMQLKEAKHIAEEADRKYEEVARKLVILEGELERAEERAEVSELKCGDLEEELKNVTNNLKSLEAASEKYSEKEDKYEEEIKLLSDKLKEAETRAEFAERTVAKLEKTIDDLEEKLAQAKEENVGLHQTLDQTLNELNCI

SEQ ID NO: 11 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 12 - DNA, 21 bp, Artificial Sequence
  Description of Artificial Sequence Synthetic primer
tgcagacctcaatggtgacct

SEQ ID NO: 13 - DNA, 20 bp, Artificial Sequence
  Description of Artificial Sequence Synthetic primer
aaggcagtgaactcctcccg

SEQ ID NO: 14 - DNA, 20 bp, Artificial Sequence
  Description of Artificial Sequence Synthetic primer
gagctccgggacaatgataa

SEQ ID NO: 15 - DNA, 20 bp, Artificial Sequence
  Description of Artificial Sequence Synthetic primer
tgttccatccatctcgatca

SEQ ID NO: 16 - DNA, 20 bp, Artificial Sequence
  Description of Artificial Sequence Synthetic primer
tgtgcaggcttagagggact

SEQ ID NO: 17 - DNA, 20 bp, Artificial Sequence
  Description of Artificial Sequence Synthetic primer
gttcacggggactttgaaga

SEQ ID NO: 18 - DNA, 20 bp, Artificial Sequence
  Description of Artificial Sequence Synthetic primer
gacttccgggatctgaacaa

SEQ ID NO: 19 - DNA, 20 bp, Artificial Sequence
  Description of Artificial Sequence Synthetic primer
ccatagttggtggcctgact

SEQ ID NO: 20 - DNA, 20 bp, Artificial Sequence
  Description of Artificial Sequence Synthetic primer
ttcaatgtcccagccatgta

SEQ ID NO: 21 - DNA, 20 bp, Artificial Sequence
  Description of Artificial Sequence Synthetic primer
gaaggaatagccacgctcag

SEQ ID NO: 22 - DNA, 20 bp, Artificial Sequence
  Description of Artificial Sequence Synthetic probe
tgtgcaggcttagagggact

SEQ ID NO: 23 - DNA, 20 bp, Artificial Sequence
  Description of Artificial Sequence Synthetic probe
ggagttcactgcctttctgc

SEQ ID NO: 24 - DNA, 20 bp, Artificial Sequence
  Description of Artificial Sequence Synthetic probe
agcggaaatcctgggtaatt

SEQ ID NO: 25 - DNA, 20 bp, Artificial Sequence
  Description of Artificial Sequence Synthetic probe
acccacaatgtccccatcta

SEQ ID NO: 26 - DNA, 20 bp, Artificial Sequence
  Description of Artificial Sequence Synthetic probe
gaagggtgtctcaaaggctg

SEQ ID NO: 27 - protein, 6 aa, Artificial Sequence
  Description of Artificial Sequence Synthetic 6xHis tag
HHHHHH